(19) United States
(12) Patent Application Publication
(10) Pub. No.: US 2025/0325835 A1
(43) Pub. Date: Oct. 23, 2025

(54) SYSTEM, METHOD AND DEVICE FOR DELIVERY OF A THERAPEUTIC OR DIAGNOSTIC AGENT

(71) Applicant: Bayer Healthcare LLC, Whippany, NJ (US)

(72) Inventors: John Volkar, Valencia, PA (US); Joana Oliveira Rego Brilhante, Jersey City, NJ (US); Anne Berit Eid, Oslo (NO); Ellia Hu, Eastwood (AU); Hamsini Sundararaman, Monroeville, PA (US); Ashley Cockerham, Tulsa, OK (US); Winston Ge, Cambridge, MA (US); Andrew Jones, Roslindale, MA (US); Rich Miller, Needham, MA (US); Joe Ting, Acton, MA (US); Brian Yoo, Arlington, MA (US); Ryan Bayne, Ithaca, NY (US)

(21) Appl. No.: 19/252,857

(22) Filed: Jun. 27, 2025

Related U.S. Application Data
(63) Continuation of application No. 18/838,935, filed on Aug. 15, 2024.
(60) Provisional application No. 63/312,145, filed on Feb. 21, 2022.
(60) Provisional application No. 63/312,148, filed on Feb. 21, 2022.
(60) Provisional application No. 63/312,151, filed on Feb. 21, 2022.
(60) Provisional application No. 63/312,152, filed on Feb. 21, 2022.

(51) Int. Cl. A61N 5/10; A61M 5/00; A61M 5/14; A61M 5/165; A61M 5/168; A61M 5/172

(52) U.S. Cl. CPC A61N 5/1007; A61M 5/007; A61M 5/1409; A61M 5/165; A61M 5/16877; A61M 5/172; A61M 2005/1403; A61M 2205/15; A61M 2205/3379; A61N 2005/1021; A61N 2005/1022; A61N 2005/1095

(57) ABSTRACT

A fluid injector system includes at least one fluid reservoir having a first fluid reservoir configured for injecting a radiopharmaceutical and a radiation filter in fluid communication with the at least one fluid reservoir. The radiation filter is configured for retaining radioactive particles from the radiopharmaceutical passing though the radiation filter. The system further includes at least one sensor configured to detect radioactivity in at least one of the first fluid reservoir, the radiation filter, and a fluid path element in fluid communication with the radiation filter; and a controller in operative communication with the at least one sensor. The controller is programmed or configured to receive a radioactivity measurement from the at least one sensor and determine, based on the radioactivity measurement, that an amount of radioactive particles in at least one of the first fluid reservoir, the radiation filter, and the fluid path set satisfies a predetermined threshold.

### CROSS-REFERENCE TO RELATED APPLICATIONS

The present application is continuation of U.S. application Ser. No. 18/838,935, filed Aug. 15, 2024, which is a U.S. national phase application under 35 U.S.C. § 371 of PCT International Application No. PCT/US2023/062889, filed Feb. 20, 2023, and claims priority to U.S. Provisional Application No. 63/312,145, filed on Feb. 21, 2022; U.S. Provisional Application No. 63/312,151, filed on Feb. 21, 2022; U.S. Provisional Application No. 63/312,152, filed on Feb. 21, 2022; and U.S. Provisional Application No. 63/312,148, filed on Feb. 21, 2022, the disclosures of which are incorporated by reference herein in their entirety.

### BACKGROUND OF THE DISCLOSURE

### Field of the Disclosure

The present disclosure relates to systems and methods for packaging, distribution, storage, administration and/or disposal of a radiopharmaceutical (e.g., a radioactive drug used for therapy or imaging). The present disclosure further relates to systems and methods for packaging, distribution, storage, administration and/or disposal of therapeutic or diagnostic agents requiring precise volumetric delivery from a controlled source. In another embodiment, the present disclosure relates to systems, apparatuses, and methods for delivery and filtration of radiopharmaceuticals in medical injection procedures.

### Description of the Related Art

Radiopharmaceuticals can be utilized for targeted radionuclide therapy (TRT) or for diagnostic imaging. A radiopharmaceutical commonly includes a radioisotope (e.g., Ac-255, Lu-177, etc.), a targeting moiety or biovector (e.g., an antibody, a peptide, an antigen, small molecule, etc.), and optionally a chelator (e.g. DOTA, NOTA, DTPA, etc.) linked together into a single structure. In some cases, the TRT can be solely the radioisotope without a biovector or chelator when the radioisotope is one which the human body naturally takes up into tissues or organs. The radiopharmaceutical is configured to interact with a target protein on a cell, such as a cancer cell. The radiopharmaceutical can be mixed in a liquid form or a fluid form. In some examples or aspects, the radiopharmaceutical may be a solid particulate that is entrained in a fluid (e.g., a slurry that is suitable for injection into a patient). Administration is generally via intravenous administration into the systemic circulation.

Examples of TRTs can include targeted alpha therapies (TAT) or targeted beta therapies (TBT). Such therapies can be administered as mono therapies or in combination, such as via simultaneous or sequential administration. The radioactive therapeutic agent for TAT predominantly emits alpha radiation. The remainder of the emitted radiation from the radioactive therapeutic agent for TAT can include gamma radiation and/or beta radiation. A radioactive therapeutic agent for TBT predominantly emits beta radiation. The remainder of the emitted radiation for the radioactive therapeutic agent for TBT can include gamma radiation and/or alpha radiation. Examples of targeted alpha therapies include, without limitation, therapies based on thorium (Th-227) actinium (Ac-225), and lead (Pb-212). Examples of targeted beta therapies include therapies based on lutetium (Lu-177), copper (Cu-67), or iodine (I-131). Other examples of a radioactive therapeutic agents can include an alpha therapeutic agent that utilizes radium (Ra) (e.g. the Ra-223 isotope, such as the XOFIGO® treatment provided by Bayer Health Care). Processes for the preparation, prepared solutions, and use of XOFIGO® are described in U.S. Pat. No. 6,635,234, the disclosure of which is incorporated by reference herein in its entirety.

Radiopharmaceuticals used in TRT can pose significant production, storage, distribution, administration, handling, and disposal challenges. Because the therapeutic agent is radioactive, it can pose a radiation exposure to human health. Moreover, given the decaying nature of radioactive agents, the longer it takes to make, process, and deliver a radiopharmaceutical to a patient, the less activity is present in the administered dose. There are substantial regulations that must be followed to keep the radioactive material safely stored and utilized that can affect how the agent can be stored and transported as well as who may use or administer the radioactive therapeutic agent. For example, such regulations may require a care provider to have hundreds of hours of training in order to be able to administer any TRT.

FIG. 1 illustrates a conventional supply chain for a TRT. Initially, the radiopharmaceutical is manufactured in bulk at a manufacturing facility and is loaded into bulk containers. A shipment of such containers is delivered to a nuclear pharmacy, where a nuclear pharmacist draws a dose, for example into a syringe based on prescribed activity for a specific patient. That patient-ready dose is checked at the nuclear pharmacy in a dose calibrator to verify the prescribed dosing and assay. The dose is calibrated to the time of injection to assure that the dose has the necessary activity at the time of injection. The verified dose is then shipped to a treatment location, where it is again verified in a dose calibrator. At the treatment location, the dose usually has to be used within a certain number of hours of the dose draw before the dose is no longer suitable for patient use due to the half-life of the radioactive material. After administration, the used syringe is checked again in the dose calibrator to verify that the correct prescribed dose was administered to the patient.

As shown in FIG. 2, the process for diagnosing, referring, and treating a patient requires multiple processes and many different medical professionals. After a patient P is diagnosed by a doctor D, doctor D prescribes a dose of a radiopharmaceutical based on a dose regimen. That dose is filled by a nuclear pharmacist NP at a nuclear pharmacy before being delivered to an authorized user AU to verify the dose, administer the dose, and verify that the correct dose was delivered to the patient.

The conventional process for distribution and administration of TRT and other therapeutic or diagnostic agents that require precise volumetric delivery from a controlled source severely limits their applicability and use. After accounting for shipping, handling and patient scheduling, treatment locations will only have a limited time for administering a dose to a specific patient. The challenges imposed by variability in shipping, handling, and patient scheduling may have an impact on the efficacy of the TRT or other therapeutic or diagnostic agents, such as under-dosing at the time of delivery of the pharmaceutical to the patient. Due to these challenges associated with conventional systems and processes for distribution and administration of TRTs and other therapeutic or diagnostic agents that require precise volumetric delivery from a controlled source, there exists a need in the art for improved systems and processes for distribution, handling, administration, and disposal of such therapies.

Additionally, radiotherapy and radiology are rapidly expanding medical fields that utilize radioactive drugs for both treatment and imaging procedures. Delivering radiopharmaceuticals has several challenges. In some situations, radiation given off by the radiopharmaceutical may be dangerous to healthcare workers administering the drug to the patient. In some situations, daughter isotopes or products may be present in the radiopharmaceutical which may be beneficial to remove or reduce before delivery to a patient. An example of this is strontium breakthrough in a rubidium generator, as discussed in U.S. Pat. No. 8,071,959, the disclosure of which is hereby incorporated by reference in its entirety.

Furthermore, various forms of retention of the radiopharmaceutical in the patient can produce less than optimal treatment and/or adverse effects. Retention may include a condition in which the radiopharmaceutical is properly injected into the patient at a vascular, commonly venous access site, but due to various characteristics of the fluid flow and patient physiology, the radiopharmaceutical does not advance through the bloodstream as intended. This can result in a buildup of radioactive particles near the access site, or at other locations within the patient's venous structure. This may be termed stasis, slow clearance, or delayed clearance. Common causes include low blood flow in the vessel or adhesion of the radiopharmaceutical to the vessel wall. Extravasation, another type of retention, is a condition in which the radiopharmaceutical is inadvertently injected into the tissue outside of the target vasculature surrounding the injection site, and can likewise be dangerous to the patient.

Due to the above technical concerns as well as regulatory and training requirements, administration of radiopharmaceuticals is conventionally performed only by highly specialized healthcare workers, making such procedures less accessible than may be desired. Radiotherapy is heavily regulated due to its inherent challenges, and the regulations surrounding procedures are often complex. For example, dosages with an inaccuracy of 10% or more must be reported to appropriate regulatory agencies in some jurisdictions. Due to these challenges associated with conventional systems and processes for distribution and administration of radiopharmaceuticals and other therapeutic or diagnostic agents that require precise volumetric delivery from a controlled source, there exists a need in the art for improved systems and processes for distribution, handling, administration, and disposal of such therapies.

### SUMMARY OF THE DISCLOSURE

In view of the disadvantages of conventional systems and processes for distribution, handling, administration, and disposal of TRTs and other therapeutic or diagnostic agents, a better supply chain process is needed such that a treatment location can have TRTs available and ready to use for a longer period of time. Furthermore, improved systems and processes are needed to ensure that stored products are no longer patient specific. Instead, the treatment location can be provided equipment to help dose a treatment for any patient that may be in the location on any particular day so that there is more flexibility in how the stored product can be utilized at the treatment location such that an effective dose of the radiopharmaceutical can be delivered to the patient. Such patient-specific dosing is accomplished without the need for treatment location dose calibrators, thereby reducing or eliminating the need for manual measurements and handling within a designated hot lab. The dose, volume, and concentration may be accurately measured at the manufacturing or filling sites where it is much more efficient to use dosing and filling equipment, such as multiple dose calibrators with error detection and correction, automated handling of samples, automated recording of data, and accurate weighing or volume determination. The more accurate equipment and reduction or elimination of the chance for human error increases the reliability of the whole supply chain.

In some embodiments or aspects of the present disclosure, provided is a storage device configured to connect to a delivery system for delivering a therapeutic or diagnostic agent. The storage device may include: a housing having a chamber defined therein and a vessel positioned within the chamber. The vessel may have a distal end opposite a proximal end with an interior defined therebetween and configured for receiving the therapeutic or diagnostic agent. The proximal end of the vessel may have an access port for accessing the interior. The storage device further may have a door associated with the housing, the door being movable relative to the housing between a closed position and an open position. In the closed position, the door may cover an opening in the housing to enclose the chamber of the housing. In the open position, the door may reveal the opening in the housing for accessing the access port of the vessel. The storage device further may have a holder within the chamber of the housing and in contact with the vessel to fix the vessel relative to the housing such that the access port of the vessel is positioned at the opening in the housing. The door may be moveable between the closed position and the open position in response to actuation by an access mechanism of the delivery system.

In some embodiments or aspects of the present disclosure, the holder may include a contact element for contacting the distal end of the vessel and a plurality of tabs connected to the contact element and configured to engage an inner surface of the housing to fix the distal end of the vessel relative to the housing. The storage device further may include a plurality of ribs within the chamber of the housing and surrounding the opening. The plurality of ribs may be configured for fixing the proximal end of the vessel relative to the housing.

In some embodiments or aspects of the present disclosure, the storage device further may include a lock for locking the door in one of the open position and the closed position. A door cover may be connected to the housing, wherein the door cover encloses the door within a door chamber. The door cover may include a door access opening having a seal, and a vessel access opening, such as via a spike, positioned opposite the opening in the housing. The seal may be pierceable by the access mechanism of the delivery system.

In some embodiments or aspects of the present disclosure, the storage device further may include a label or a tag or data carrier on the housing that contains machine readable authenticatable data that includes at least one of product information, production information, prescription information, and shipping conditions information. The opening in the housing may be configured to receive a spike extending into the access port for accessing the therapeutic or diagnostic agent when the door is in the open position. In some embodiments or aspects, the therapeutic or diagnostic agent may be a radiopharmaceutical, and wherein the housing includes shielding configured to prevent radiation from the radiopharmaceutical from being emitted out of the housing.

In some embodiments or aspects of the present disclosure, provided is an assembly configured to connect to a delivery system for delivering a therapeutic or diagnostic agent. The assembly may include a storage device containing the therapeutic or diagnostic agent, and a fluid cassette fluidly connectable to the storage device for accessing the therapeutic or diagnostic agent. The storage device may include a housing having a chamber defined therein and a vessel positioned within the chamber. The vessel may have an interior configured for receiving therapeutic or diagnostic agent and an access port for accessing the interior. The storage device further may include a door associated with the housing, the door movable relative to the housing between a closed position and an open position. In the closed position, the door may cover an opening in the housing to enclose the chamber of the housing. In the open position, the door may reveal the opening in the housing for accessing the access port of the vessel. The fluid cassette may include a spike, a metering device, and a fluid path set fluidly connecting the spike to the metering device. The fluid cassette further may include an enclosure enclosing the spike, the metering device, and the fluid path set. The storage device and the fluid cassette may be configured to connect to a delivery system such that the door of the storage device is accessible by an access mechanism of the delivery system and such that the spike and the metering device of the fluid cassette are accessible by a delivery mechanism of the delivery system.

In some embodiments or aspects of the present disclosure, the spike of the fluid cassette may be insertable into access port of the vessel when the door is moved to the open position to fluidly connect the metering device to the vessel via the fluid path set. The fluid path set may include one or more valves operable by the delivery mechanism of the delivery system for regulating fluid flow through the fluid path element. The fluid cassette may be connectable to a saline source.

In some embodiments or aspects of the present disclosure, the storage device may include a guide mechanism configured for positioning the storage device in a desired orientation relative to the fluid cassette. The guide mechanism may include one or more geometric features on the storage device. The one or more geometric features may be configured to mate with corresponding one or more geometric features on the fluid cassette. The one or more geometric features may prevent mating between incompatible system components.

In some embodiments or aspects of the present disclosure, an outlet of the metering device of the fluid cassette may be configured to connect to an infusion set for delivering a dose of the therapeutic or diagnostic agent from the vessel to the infusion set. The storage device further may include a label or a tag on the housing that contains machine readable authenticatable data that includes at least one of product information, production information, prescription information, and shipping conditions information. The therapeutic or diagnostic agent may be a radiopharmaceutical, and wherein the housing includes shielding configured to prevent significant radiation from the radiopharmaceutical from being emitted out of the housing.

In some embodiments or aspects of the present disclosure, provided is a delivery system for delivering a therapeutic or diagnostic agent. The delivery system may include an injector having a delivery mechanism and an access mechanism, and a fluid delivery assembly removably connectable to the injector. The fluid delivery assembly may include a storage device containing the therapeutic or diagnostic agent, and a fluid cassette fluidly connectable to the storage device for accessing the therapeutic or diagnostic agent. The storage device may include a housing having a chamber defined therein, and a vessel positioned within the chamber. The vessel may have an interior configured for receiving therapeutic or diagnostic agent and an access port for accessing the interior. The storage device further may include a door associated with the housing, the door being movable relative to the housing via the access mechanism of the injector between a closed position and an open position. In the closed position, the door may cover an opening in the housing to enclose the chamber of the housing. In the open position, the door may reveal the opening in the housing for accessing the access port of the vessel. The fluid cassette may include a spike, a metering device, and a fluid path set fluidly connecting the spike to the metering device. The fluid cassette further may include an enclosure enclosing the spike, the metering device, and the fluid path set. The spike and the metering device of the fluid cassette may be accessible by the delivery mechanism of the injector for fluidly connecting the interior of the vessel with the metering device via the fluid path set.

In some embodiments or aspects of the present disclosure, the delivery system further includes an injector controller configured to determine a dose of the therapeutic or diagnostic agent to be drawn from the vessel into the metering device based on machine readable authenticated data on the storage device. The injector controller may be further configured to determine a dose of the therapeutic or diagnostic agent to be drawn from the vessel into the metering device based at least one patient parameter. The injector controller may be connected to a hospital network system, hospital enterprise system, or other healthcare network. The injector controller may include a plurality of dosing algorithms for different pre-defined therapies or diagnostic procedures.

In some embodiments or aspects of the present disclosure, the fluid path set may include one or more valves operable by the delivery mechanism of the delivery system for regulating fluid flow through the fluid path element. The fluid cassette may be connectable to a saline or other flushing fluid source. An outlet of the metering device of the fluid cassette may be configured to connect to an infusion set for delivering a dose of therapeutic or diagnostic agent from the vessel to the infusion set. The storage device may be configured to be removably or non-removably connectable to the fluid cassette.

In some embodiments or aspects of the present disclosure, provided is an inventory device for managing storage and disposal of used therapeutic or diagnostic agent. The inventory device may include a cart having a storage compartment that is accessible via a lockable door. The storage compartment may be configured to store one or more disposal containers. Each disposal container may include a storage device with a housing having a chamber defined therein, and a vessel positioned within the chamber of the housing. The vessel may be configured to store a radiopharmaceutical within an interior thereof. A door may be connected to the housing and be movable between an open position and a closed position. In the closed position, the door may entirely enclose the chamber of the housing. The device further may include a fluid cassette having a spike and a metering device. The storage device may be affixed to the fluid cassette such that the spike is inserted into the vessel to fluidly connect the metering device to the vessel. The metering device may be connected to an infusion set used for injecting a dose of the radiopharmaceutical. The infusion set, the storage device, and the fluid cassette may be retained within the disposal container.

In some embodiments or aspects of the present disclosure, the cart may include at least one indicator associated with the storage compartment to indicate whether any of the one or more disposal containers have been stored for a pre-selected storage time period so that a radioactive component of the used therapeutic or diagnostic agent has decayed to a pre-selected safety threshold level. The cart may include wheels having a wheel lock configured to prevent unauthorized or unintended movement of the cart. The wheel lock may be an electronic lock in communication with a controller. The wheel lock may be a mechanical lock having a key or other mechanical lock mechanism. The wheel lock may be operatively connected with the lockable door so that the wheels are unlocked and rollable only after the lockable door is unlocked.

In some embodiments or aspects of the present disclosure, provided is a process for manufacture and distribution of a therapeutic or diagnostic agent. The process may include filling a vessel with the therapeutic or diagnostic agent, positioning the vessel within a chamber of a storage device having a housing, closing the storage device so that the housing fully encloses the vessel within the chamber, shipping the storage device to an administration facility, opening a door of the storage device using an access mechanism of a delivery system, disinfecting an access port of the vessel using a disinfection mechanism of the delivery system, and accessing the therapeutic or diagnostic agent within the vessel via the access port using the delivery system.

In some embodiments or aspects of the present disclosure, accessing therapeutic or diagnostic agent may include piercing the access port using a spike of a cassette connected to the storage device. The process further may include reading a label or a tag on the storage device to determine at least one of product information, production information, prescription information, and shipping conditions information. The process further may include disinfecting the access port by emitting ultraviolet light or outputting a disinfecting material.

In some embodiments or aspects of the present disclosure, provided is a process for storing and disposing of used therapeutic or diagnostic agent. The process may include collecting a storage device that retains a vessel with a remaining portion of the therapeutic or diagnostic agent, a cassette to which the storage device is fluidly connected, and an infusion set for positioning in a disposal container. The process further may include placing a label, tag, or other indicia on the disposal container to indicate date of use; positioning the disposal container having the storage device, the cassette, and the infusion set therein into a storage compartment; and indicating the disposal container is safe to dispose of after a pre-selected decay time period has elapsed. The process further may include reading the label or other indicia to determine at least one of product information, production information, prescription information, and shipping conditions information.

In some embodiments or aspects of the present disclosure, provided is a process for delivering a dose of a therapeutic or diagnostic agent. The process may include inserting a therapeutic or diagnostic agent into a vessel; positioning the vessel within a chamber of a storage device having a housing; closing a door of the storage device so that the housing fully encloses the vessel within the chamber to shield radiation emitted by the radiopharmaceutical from being emitted out of the housing for transportation and storage of the radiopharmaceutical; determining a dose of the radiopharmaceutical for a patient based on manufacturing information of the radiopharmaceutical included with the storage device; and unlocking the door of the storage device to open the housing to access the radiopharmaceutical within the vessel and inject the determined dose into a patient.

In some embodiments or aspects of the present disclosure, accessing the therapeutic or diagnostic agent may include piercing the access port of the vessel using a spike of a cassette connected to the storage device. The process further may include reading a label or a tag on the storage device to determine at least one of product information, production information, prescription information, and shipping conditions information. The process further may include disinfecting an access port of the vessel. Disinfecting the access port may include emitting ultraviolet light or outputting a disinfecting material.

Additional embodiments or aspects of the systems and processes described herein are detailed in one or more of the following clauses:

Clause 1: A storage device configured to connect to a delivery system for delivering a therapeutic or diagnostic agent, the storage device comprising: a housing having a chamber defined therein; a vessel positioned within the chamber, the vessel having a distal end opposite a proximal end with an interior defined therebetween and configured for receiving the therapeutic or diagnostic agent, the proximal end having an access port for accessing the interior; a door associated with the housing, the door movable relative to the housing between a closed position and an open position, wherein, in the closed position, the door covers an opening in the housing to enclose the chamber of the housing, and wherein, in the open position, the door reveals the opening in the housing for accessing the access port of the vessel; and a holder within the chamber of the housing and in contact with the vessel to fix the vessel relative to the housing such that the access port of the vessel is positioned at the opening in the housing; wherein the door is moveable between the closed position and the open position in response to actuation by an access mechanism of the delivery system.

Clause 2: The storage device according to clause 1, wherein the holder comprises a contact element for contacting the distal end of the vessel and a plurality of tabs connected to the contact element and configured to engage an inner surface of the housing to fix the distal end of the vessel relative to the housing.

Clause 3: The storage device according to clause 1 or 2, further comprising a plurality of ribs within the chamber of the housing and surrounding the opening, wherein the plurality of ribs is configured for fixing the proximal end of the vessel relative to the housing.

Clause 4: The storage device according to any of clauses 1 to 3, further comprising a lock for locking the door in one of the open positon and the closed position.

Clause 5: The storage device according to any of clauses 1 to 4, further comprising a door cover connected to the housing, wherein the door cover encloses the door within a door chamber.

Clause 6: The storage device according to any of clauses 1 to 5, wherein the door cover comprises a door access opening having a seal, and a vessel access opening positioned opposite the opening in the housing.

Clause 7: The storage device according to clause 6, wherein the seal is pierceable by the access mechanism of the delivery system.

Clause 8: The storage device according to any of clauses 1 to 7, further comprising a label or a tag on the housing that contains machine readable authenticatable data that includes at least one of product information, production information, prescription information, and shipping conditions information.

Clause 9: The storage device according to any of clauses 1 to 8, wherein the opening in the housing is configured to receive a spike extending into the access port for accessing the therapeutic or diagnostic agent when the door is in the open position.

Clause 10: The storage device according to any of clauses 1 to 9, wherein the therapeutic or diagnostic agent is a radiopharmaceutical, and wherein the housing comprises shielding configured to prevent radiation from the radiopharmaceutical from being emitted out of the housing.

Clause 11: An assembly configured to connect to a delivery system for delivering a therapeutic or diagnostic agent, the assembly comprising: a storage device containing the therapeutic or diagnostic agent; and a fluid cassette fluidly connectable to the storage device for accessing the therapeutic or diagnostic agent, wherein the storage device comprises: a housing having a chamber defined therein; a vessel positioned within the chamber, the vessel having an interior configured for receiving the therapeutic or diagnostic agent and an access port for accessing the interior; and a door associated with the housing, the door movable relative to the housing between a closed position and an open position, wherein, in the closed position, the door covers an opening in the housing to enclose the chamber of the housing, and wherein, in the open position, the door reveals the opening in the housing for accessing the access port of the vessel, wherein the fluid cassette comprises: a spike, a metering device, and a fluid path set fluidly connecting the spike to the metering device; and an enclosure enclosing the spike, the metering device, and the fluid path set, and wherein the storage device and the fluid cassette are configured to connect to a delivery system such that the door of the storage device is accessible by an access mechanism of the delivery system and such that the spike and the metering device of the fluid cassette are accessible by a delivery mechanism of the delivery system.

Clause 12: The assembly according to clause 11, wherein the vessel access member of the fluid cassette is insertable into access port of the vessel when the door is moved to the open position to fluidly connect the metering device to the vessel via the fluid path set.

Clause 13: The assembly according to clause 11 or 12, wherein the fluid path set comprises one or more valves operable by the delivery mechanism of the delivery system for regulating fluid flow through the fluid path element.

Clause 14: The assembly according to any of clauses 11 to 13, wherein the fluid cassette is connectable to a saline source.

Clause 15: The assembly according to any of clauses 11 to 14, wherein the storage device comprises a guide mechanism configured for positioning the storage device in a desired orientation relative to the fluid cassette.

Clause 16: The assembly according to clause 15, wherein the guide mechanism comprises one or more geometric features on the storage device, and wherein the one or more geometric features are configured to mate with corresponding one or more geometric features on the fluid cassette.

Clause 17: The assembly according to any of clauses 11 to 16, wherein an outlet of the metering device of the fluid cassette is configured to connect to an infusion set for delivering a dose of the therapeutic or diagnostic agent from the vessel to the infusion set.

Clause 18: The assembly according to any of clauses 11 to 17, further comprising a label or a tag on the housing that contains machine readable authenticatable data that includes at least one of product information, production information, prescription information, and shipping conditions information.

Clause 19: The assembly according to any of clauses 11 to 18, wherein the therapeutic or diagnostic agent is a radiopharmaceutical, and wherein the housing comprises shielding configured to prevent radiation from the radiopharmaceutical from being emitted out of the housing.

Clause 20: A delivery system for delivering a therapeutic or diagnostic agent, the delivery system comprising: an injector having a delivery mechanism and an access mechanism; and a fluid delivery assembly removably connectable to the injector, the fluid delivery assembly comprising: a storage device containing the therapeutic or diagnostic agent; and a fluid cassette fluidly connectable to the storage device for accessing the therapeutic or diagnostic agent, wherein the storage device comprises: a housing having a chamber defined therein; a vessel positioned within the chamber, the vessel having an interior configured for receiving the therapeutic or diagnostic agent and an access port for accessing the interior; and a door associated with the housing, the door movable relative to the housing via the access mechanism of the injector between a closed position and an open position, wherein, in the closed position, the door covers an opening in the housing to enclose the chamber of the housing, and wherein, in the open position, the door reveals the opening in the housing for accessing the access port of the vessel, wherein the fluid cassette comprises: a vessel access member, a metering device, and a fluid path set fluidly connecting the vessel access member to the metering device; and an enclosure enclosing the vessel access member, the metering device, and the fluid path set, and wherein the vessel access member and the metering device of the fluid cassette are accessible by the delivery mechanism of the injector for fluidly connecting the interior of the vessel with the metering device via the fluid path set.

Clause 21: The delivery system according to clause 20, further comprising an injector controller configured to determine a dose of the therapeutic or diagnostic agent to be drawn from the vessel into the metering device based on machine readable authenticated data on the storage device.

Clause 22: The delivery system according to clause 21, wherein the injector controller is further configured to determine a dose of the therapeutic or diagnostic agent to be drawn from the vessel into the metering device based at least one patient parameter.

Clause 23: The delivery system according to clause 21 or 22, wherein the injector controller is connected to a hospital network system.

Clause 24: The delivery system according to any of clauses 21 to 23, wherein the injector controller comprises a plurality of dosing algorithms for different pre-defined therapies or diagnostic procedures.

Clause 25: The delivery system according to any of clauses 20 to 24, wherein the fluid path set comprises one or more valves operable by the delivery mechanism of the delivery system for regulating fluid flow through the fluid path element.

Clause 26: The delivery system according to any of clauses 20 to 25, wherein the fluid cassette is connectable to a saline source.

Clause 27: The assembly according to any of clauses 20 to 26, wherein an outlet of the metering device of the fluid cassette is configured to connect to an infusion set for delivering a dose of the therapeutic or diagnostic agent from the vessel to the infusion set.

Clause 28: The assembly according to any of clauses 20 to 27, wherein the storage device is configured to be removably or non-removably connectable to the fluid cassette.

Clause 29: The delivery system according to any of clauses 20 to 28, further comprising a label or a tag on the housing that contains machine readable authenticatable data that includes at least one of product information, production information, prescription information, and shipping conditions information.

Clause 30: The delivery system according to any of clauses 20 to 29, wherein the therapeutic or diagnostic agent is a radiopharmaceutical, and wherein the housing comprises shielding configured to prevent radiation from the radiopharmaceutical from being emitted out of the housing.

Clause 31: An inventory device for managing storage and disposal of used therapeutic or diagnostic agent, the inventory device comprising: a cart having a storage compartment that is accessible via a lockable door, the storage compartment configured to store one or more disposal containers, each disposal container comprising: a storage device comprising: a housing having a chamber defined therein; a vessel positioned within the chamber of the housing, the vessel configured to store a radiopharmaceutical within an interior thereof; a door connected to the housing, the door movable between an open position and a closed position, wherein, in the closed position, the door entirely encloses the chamber of the housing; and a fluid cassette comprising a vessel access member and a metering device, the storage device affixed to the fluid cassette such that the vessel access member is inserted into the vessel to fluidly connect the metering device to the vessel, the metering device being connected to an infusion set used for injecting a dose of the radiopharmaceutical, wherein the infusion set, the storage device, and the fluid cassette are retained within the disposal container.

Clause 32: The inventory device according to clause 31, wherein the cart comprises at least one indicator associated with the storage compartment to indicate whether any of the one or more disposal containers have been stored for a pre-selected storage time period so that a radioactive component of the used therapeutic or diagnostic agent has decayed to a pre-selected safety threshold level.

Clause 33: The inventory device according to clause 31 or 32, wherein the cart comprises wheels having a wheel lock configured to prevent unauthorized movement of the cart.

Clause 34: The inventory device according to clause 33, wherein the wheel lock is an electronic lock in communication with a controller.

Clause 35: The inventory device according to clause 33 or 34, wherein the wheel lock is a mechanical lock having a key or other mechanical lock mechanism.

Clause 36: The inventory device according to any of clauses 33 to 35, wherein the wheel lock is operatively connected with the lockable door so that the wheels are unlocked and rollable only after the lockable door is unlocked.

Clause 37: A process for manufacture and distribution of a therapeutic or diagnostic agent, the process comprising: filling a vessel with the therapeutic or diagnostic agent; positioning the vessel within a chamber of a storage device having a housing; closing the storage device so that the housing fully encloses the vessel within the chamber; shipping the storage device to an administration facility; opening a door of the storage device using an access mechanism of a delivery system; disinfecting an access port of the vessel using a disinfection mechanism of the delivery system; and accessing the therapeutic or diagnostic agent within the vessel via the access port using the delivery system.

Clause 38: The process according to clause 37, wherein accessing the therapeutic or diagnostic agent comprises piercing the access port using a vessel access member of a cassette connected to the storage device.

Clause 39: The process according to clause 37 or 38, further comprising reading a label or a tag on the storage device to determine at least one of product information, production information, prescription information, and shipping conditions information.

Clause 40: The process according to any of clauses 37 to 39, wherein disinfecting the access port comprises emitting ultraviolet light or outputting a disinfecting material.

Clause 41: A process for storing and disposing of used therapeutic or diagnostic agent, the process comprising: collecting a storage device that retains a vessel with a remaining portion of the therapeutic or diagnostic agent, a cassette to which the storage device is fluidly connected, and an infusion set for positioning in a disposal container; placing a label, tag, or other indicia on the disposal container to indicate date of use; positioning the disposal container having the storage device, the cassette, and the infusion set therein into a storage compartment; and indicating the disposal container is safe to dispose of after a pre-selected decay time period has elapsed.

Clause 42: The process according to clause 41, further comprising reading the label, tag, or other indicia to determine at least one of product information, production information, prescription information, and shipping conditions information.

Clause 43: A process for delivering a dose of a therapeutic or diagnostic agent, the process comprising: inserting a therapeutic or diagnostic agent into a vessel; positioning the vessel within a chamber of a storage device having a housing; closing a door of the storage device so that the housing fully encloses the vessel within the chamber to shield radiation emitted by the radiopharmaceutical from being emitted out of the housing for transportation and storage of the radiopharmaceutical; determining a dose of the radiopharmaceutical for a patient based on manufacturing information of the radiopharmaceutical included with the storage device; and unlocking the door of the storage device to open the housing to access the radiopharmaceutical within the vessel and inject the determined dose into a patient.

Clause 44: The process according to clause 43, wherein accessing the therapeutic or diagnostic agent comprises piercing the access port using a vessel access member of a cassette connected to the storage device.

Clause 45: The process according to clause 43 or 44, further comprising reading a label or a tag on the storage device to determine at least one of product information, production information, prescription information, and shipping conditions information.

Clause 46: The process according to any of clauses 43 to 45, further comprising disinfecting an access port of the vessel.

Clause 47: The process according to clause 46, wherein disinfecting the access port comprises emitting ultraviolet light or outputting a disinfecting material.

Clause 48: A storage device configured to connect to a delivery system, the storage device comprising: a housing having a chamber defined therein; a vessel positioned within the chamber, the vessel containing a radiopharmaceutical within an interior thereof, wherein the radiopharmaceutical is a therapeutically or prophylactically effective amount of a free metallic cation of the alkaline earth metal radium-223; a door associated with the housing, the door movable relative to the housing between a closed position and an open position, wherein, in the closed position, the door covers an opening in the housing to enclose the chamber of the housing, and wherein, in the open position, the door reveals the opening in the housing for accessing the access port of the vessel; and a holder within the chamber of the housing and in contact with the vessel to fix the vessel relative to the housing such that the access port of the vessel is positioned at the opening in the housing; wherein the door is moveable between the closed position and the open position in response to actuation by an access mechanism of the delivery system.

Clause 49: An assembly configured to connect to a delivery system for delivering a radiopharmaceutical, the assembly comprising: a storage device containing the therapeutic or diagnostic agent; and a fluid cassette fluidly connectable to the storage device for accessing the therapeutic or diagnostic agent, wherein the storage device comprises: a housing having a chamber defined therein; a vessel positioned within the chamber, the vessel containing a radiopharmaceutical within an interior thereof, wherein the radiopharmaceutical is a therapeutically or prophylactically effective amount of a free metallic cation of the alkaline earth metal radium-223; a door associated with the housing, the door movable relative to the housing between a closed position and an open position, wherein, in the closed position, the door covers an opening in the housing to enclose the chamber of the housing, and wherein, in the open position, the door reveals the opening in the housing for accessing the access port of the vessel, wherein the fluid cassette comprises: a vessel access member, a metering device, and a fluid path set fluidly connecting the vessel access member to the metering device; and an enclosure enclosing the vessel access member, the metering device, and the fluid path set, and wherein the storage device and the fluid cassette are configured to connect to a delivery system such that the door of the storage device is accessible by an access mechanism of the delivery system and such that the vessel access member and the metering device of the fluid cassette are accessible by a delivery mechanism of the delivery system.

Clause 50: A delivery system for delivering a therapeutic or diagnostic agent, the delivery system comprising: an injector having a delivery mechanism and an access mechanism; and a fluid delivery assembly removably connectable to the injector, the fluid delivery assembly comprising: a storage device containing the therapeutic or diagnostic agent; and a fluid cassette fluidly connectable to the storage device for accessing the therapeutic or diagnostic agent, wherein the storage device comprises: a housing having a chamber defined therein; a vessel positioned within the chamber of the housing, the vessel containing a radiopharmaceutical within an interior thereof, wherein the radiopharmaceutical is a therapeutically or prophylactically effective amount of a free metallic cation of the alkaline earth metal radium-223; and a door associated with the housing, the door movable relative to the housing via the access mechanism of the injector between a closed position and an open position, wherein, in the closed position, the door covers an opening in the housing to enclose the chamber of the housing, and wherein, in the open position, the door reveals the opening in the housing for accessing the access port of the vessel, wherein the fluid cassette comprises: a vessel access member, a metering device, and a fluid path set fluidly connecting the vessel access member to the metering device; and an enclosure enclosing the vessel access member, the metering device, and the fluid path set, and wherein the vessel access member and the metering device of the fluid cassette are accessible by the delivery mechanism of the injector for fluidly connecting the interior of the vessel with the metering device via the fluid path set, wherein the injector comprises an injector controller configured to determine a dose of the radiopharmaceutical based on manufacturing data attached to the housing of the storage device, the injector controller communicatively connected to the injector to control the injector for injecting the dose so that the injected dose that is received by a patient is the dose determined by the injector controller based on the manufacturing data attached to the housing of the storage device.

Clause 51: An inventory device for managing storage and disposal of used radiopharmaceutical, the inventory device comprising: a cart having shelving that is accessible via a lockable door, the shelving configured to store disposal containers, each disposal container comprising: a storage device comprising: a housing having a chamber defined therein; a vessel positioned within the chamber of the housing, the vessel configured to store a radiopharmaceutical within an interior thereof; a door connected to the housing, the door movable between an open position and a closed position, wherein, in the closed position, the door entirely encloses the chamber of the housing; and a fluid cassette comprising a vessel access member and a metering device, the storage device affixed to the fluid cassette such that the vessel access member is inserted into the vessel to fluidly connect the metering device to the vessel, the metering device being connected to an infusion set used for injecting a dose of the radiopharmaceutical, wherein the infusion set, the storage device, and the fluid cassette are retained within the disposal container, wherein the cart comprises indicators for the shelving to indicate which disposal containers have been stored for a pre-selected storage time period so that the radiopharmaceutical has decayed so radioactivity of the material is at or below a pre-selected safety threshold level.

Clause 52: A process for manufacture and distribution of a radiopharmaceutical for a targeted radionuclide therapy or a diagnostic imaging service, the process comprising: filling a vessel with a radiopharmaceutical for a TRT or the diagnostic imaging service, wherein the radiopharmaceutical is a therapeutically or prophylactically effective amount of a free metallic cation of the alkaline earth metal radium-223; positioning the vessel within a chamber of a storage device having a housing; closing the storage device so that the housing fully encloses the vessel within the chamber; shipping the storage device to an administration facility; opening a door of the storage device using an access mechanism of a delivery system; disinfecting an access port of the vessel using a disinfection mechanism of the delivery system; and accessing the radiopharmaceutical within the vessel via the access port using the delivery system.

Clause 53: A process for storing and disposing of radiopharmaceutical used in a targeted radionuclide therapy or a diagnostic imaging service, the process comprising: collecting a storage device that retains a vessel with a remaining portion of radiopharmaceutical, a cassette to which the storage device is connected, and an infusion set for positioning in a disposal container; placing a label, tag, or other indicia on the disposal container to indicate date of use; positioning the disposal container having the storage device, the cassette, and the infusion set therein into a storage compartment; and indicating the disposal container is safe to dispose of after a pre-selected decay time period has elapsed, wherein the radiopharmaceutical is a therapeutically or prophylactically effective amount of a free metallic cation of the alkaline earth metal radium-223.

Clause 54: A process for injecting a dose of a targeted radionuclide therapy or a diagnostic imaging service, the process comprising: inserting a radiopharmaceutical into a vessel, wherein the radiopharmaceutical is a therapeutically or prophylactically effective amount of a free metallic cation of the alkaline earth metal radium-223; positioning the vessel within a chamber of a storage device having a housing; closing a door of the storage device so that the housing fully encloses the vessel within the chamber to shield radiation emitted by the radiopharmaceutical from being emitted out of the housing for transportation and storage of the radiopharmaceutical; determining a dose of the radiopharmaceutical for a patient based on manufacturing information of the radiopharmaceutical included with the storage device; and unlocking the door of the storage device to open the housing to access the radiopharmaceutical within the vessel and inject the determined dose into a patient.

Clause 55: A radiopharmaceutical dosing injection system for a therapeutically or prophylactically effective amount of a free metallic cation of radium-223, according to any preceding clause.

Clause 56: An inventory device for the managing, storage and disposal of a therapeutically or prophylactically effective amount of a free metallic cation of radium-223, according to any preceding clause.

Clause 57: A process further including a therapeutically or prophylactically effective amount of a free metallic cation of radium-223, according to any preceding clause.

In view of the foregoing, there also exists a need for devices, systems, and methods for improved delivery and filtration of radiopharmaceuticals. Additionally, there exists a need for systems for delivery of radiopharmaceuticals that can safely be performed by a broader range of healthcare professionals than is presently possible or allowed by regulatory entities. Embodiments of the present disclosure may improve conventional radiopharmaceutical injection systems and methods in a multitude of ways. Embodiments of the present disclosure may reduce radiation exposure to patients and operators, and provide more awareness to the operator of the dosages, dosage rates, and associated radiation associated with an injection procedure. Embodiments of the present disclosure may mitigate adverse treatment outcomes by automatically and reliably detecting and correcting conditions such as faulty dosages, radiopharmaceutical retention, extravasation, system leaks, and fluid line occlusions. Further, embodiments of the present disclosure may automatically perform test injections to identify these conditions before delivering the full dose of radiopharmaceutical. Embodiments of the present disclosure may also minimize the number of component connections made or separate before, during, and/or after a procedure to reduce possible points of contamination and leakage. Embodiments of the present disclosure may effectively isolate and store radioactive waste for later disposal. Embodiments of the present disclosure may also alleviate supply chains concerns due to the manner in which the radiopharmaceuticals are supplied and removed from the system after use. As a corollary to improvements in patient outcomes, the advantages of the present disclosure may also reduce the burdens of hospital staff in complying with various regulatory burdens, as many regulatory concerns may be addressed automatically by embodiments of the present disclosure. Embodiments of the present disclosure may provide remote access to experienced and/or licensed personnel such as authorized users to enable remote personnel to oversee or activate the preparation and delivery of the drug to meet legal requirements. This may enable more sites and more operators to deliver the drug, thus expanding patient access to the drug. Embodiments of the present disclosure may also perform some of the record keeping and data analysis and exchange functions required. With this background in mind, embodiments of the present disclosure are directed to a fluid injector system.

In some embodiments or aspects of the present disclosure, the present disclosure is directed to various fluid injector delivery systems.

Additional embodiments or aspects of such fluid injector delivery systems described herein are detailed in one or more of the following clauses:

Clause 1′: A fluid injector delivery system comprising: at least one fluid reservoir comprising a first fluid reservoir configured for receiving a radiopharmaceutical; a radiation filter in fluid communication with the at least one fluid reservoir and configured for retaining radioactive particles from the radiopharmaceutical passing though the radiation filter; at least one sensor configured to detect radioactivity in at least one of the first fluid reservoir, the radiation filter, and a fluid path element in fluid communication with the radiation filter; and a controller in operative communication with the at least one sensor, the controller programmed or configured to: receive a radioactivity measurement from the at least one sensor; and determine, based on the radioactivity measurement, that an amount of radioactive particles in at least one of the first fluid reservoir, the radiation filter, and the fluid path element satisfies a predetermined threshold.

Clause 2′: The fluid injector delivery system of clause 1′, wherein the predetermined threshold comprises at least one of: a predetermined prescribed dosage of the radiopharmaceutical; a predetermined safe dosage of the radiopharmaceutical; a predetermined retention amount; a predetermined rate of change of radioactivity over volume of fluid moved through the filter; and a predetermined rate of change of radioactivity over time.

Clause 3′: The fluid injector delivery system of any of clause 1′ or 2′, wherein the controller is further programmed or configured to determine, based on the radioactivity measurement and one or more injection parameters, a cumulative amount of the radiopharmaceutical injected from the first fluid reservoir.

Clause 4′: The fluid injector delivery system of clause 3′, wherein the one or more injection parameters comprise at least one of: an injection rate of the radiopharmaceutical; an injection volume of the radiopharmaceutical; an injection duration of the radiopharmaceutical; a half-life of the radiopharmaceutical; a decay chain of the radiopharmaceutical; an age of the radiopharmaceutical; a total volume of the radiopharmaceutical; a concentration of the radiopharmaceutical; and an initial radioactivity of the radiopharmaceutical.

Clause 5′: The fluid injector delivery system of clause 3′, wherein the controller is further programmed or configured to compare the cumulative amount of the radiopharmaceutical injected to a predetermined prescribed dosage.

Clause 6′: The fluid injector delivery system of clause 3′, wherein the controller is further programmed or configured to adjust an injection rate of the radiopharmaceutical based on the cumulative amount of the radiopharmaceutical injected.

Clause 7′: The fluid injector delivery system of clause 3′, wherein the controller is further programmed or configured to halt an injection procedure in response to the cumulative amount of the radiopharmaceutical injected satisfying the predetermined threshold.

Clause 8′: The fluid injector delivery system of any of clause 1′-7′, wherein the controller is further programmed or configured to determine, based on the radioactivity measurement received from the at least one sensor, a residual level of radioactive particles in the radiation filter or in the fluid path set.

Clause 9′: The fluid injector delivery system of any of clause 1′-8′, wherein the controller is further programmed or configured to determine, based on the radioactivity measurement received from the at least one sensor, that the radiopharmaceutical is in chelation.

Clause 10′: The fluid injector delivery system of any of clause 1′-9′, wherein the at least one sensor comprises: a first sensor associated with the fluid path set upstream of the radiation filter; and a second sensor associated with the fluid path set downstream of the radiation filter, wherein the controller is programmed or configured to determine the amount of radioactive particles retained by the radiation filter by comparing a radioactivity measurement from the first sensor and a radioactivity measurement from the second sensor.

Clause 11′: The fluid injector delivery system of any of clause 1′-10′, wherein the fluid path set comprises an intermediate vessel in fluid communication with the at least one fluid reservoir and located downstream of the at least one fluid reservoir, wherein at least one of the at least one sensors is associated with intermediate vessel so as to detect radioactivity in the intermediate vessel, and wherein the controller is configured to prohibit delivery of the radiopharmaceutical from the intermediate vessel to a patient based on the amount of radioactive particles in the intermediate vessel deviating from the predetermined threshold.

Clause 12′: The fluid injector delivery system of any of clause 1′-11′, wherein the at least one fluid reservoir further comprises an additional fluid reservoir configured for injecting a flushing agent.

Clause 13′: The fluid injector delivery system of any of clause 1′-12′, wherein the at least one fluid reservoir further comprises an additional fluid reservoir configured for injecting another pharmaceutical.

Clause 14′: The fluid injector delivery system of clause 13′, wherein the another pharmaceutical is a protectant.

Clause 15′: The fluid injector delivery system of any of clause 1′-14′, wherein the first fluid reservoir comprises a radiopharmaceutical generator.

Clause 16′: The fluid injector delivery system of any of clause 1′-15′, wherein the first fluid reservoir comprising: a housing having a chamber defined therein; a vessel positioned within the chamber, the vessel having a distal end opposite a proximal end with an interior defined therebetween and configured for receiving the a radiopharmaceutical, the proximal end having an access port for accessing the interior; a door associated with the housing, the door movable relative to the housing between a closed position and an open position, wherein, in the closed position, the door covers an opening in the housing to enclose the chamber of the housing, and wherein, in the open position, the door reveals the opening in the housing for accessing the access port of the vessel; and a holder within the chamber of the housing and in contact with the vessel to fix the vessel relative to the housing such that the access port of the vessel is positioned at the opening in the housing, wherein the door of the first fluid reservoir is moveable between the closed position and the open position in response to actuation by an access mechanism of the fluid injector delivery system.

Clause 17′: A filtration system for a radiopharmaceutical fluid injector system, the filtration system comprising: a radiation filter in fluid communication with at least one fluid reservoir of the radiopharmaceutical fluid injector system, the radiation filter configured for retaining radioactive particles from a radiopharmaceutical received from the radiopharmaceutical fluid injector system; at least one sensor configured to detect radioactivity in at least one of the fluid reservoir, the radiation filter, and a fluid path set in fluid communication with the radiation filter; and a controller in operative communication with the at least one sensor, the controller programmed or configured to: receive a radioactivity measurement from the at least one sensor; and determine, based on the radioactivity measurement, that an amount of radioactive particles in at least one of the fluid reservoir, the radiation filter, and the fluid path set satisfies a predetermined threshold.

Clause 18′: A fluid injector system comprising: at least one fluid reservoir comprising a first fluid reservoir configured for injecting a radiopharmaceutical; a fluid path set in communication with the at least one fluid reservoir, the fluid path set comprising one or more fluid path elements including a catheter configured for insertion into a venous access site of a patient; a patient sensor configured to detect radioactivity in the patient in relation to the venous access site; and a controller in operative communication with the patient sensor and the fluid path set sensor, the controller programmed or configured to: receive radioactivity measurements from the patient sensor; and determine, based on the radioactivity measurements from the patient sensor, a presence or absence of retention of the radiopharmaceutical in the patient.

Clause 19′: The fluid injector system of clause 18′, further comprising a fluid

path set sensor configured to detect radioactivity in the fluid path set upstream of the venous access site.

Clause 20′: The fluid injector system of any of clause 18′ or 19′, wherein the controller is further programmed or configured to modify or halt an injection procedure in response to determining the presence of retention.

Clause 21′: The fluid injector system of any of clause 18′-20′, wherein the controller is further programmed or configured to determine, based on the radioactivity measurements from of the patient sensor and the fluid path set sensor, retention of the radiopharmaceutical in relation to the venous access site.

Clause 22′: The fluid injector system of any of clause 19′-21′, wherein the at least one fluid reservoir further comprises a second fluid reservoir configured for injecting a flushing agent, and wherein the controller is programmed or configured to increase the injection of the flushing agent in response to determining retention of the radiopharmaceutical in the venous access site.

Clause 23′: The fluid injector system of any of clause 19′-21′, wherein the controller is further programmed or configured to determine, based on a comparison of the radioactivity measurements from the patient sensor and the fluid path set sensor, a leakage in the fluid path set.

Clause 24′: The fluid injector system of any of clause 19′-21′, wherein the controller is further programmed or configured to determine, based on a comparison of the radioactivity measurements from the patient sensor and the fluid path set sensor, a blockage in the fluid path set.

Clause 25′: The fluid injector system of any of clause 18′-24′, further comprising a reference sensor configured to detect radioactivity in the patient remotely from the venous access site, wherein the controller is programmed or configured to: receive radioactivity measurements from the reference sensor; and determine, based on a comparison of the radioactivity measurements from the patient sensor and the reference sensor, the presence or absence of retention of the radiopharmaceutical into the patient.

Clause 26′: A retention detection system for a radiopharmaceutical fluid injector system, the retention detection system comprising: a patient sensor configured to detect radioactivity in the patient in proximity to a venous access site of a patient; a fluid path set sensor configured to detect radioactivity in a fluid path set of the fluid injector system upstream of the venous access site; and a controller in operative communication with the patient sensor and the fluid path set sensor, the controller programmed or configured to: receive radioactivity measurements from the patient sensor and the fluid path set sensor; and determine, based on the radioactivity measurements from the patient sensor and the fluid path set sensor, a presence or absence of retention of the radiopharmaceutical into the patient.

Further details and advantages of the various examples described in detail herein will become clear upon reviewing the following detailed description of the various examples in conjunction with the accompanying drawing figures.

### BRIEF DESCRIPTION OF THE DRAWINGS

FIG. 1 is a representative schematic of a conventional supply chain for a radioactive therapeutic agent configured for use with TRT according to the prior art;

FIG. 2 is a representative schematic of a conventional process for administration of TRT according to the prior art;

FIG. 3 is a representative schematic of an improved supply chain for a radioactive therapeutic agent configured for use with TRT according to some embodiments or aspects of the present disclosure;

FIG. 4 is a representative schematic of an improved process for administration of TRT according to some embodiments or aspects of the present disclosure;

FIG. 5 is a perspective view of a system for distribution, administration, and disposal of a liquid product requiring precise volumetric delivery from a controlled source in accordance with some embodiments or aspects of the present disclosure;

FIG. 6 is a perspective view of a system for distribution, administration, and disposal of a liquid product requiring precise volumetric delivery from a controlled source in accordance with some embodiments or aspects of the present disclosure;

FIG. 7 is a perspective view of a storage device for storing a liquid product, such as a radioactive therapeutic agent, in accordance with some embodiments or aspects of the present disclosure;

FIG. 8 is a cross-sectional perspective view of the storage device shown in FIG. 7;

FIG. 9 is an exploded top perspective view of the storage device shown in FIG. 7;

FIG. 10 is a cross-sectional perspective view of a storage device shown with a first vessel;

FIG. 11 is a cross-sectional perspective view of a storage device shown with a second vessel;

FIG. 12 is a detailed perspective view of a security cover on an access door of a storage device in accordance with some embodiments or aspects of the present disclosure;

FIGS. 13-14 are detailed views of a locking mechanism for preventing reuse of a storage device in accordance with some embodiments or aspects of the present disclosure;

FIG. 15 is a perspective view of a storage device for storing a liquid product, such as a radioactive therapeutic agent, in accordance with some embodiments or aspects of the present disclosure;

FIG. 16 is a side view of the storage device shown in FIG. 15;

FIGS. 17A-17B show bottom perspective views of storage devices in accordance with some embodiments or aspects of the present disclosure;

FIG. 18 is an exploded perspective view of the storage device shown in FIG. 15;

FIG. 19 is a cross-sectional perspective view of the storage device shown in FIG. 15;

FIG. 20 is a perspective view of a storage device and a fluid cassette for administering a dose from the storage device in accordance with some embodiments or aspects of the present disclosure;

FIG. 21 is a perspective view the fluid cassette shown in FIG. 20;

FIG. 22 is an exploded perspective view of the fluid cassette shown in FIG. 21;

FIG. 23 is a perspective view of a vessel access member configured to pierce a vessel of a storage device in accordance with some embodiments or aspects of the present disclosure;

FIG. 24 is a detailed perspective view of a metering device connection interface of a fluid cassette in accordance with some embodiments or aspects of the present disclosure;

FIG. 25A is a perspective view of a plunger cap shown in an unlocked position;

FIG. 25B is a perspective view of the plunger cap of FIG. 25A shown in a locked position;

FIG. 26 is a perspective view of a fluid cassette and components of a delivery system configured for interacting with the fluid cassette in accordance with some embodiments or aspects of the present disclosure;

FIG. 27 is a perspective view of a storage device and a fluid cassette for administering a dose from the storage device in accordance with some embodiments or aspects of the present disclosure;

FIG. 28 is a detailed view of a connection between the storage device and the fluid cassette shown in FIG. 27;

FIG. 29 is a perspective view the fluid cassette shown in FIG. 28;

FIG. 30 is an exploded perspective view of the fluid cassette shown in FIG. 29;

FIG. 31 is a schematic view of fluid connections between a vessel of a storage device, a fluid cassette, and a patient delivery line in accordance with some embodiments or aspects of the present disclosure;

FIG. 32 is a perspective view of a fluid cassette and a storage device along with components of an infusion system configured for interacting with the fluid cassette and the storage device in accordance with some embodiments or aspects of the present disclosure;

FIG. 33 is a perspective view of the components of an infusion system shown in FIG. 32;

FIG. 34 is a perspective view of a disinfection system for disinfecting a portion of a storage device in accordance with some embodiments or aspects of the present disclosure;

FIG. 35 is a perspective view of a carrier tray for transporting a plurality of storage devices in accordance with some embodiments or aspects of the present disclosure;

FIG. 36 is a perspective view of a disposal container for disposing a storage device and a cassette in accordance with some embodiments or aspects of the present disclosure;

FIG. 37 is a schematic view of a storage enclosure for storing a plurality of disposal containers in accordance with some embodiments or aspects of the present disclosure;

FIG. 38 is a flow diagram for a patency check procedure in accordance with some embodiments or aspects of the present disclosure;

FIG. 39 is a flow diagram for an administration procedure using a system described in accordance with some embodiments or aspects of the present disclosure;

FIG. 40 is a schematic of a fluid injector system according to an embodiment or aspect of the present disclosure;

FIG. 41 is a graph of radiation sensor output during an injection procedure performed by the system of FIG. 40;

FIG. 42 is a partial schematic of a fluid injector system according to an embodiment or aspect of the present disclosure;

FIG. 43 is a partial schematic of a fluid injector system according to an embodiment or aspect of the present disclosure;

FIG. 44 is a schematic of a fluid injector system according to an embodiment of the present disclosure;

FIG. 45 is a schematic of a fluid injector system according to an embodiment or aspect of the present disclosure;

FIG. 46 is a schematic of a fluid injector system according to an embodiment or aspect of the present disclosure;

FIG. 47 is a schematic of a fluid injector system according to an embodiment or aspect of the present disclosure;

FIG. 48a is a graph of fluid flow during an injection procedure performed by the system of FIG. 47;

FIG. 49 is a graph of radiation sensor output during the injection procedure of FIG. 48;

FIG. 50 is a graph of fluid flow during an injection procedure performed by the system of FIG. 47;

FIG. 51 is a graph of radiation sensor output during the injection procedure of FIG. 50;

FIG. 52 is a graph of fluid flow during an injection procedure performed by the system of FIG. 47;

FIG. 53 is a graph of radiation sensor output during the injection procedure of FIG. 52;

FIG. 54 is a schematic of a fluid injector system according to an embodiment or aspect of the present disclosure; and

FIG. 55 is a schematic of a remote control system that can be utilized in conjunction with any of the fluid injector systems of the present disclosure.

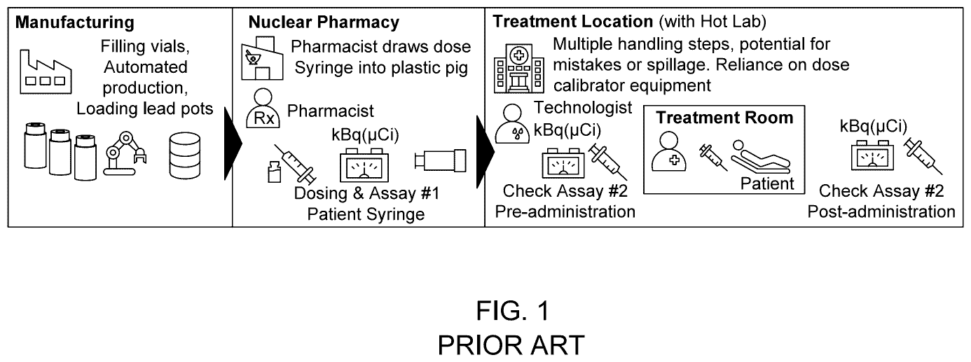

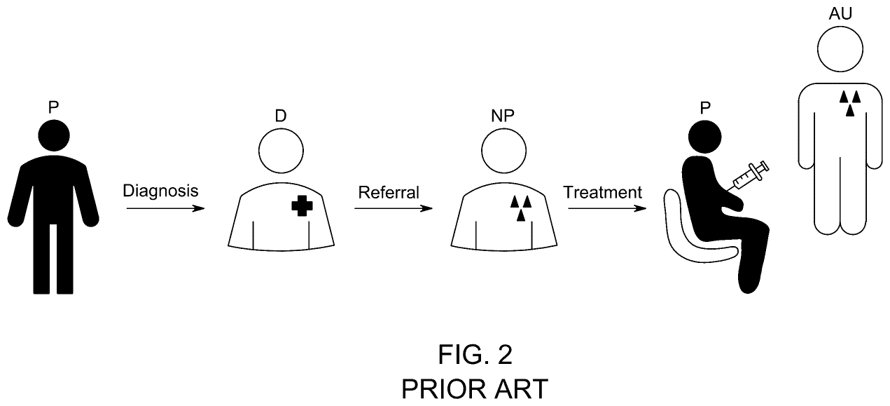

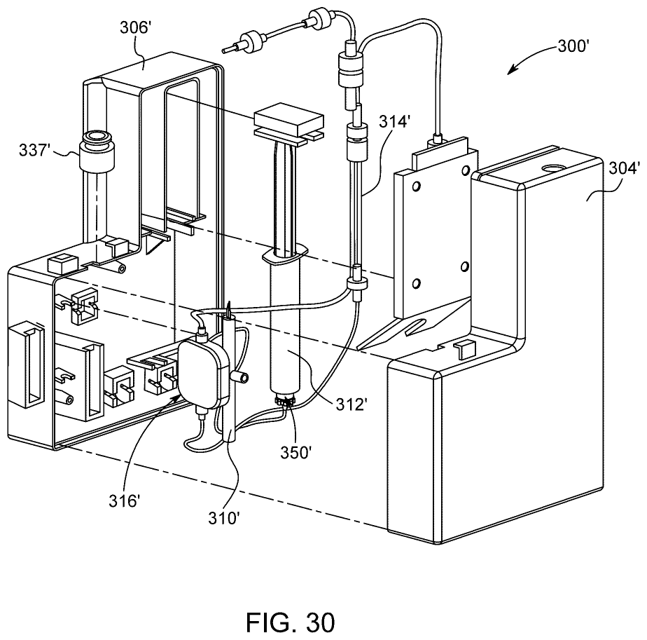

In FIGS. 1-55, like characters refer to the same components and elements, as the case may be, unless otherwise stated.

### DETAILED DESCRIPTION

As used herein, the singular form of “a”, “an”, and “the” include plural referents unless the context clearly dictates otherwise.

Spatial or directional terms, such as “left”, “right”, “inner”, “outer”, “above”, “below”, and the like, relate to the embodiments or aspects as shown in the drawing figures and are not to be considered as limiting as the embodiments or aspects can assume various alternative orientations.

All numbers used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less. The ranges and/or ratios disclosed herein represent the average values over the specified range and/or ratio.

The terms “first”, “second”, and the like are not intended to refer to any particular order or chronology, but refer to different conditions, properties, or elements.

All documents referred to herein are “incorporated by reference” in their entirety.

The term “at least” is synonymous with “greater than or equal to”.

The term “not greater than” is synonymous with “less than or equal to”.

Some non-limiting embodiments or aspects may be described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

As used herein, “at least one of” is synonymous with “one or more of”. For example, the phrase “at least one of A, B, or C” means any one of A, B, or C, or any combination of any two or more of A, B, or C. For example, “at least one of A, B, or C” includes A alone; or B alone; or C alone; or A and B; or A and C; or B and C; or all of A, B, and C.

The term “includes” is synonymous with “comprises”.

When used in relation to a component of a fluid delivery system such as a fluid reservoir, a syringe, or a fluid line, the term “distal” refers to a portion of said component nearest to a patient. When used in relation to a component of an injector system such as a fluid reservoir, a syringe, or a fluid line, the term “proximal” refers to a portion of said component nearest to the injector of the injector system (i.e. the portion of said component farthest from the patient). When used in relation to a component of a fluid delivery system such as a fluid reservoir, a syringe, or a fluid line, the term “upstream” refers to a direction away from the patient and towards the injector in relation to the normal flow of fluid of the injector system. When used in relation to a component of a fluid delivery system such as a fluid reservoir, a syringe, or a fluid line, the term “downstream” refers to a direction towards the patient and away from the injector in relation to the normal flow of fluid of the fluid delivery system.

As used herein, the terms “communication” and “communicate” may refer to the reception, receipt, transmission, transfer, provision, and/or the like, of information (e.g., data, signals, messages, instructions, commands, and/or the like).

The term “radiopharmaceutical”, as used herein, refers to a pharmaceutical comprising a radionuclide. Radiopharmaceuticals, as discussed herein, are preferably configured to be administered intravenously (i.v.). There are two types of radiopharmaceuticals: diagnostic (or imaging) and therapeutic radiopharmaceuticals, although in some instances, therapeutic radiopharmaceuticals may be used for both. For example, so TRSs may emit gamma radiation which may be used for dosimetry assessments and/or diagnostic purposes. Radiopharmaceutical generally used for imaging, such as the positron emitters, may also be used for therapy. See for example Hioki, T., Gholami, Y. H., McKelvey, K. J. et al. Overlooked potential of positrons in cancer therapy. Sci Rep 11, 2475 (2021).

The term “diagnostic radiopharmaceutical” or “imaging radiopharmaceutical”, as used herein, comprises Gamma emitting imaging radiopharmaceuticals for use in SPECT or SPECT/CT imaging and/or Positron emitting imaging radiopharmaceuticals for use in PET or PET/CT imaging. Examples of Gamma emitting imaging radiopharmaceuticals include, without limitation, technetium (Tc-99m), iodine (I-123), indium (In-111), gallium (Ga-67), or rhenium (Re-186). Examples of Positron emitting imaging radiopharmaceuticals include, without limitation, fluorine (F-18), gallium (Ga-68), zirconium (Zr-89), iodine (I-124), copper (Cu-64), rubidium (Rb-82) or yttrium (Y-86).

The term “therapeutic radiopharmaceutical”, as used herein, comprises Beta Therapeutic Radiopharmaceuticals, Alpha Therapeutic Radiopharmaceuticals, Positron Therapeutic Radiopharmaceuticals, Auger Therapeutic Radiopharmaceuticals, Gamma Therapeutic Radiopharmaceuticals, and/or combinations thereof.

As used herein, the term “therapeutic or diagnostic agent” refers to any diagnostic pharmaceutical, imaging pharmaceutical, a radiotherapy or chemotherapy pharmaceutical, a therapeutic pharmaceutical, or any other liquid or powder (once reconstituted) used in a therapeutic or diagnostic capacity that requires precise dose delivery from a controlled source, where dose is the amount of active ingredient. The dose delivery may be accomplished by precise volumetric delivery.

All radiation shielding is fractional or partial. Adding a half value layer of thickness to the shielding reduces the transmitted radiation by a factor of two. The effectiveness of shielding depends upon the energy of the radiation being shielded. Thus, terms such as “block”, “stop”, or “prevent” radiation transmission or radiation release indicate a reduction in transmitted or released radiation to an acceptable level. This acceptable level may be dependent upon local regulations, requirements, policies, or preferences. The many materials used for shielding and the guidelines involved are well known to those in the health physics field.

The disclosure comprises, consists of, or consists essentially of the following examples of the embodiments or aspects, in any combination. Various examples of the disclosure may be discussed separately. However, it is to be understood that this is simply for ease of illustration and discussion. In the practice of the disclosure, one or more aspects of the disclosure described in one example can be combined with one or more aspects of the disclosure described in one or more of the other examples.

In various embodiments or aspects, the present disclosure is directed to systems and processes for distribution, storage, administration, and disposal of a radiopharmaceutical therapeutic agent. The present disclosure is also directed to systems and processes for distribution, storage, administration, and disposal of other therapeutic or diagnostic agents that require precise volumetric delivery from a controlled source, such as chemotherapy pharmaceuticals. As discussed herein, conventional process for distribution and administration of therapeutic or diagnostic agents that require precise volumetric delivery from a controlled source severely limits their applicability and use. After accounting for shipping, handling and patient scheduling, treatment locations have a limited time for administering a dose to a specific patient. The systems and processes described herein provide an improvement in the distribution, storage, administration, and disposal of therapeutic or diagnostic agents to allow increased time for administering a dose to a specific patient.

As discussed in various embodiments or aspects of the present disclosure, a storage device can be configured to store a radioactive therapeutic agent from point of production for shipment and storage at a treatment facility so the radioactive therapeutic agent is fully enclosed and encased until use. Each of the storage devices can be sized, shaped, and configured to provide radiation shielding appropriate to the radioactive isotope and dose being stored. The storage devices can also be further packaged and surrounded by additional shielding. The storage device housing is configured to be opened and unsealed only at the treatment site utilizing a dedicated device, as described herein. In some embodiments or aspects, a storage device can be configured to store a therapeutic or diagnostic agent other than a radiopharmaceutical from point of production for shipment and storage at a treatment facility so the therapeutic or diagnostic agent is fully enclosed and encased until use.

As discussed in various embodiments or aspects of the present disclosure, a system can be provided to include a radioactive therapeutic agent injection/infusion system specifically adapted for accessing the therapeutic or diagnostic agent stored in a storage device so the material is only accessible for administering to a patient via the injection/infusion system and is otherwise prevented from being administered if the storage device is not recognized as an untampered storage device. The storage device can be configured so that only the injection/infusion system can open the storage device to access the therapeutic or diagnostic agent for injecting the agent into a patient. When the therapeutic or diagnostic agent is a radiopharmaceutical, the combination of the storage device and injection/infusion system can help ensure the radioactive material remains fully sealed and enclosed from time of production until use. Further, the injection/infusion system and storage device once used can remain connected and unopenable to facilitate safe containment and disposal of radioactive waste after use.

As discussed in various embodiments or aspects of the present disclosure, a system and process can be provided to calculate the dose for a specific patient based on the therapeutic or diagnostic agent being administered, the patient's parameters, such as patient weight, the known manufacturing and/or calibration date, known radioactivity or other property of the therapeutic or diagnostic agent at the time of manufacture, and known current date and time to determine the appropriate dose for the patient for injecting the volume of the therapeutic or diagnostic agent that corresponds to the calculated activity dose of the agent into the patient for the treatment. When the therapeutic or diagnostic agent is a radiopharmaceutical, the unused portion of the radiopharmaceutical and other components that contacted that radiopharmaceutical that may be contaminated can be stored in a disposal container for storage until the radioactivity has degraded to an acceptable level (usually about 10 half-lives of the radioactive isotope) depending upon the initial dose in the storage device. The system and process are configured such that dosing consistency can be assured in that each patient receives the desired amount of therapeutic or diagnostic agent.

As discussed in various embodiments or aspects of the present disclosure, improved systems and processes are provided for assuring that stored product is no longer designed to be or required to be patient specific. Instead, the systems disclosed herein are configured to dose a treatment for any patient that may be in the location on any particular day so that there is more flexibility in how the stored product can be utilized at the treatment location such that an effective dose of the radiopharmaceutical can be delivered to the patient. Such patient-specific dosing is accomplished without the need for dose calibrators, thereby eliminating additional dose assays for preparation of patient ready doses.

As discussed in various embodiments or aspects of the present disclosure, a system and process can be provided to monitor the therapeutic or diagnostic agent that is stored in the disposal container and indicate when the stored material has sufficiently decayed and is safe for throwing away. Once that determination is made, indication to the user can be provided (e.g. software prompts, or an LED light can turn from a red color to a green color or a red LED can be turned off and a green LED can be turned on) so staff can recognize that there is material that is suitable for disposal, locate the material to throw away, and suitably dispose of the material.

As discussed in various embodiments or aspects of the present disclosure, the improved inventory management flexibility associated with the systems and processes described herein permits care providers to more effectively manage inventory, which no longer has to be managed using a simplistic first in/first out approach and/or an approach that requires each stored dose to be provided for only a single, specific patient. Instead, inventory management and use of doses is managed to account for various factors including patient needs to better manage the supply of available doses. For instance, if a particular patient would require a large dose due to the patient's size (e.g. weight, height and weight, body composition, etc.), a newer, more radioactive vial of the therapeutic agent can be selected to administer to the patient so that only one vial (instead of multiple vials) is needed for the injection of a dose into the patient. This can permit the administration to occur more simply (e.g. only use of one injection sequence) and allow for more flexibility in terms of storage management and administration so doses are more efficiently utilized and less waste occurs. In some situations, this type of flexibility may also help reduce the exposure to a clinician during administering of the therapy and minimize the subsequent cleanup process after the patient has received his or her dose.

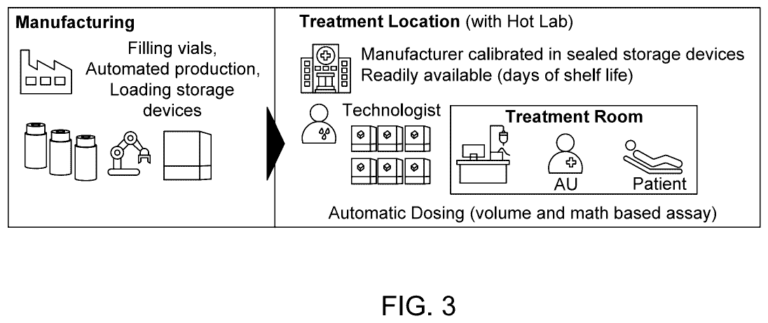

FIG. 3 illustrates an improved supply chain for a therapeutic or diagnostic agent in accordance with some embodiments or aspects of the present disclosure. The therapeutic or diagnostic agent can be manufactured in bulk at a manufacturing facility. Instead of loading the therapeutic or diagnostic agent into bulk containers for shipping to a hot lab, the therapeutic or diagnostic agent is loaded into storage devices which are shipped directly to a treatment location. The storage device is configured to store the therapeutic or diagnostic agent from the point of production, during shipping, and during storage at the treatment facility. The therapeutic or diagnostic agent is configured to be administered directly from the storage device to a patient using a delivery system, as described herein. Dosing for each specific patient is determined by the delivery system instead of using a dose calibrator.

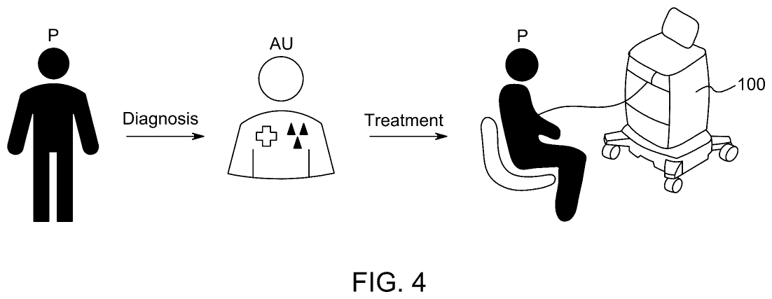

As shown in FIG. 4, the process for diagnosing, referring, and treating a patient in accordance with the improved supply chain eliminates multiple processes relative to a conventional process. After the patient P is diagnosed and prescribed a dose of the therapeutic or diagnostic agent based on the patient's weight, the dose can be administered using a delivery system 100 directly from a storage device 200 that is loaded into the delivery system 100. Prescribing and administration can be performed by the same authorized user AU.

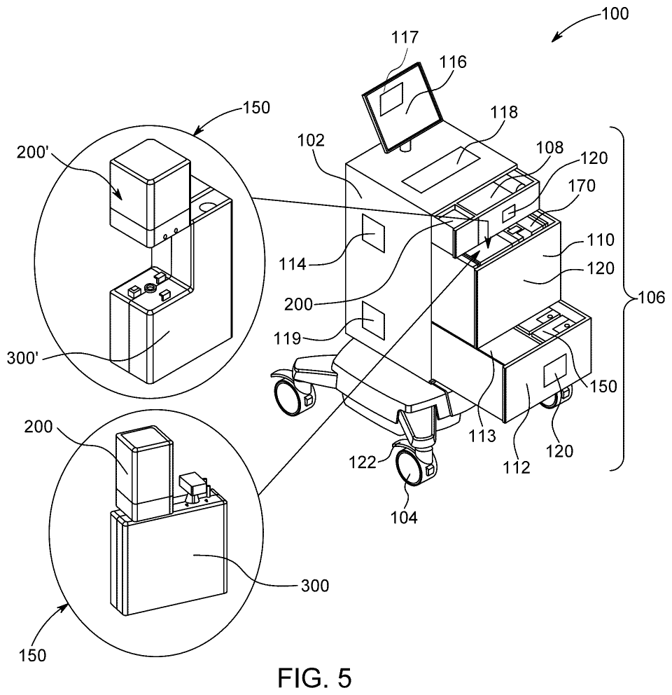

With reference to FIG. 5, the delivery system 100 for distribution, administration, and disposal of a therapeutic or diagnostic agent is shown in accordance with some embodiments or aspects of the present disclosure. As described herein, the system 100 includes a number of components that are designed to work together to provide a safe, streamlined, and flexible distribution of the therapeutic or diagnostic agent. The delivery system 100 is further configured to help end users maintain and manage an inventory of the therapeutic or diagnostic agent.

In some embodiments or aspects, the delivery system 100 can be configured to store, administer, and dispose of the therapeutic or diagnostic agent, such as a radiopharmaceutical therapeutic or diagnostic agent. The radiopharmaceutical therapeutic or diagnostic agent that can be stored and injected into a patient can be a material that is in a fluid. The radiopharmaceutical can emit primarily alpha radiation or can emit primarily beta radiation. In some embodiments or aspects, the radiopharmaceutical can emit primarily auger radiation or positron radiation and thus secondary gamma radiation. For material that may emit primarily beta radiation, the storage device 200 and the delivery system 100 can be adapted to address secondary X-ray radiation that can be emitted as a result of the shielding of the beta radiation, as described herein. The delivery system 100 can be adapted to address gamma radiation emitted additionally emitted by the radiopharmaceutical. In some examples or aspects, the delivery system 100 can be configured for storage, administration, and disposal of XOFIGO® treatments as well as other TRT treatments that may utilize targeted alpha therapy or a targeted beta therapy. The delivery system 100 may also be configured to work for radioisotopes that may be made at a treatment location for a treatment or a diagnostic service (e.g. a radioisotope with a very short half-life such as technetium-99 or copper-64 that can be used for imaging or other purposes).

In some embodiments or aspects, the delivery system 100 can be configured to store, administer, and dispose of the therapeutic or diagnostic agent that is a radiopharmaceutical, such as an imaging radiopharmaceutical. The imaging radiopharmaceutical may be, in some embodiments or aspects, a Gamma emitting imaging radiopharmaceutical. Gamma emitting imaging radiopharmaceuticals include, without limitation, 99mTc, 123I, 111In, 67Ga, and/or 186Re. In some embodiments or aspects, the imaging radiopharmaceutical may be a Positron emitting imaging radiopharmaceutical. Positron emitting imaging radiopharmaceuticals include, without limitation, 13N, 18F, 68Ga, 89Zr, 124I, 64Cu, 82Rb, and/or 86Y.

In some embodiments or aspects, the delivery system 100 can be configured to store, administer, and dispose of the therapeutic or diagnostic agent that is a radiopharmaceutical, such as a therapeutic radiopharmaceutical. The therapeutic radiopharmaceutical may be, in some embodiments or aspects, a Beta therapeutic radiopharmaceutical. Beta therapeutic radiopharmaceuticals include, without limitation, Lutetium-177, Iodine-131, Yttrium-90, Copper-67, Rhenium-188 and/or Holmium-166. The therapeutic radiopharmaceutical may be, in some embodiments or aspects, an Alpha therapeutic radiopharmaceutical. Alpha therapeutic radiopharmaceuticals include, without limitation, Radium-223, Actinium-225, Thorium-227, Astatine-211, Lead-212 and/or Bismuth-213. The therapeutic radiopharmaceutical may be, in some embodiments or aspects, an Auger therapeutic radiopharmaceutical. Auger therapeutic radiopharmaceuticals include, without limitation, Terbium-161 and/or Iodine-125. In some embodiments or aspects, the radiopharmaceutical is selected from the group consisting of 177Lu-Oxodotreotide, 223Ra dichloride, 18F-Fluciclovine, 123I-Ioflupane, 68Ga-Dotatate, 111In, 99mTc-Tilmanocept, 99mTc-tetrofosmin, 18F-Florbetaben, 99mTc, 90Y-Ibritumomab tiuxetan, 18F-Florbetapir, 153Sm-Lexidronam EDTMP, 131I-Iobenguane MIBG, 89Sr-Chloride. In some embodiments or aspects, the radionuclide of the radiopharmaceutical configured for imaging or therapy use is linked to FAP (fibroblast activation protein), PSMA (prostate specific membrane antigen), DOTA (dodecane tetraacetic acid and its chelating derivatives), HER2 (human epidermal growth factor receptor 2), GPC-3 (glypican-3 protein) or other mechanisms of action with radiopharmaceuticals.

In some embodiments or aspects, the delivery system 100 can be configured to work for radioisotopes that may be made at a treatment location for a treatment or a diagnostic service (e.g., a radioisotope with a very short half-life such as technetium-99m, nitrogen-13, flourine-18, gallium-68 or copper-64) that can be used for imaging or other purposes. These types of radioisotopes tend to have a very short half-life, which requires an imaging provider to prepare the radioisotopes in a hot lab or central radiopharmacy on site for effective use for a diagnostic service. For such an application, the provider may prepare the diagnostic service material on-site. The provider can then insert that material within a vial, place the vial in a storage device, and record and label that storage device to indicate its date of creation fluid volume, concentration, and/or initial radioactivity. The storage device may have a unique device identifier that is used to record the contents information in the software system. That storage device can then be coupled to a cassette, injector, and infusion set as described herein to provide a determined dose to a patient prior to the patient undergoing imaging. The used materials can also be stored etc. in a similar fashion for disposal, as described herein.

Following is a detailed description of various components of the delivery system 100 and how such components permit utilization of the delivery system 100 for an improved distribution, administration, and disposal therapeutic or diagnostic agents.

With continued reference to FIG. 5, the delivery system 100 is shown in accordance with one embodiment or aspect of the present disclosure. The delivery system 100 may be configured as a mobile device. The delivery system 100 includes a cart 102 supported on wheels 104 for moving the cart 102. In some embodiments or aspects, the delivery system 100 can be fixedly mounted. The cart 102 includes a plurality of storage components 106, such as shelves or drawers for storing various components of the delivery system 100. In some embodiments or aspects, the storage components 106 include a first drawer or shelf 108 for storing one or more storage devices 200 prior to use. The first drawer 108 may include the ability to keep a storage container 200 cold to meet the requirements of the drug being stored. The first drawer or shelf 108 may be further configured to store infusion sets and other fluid path components for connecting the delivery system 100 to a patient during administration of the therapeutic or diagnostic agent. The storage components 106 may include a second drawer or shelf 110 configured for receiving components of the delivery system 100 for administration of the therapeutic or diagnostic agent. For example, the second drawer or shelf 110 can be configured to receive one or more assemblies 150 for administering a dose of the therapeutic or diagnostic agent. As described herein, each assembly 150 includes a single storage device 200 connected to a single use fluid cassette 300. In some embodiments or aspects, the assembly 150 can include one or more multi-use storage devices 200 and multi-use fluid cassette 300. Each assembly 150 is removably insertable into the second drawer or shelf 110. The assembly 150 is operatively connectable to an injector 170 for delivering a dose of the therapeutic or diagnostic agent. The assembly 150 may be configured for a single use with a single patient. In some embodiments or aspects, the assembly 150 may be configured for use with multiple patients.

The delivery system 100 further includes a third drawer or shelf 112 configured for storing one or more used assemblies 150. Each assembly is configured to minimize the handling of the storage device 200 during workflow. At least one of the first, second, and third drawers or shelves 108, 110, 112 can have radiation shielding material to sufficiently reduce emission or radioactivity outside the cart 102.

With further reference to FIG. 5, the delivery system 100 further includes a controller 114 for controlling a delivery of a dose of the therapeutic or diagnostic agent. The controller 114 can be connected to one or more user displays 116 for displaying information relating to the storage, administration, and/or disposal aspects of the therapeutic or diagnostic agent. In some embodiments or aspects, the display 116 is a touch screen display enabling control via touch commands received from a user. The controller 114 can further be connected to an input device 118 for inputting data relating to storage, administration, and/or disposal of the therapeutic or diagnostic agent. In some embodiments or aspects, the input device 118 can be a bar code scanner, a keyboard, a mouse, a touch screen display, and/or any other input mechanism for inputting data and/or commands to the controller 114 relating to operation of the delivery system 100. The input device 118 may include the capability for video conferencing. The controller 114 can further be connected to a camera 117. The camera may be used to give input to the controller, such as reading a machine readable or human readable label or tag. The camera may be used to take images of, record, and/or communicate anything that is going on for use by the onsite operator, an offsite operator, or for training or archiving purposes. In some embodiments or aspects, an output device 119, such as a printer, is provided. The printer 119 may be used to print a label for documentation, a label to put on trash, a travel card for a patient, a reminder for patient, and/or guidelines for a patient.

The controller 114 may include at least one processor programmed or configured to calculate a dose of a therapeutic or diagnostic agent to be delivered to a specific patient based on patient data and/or data relating to one or more characteristics of the therapeutic or diagnostic agent. The at least one processor of the controller 118 further may be configured to actuate various components of the delivery system 100 to effect a delivery of a dose to a patient according to a programmed protocol for an injection procedure. The controller 118 may include computer readable media, such as memory, on which one or more injection protocols may be stored for execution by the at least one processor.

The controller 114 of the delivery system 100 can be adapted to determine a dose of the therapeutic or diagnostic agent to provide to a patient. The dose can be determined from one or more variables that can be provided to the controller 114 via the one or more input devices 118. For example, patient weight or other patient characteristics can be entered via a keyboard and/or mouse. Ranges of radiopharmaceutical activities of the therapeutic or diagnostic agent can be determined by the controller 114 based on information associated with the label or tag 270, such as a machine readable label (e.g. barcode) or electronic tag (e.g. RFID) attached to the storage device 200 shown in FIG. 16. In some embodiments or aspects, the information associated with the label or tag 270 can include manufacturing information and/or radioactivity information (e.g., date of manufacture, date and time of calibration, activity at calibration, level of radioactivity of the material at the time of manufacture, volume of fluid, concentration of fluid, type of radioisotope, etc.). In further embodiments or aspects, information associated with the label or tag 270 can include time and temperature history values of the storage device 200 during shipping and handling. This information as well as the current date or time can be used by the controller 114 to determine an appropriate dose for a patient by use of a pre-defined dosing algorithm that can utilize such parameters. Some pre-defined dosing algorithm can also use additional parameters, such as weight of the patient, sex of the patient, and/or age of the patient. Some pre-defined dosing algorithm can also use additional parameters such as prescribed dose and prescribed or targeted tissue dose.

In some embodiments or aspects, the controller 118 can have different dosing algorithms for different pre-defined treatments. The scanned barcode, read RFID tag, and/or other input provided by a user can be utilized to select the appropriate dosing algorithm to be run for determining the dose for the patient.

With continued reference to FIG. 5, each of the storage compartments 106 is lockable and is configured to be accessed by an authorized user having the appropriate access protocol. For example, each of the storage compartments 106 may have a lock 120 that is operatively connected to the controller 114. Operation of the lock 120 may require inputting a password or other authentication means using, for example, the display 116 or the input device 118 to authenticate an authorized user of the delivery system 100.

With continued reference to FIG. 5, one or more of the wheels 104 of the cart 102 may have a wheel lock 122 for selectively locking the wheels 104 to prevent movement of the cart 102. The wheel lock 122 may be configured to prevent unauthorized movement of the cart 102. For example, the wheel lock 122 may be operatively connected to the controller 114. Operation of the wheel lock 122 may require inputting a password or other authentication means using, for example, the display 116 or the input device 118 to authenticate an authorized user of the delivery system 100 to permit movement of the wheels 104. The wheel lock 122 may be a mechanical lock having a key or other mechanical lock mechanism. In some embodiments or aspects, the wheel lock 122 may be operatively connected with the lock 120 of the storage compartments 106 such that operation of one of the wheel lock 122 and the lock 120 also controls the operation of the other of the wheel lock 122 and the lock 120. In some embodiments or aspects, an alarm system may be operatively connected to at least one of the lock 120 and the wheel lock 122 such that an alarm may sound or an alarm message is provided for an unauthorized use of the delivery system 100. In some embodiments or aspects, the alarm system may be configured to prevent operation of the delivery system 100 and/or movement of the cart 102, such as by locking the wheel lock 122. Cart 102 may also include a compartment for storing ancillary equipment 113 which, while not used directly in the infusion process, is necessary or useful in the overall procedure. For example it may contain a survey meter or other radiation detector to survey the outside of the packaging and/or storage containers 200 when checking them in to the cart. The survey meter may also be used to survey the outside of assemblies 150 after an injector to check for leakage. The survey meter may also be used to survey the patient, the operator, and the injection room for any contamination, The auxiliary equipment compartment 113 may also contain a spill remediation kit for unusual instances where a spill occurs, for example if the IV comes out of the patient's arm during an infusion. The auxiliary equipment compartment may include other items commonly found in a hot lab and needed for this infusion because a benefit of this delivery system 100 is to provide the necessary capabilities and equipment to safely deliver the drugs it is designed to deliver.

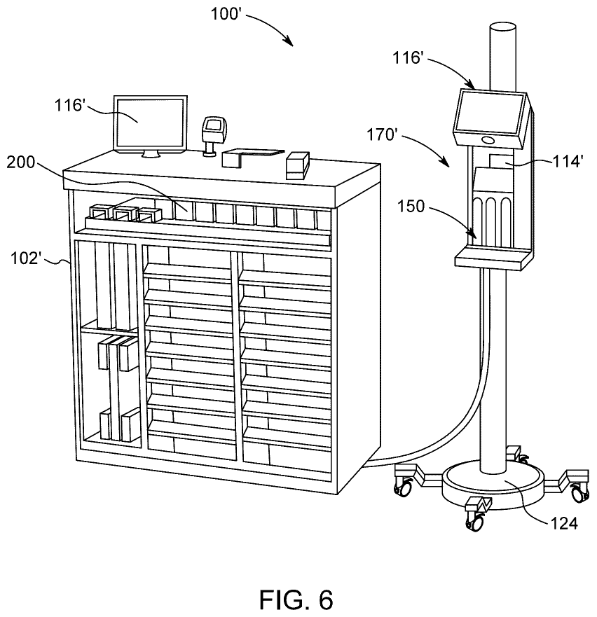

With reference to FIG. 6, a delivery system 100′ is shown in accordance with another embodiment or aspect of the present disclosure. Similar to the delivery system 100 shown and described with reference to FIG. 5, the delivery system 100′ shown in FIG. 6 is configured as a mobile device that includes a cart 102′ supported on optional wheels. The cart 102′ includes a plurality of storage components 106′ for storing one or more storage devices 200 prior to use and for storing one or more used assemblies 150.

Instead of incorporating the injector 170 into the cart 102, the delivery system 100′ shown in FIG. 6 has a separate injector 170′ supported on a separate movable base 124. The injector 170′ is configured to receive the assembly 150 including the storage device 200 and the fluid cassette 300. The delivery system 100′ further includes a controller 114′ for controlling a delivery of a dose of the therapeutic or diagnostic agent. The controller 114′ can be connected to one or more user displays 116′ for displaying information relating to the storage, administration, and/or disposal aspects of the therapeutic or diagnostic agent. In some embodiments or aspects, the display 116′ is a touch screen display enabling control via touch commands received from a user. The user display 116′ may be configured for inputting data relating to storage, administration, and/or disposal of the therapeutic or diagnostic agent. In some aspects some of the communications may be wireless so that no continuous physical connection is required between the selected parts. In some aspects of the system 100 there may be one or more fixed carts 102′ and one or more mobile carts 102 in communication with each other to facilitate flexible or optimal storage and use of the materials involved. In busy sites, used assemblies 150 may be transferred from movable carts to fixed carts which may provide auxiliary decay in place storage. This may be done, for example at the end of the day, either by moving individual units or through a transfer of a whole drawer from one cabinet to another. The auxiliary decay in place cabinet may be in a different room or even a different facility.

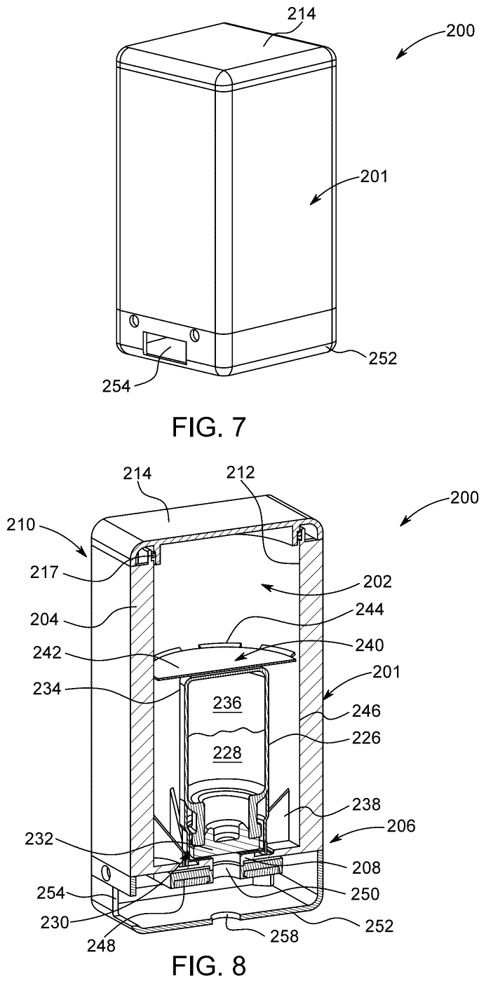

With reference to FIGS. 7-9, the storage device 200 is shown in accordance with one embodiment or aspect of the present disclosure. As discussed herein, the storage device 200 is configured to store a quantity of a therapeutic or diagnostic agent. In embodiments or aspects where the therapeutic or diagnostic agent is a radiopharmaceutical, the storage device 200 is configured to contain the radiation emitted by the radioisotope of the radiopharmaceutical.

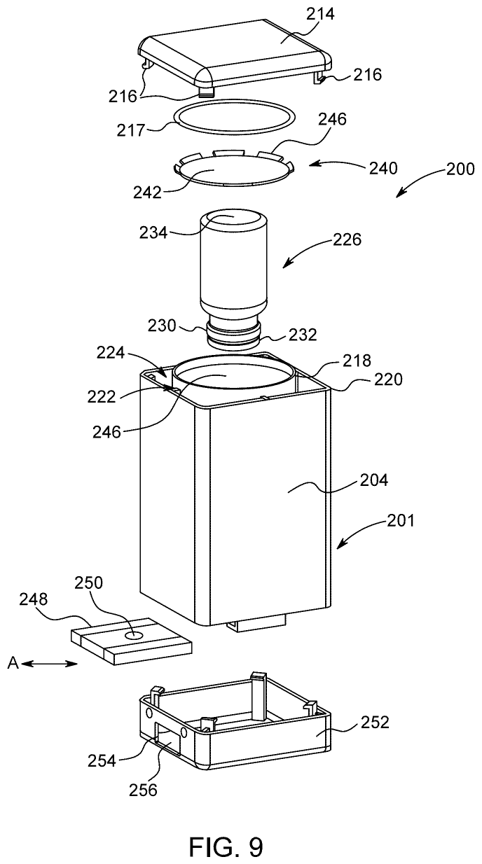

With reference to FIG. 8, the storage device 200 includes a housing 201 having a chamber 202 defined therein. The housing 201 has a main body 204 defining the chamber 202. The main body 204 has a proximal end 206 with a first opening 208 and a distal end 210 with a second opening 212. A cap 214 is provided to enclose the second opening 212 at the distal end 210 of the main body 204 of the housing 201. In some embodiments or aspects, a gasket or seal 217 is provided at an interface between the cap 214 and the distal end 210 of the main body 204. In some embodiments or aspects, the cap 214 is non-removably connected to the main body 204 via, for example, one or more clips 216 (shown in FIG. 9). In some embodiments or aspects, the cap 214 may be removably connectable to the main body 204. In some embodiments or aspects, the main body 204 and the cap 214 of the housing 201 may be made from, incorporate, or house a shielding material, such as poly(methyl methacrylate) (PMMA), lead, or tungsten.

With reference to FIG. 9, the main body 204 has an inner portion 218 defining the chamber 202 and an outer portion 220. The inner portion 218 may be connected to the outer portion 220 by one or more connectors 222. In some embodiments or aspects, a cavity 224 is defined between the inner portion 218 and the outer portion 220. The cavity 224 may be an air cavity, or it may be filled with one or more of a shielding material, such as PMMA, lead, or tungsten, a shock absorbing material such as polystyrene beads, or an absorbent material such as paper. The filling material may be formed for example from one or more of a solid sheet, in loose form, such as in the form of beads or pellets, a liquid, or a pourable filling that hardens, or a combination thereof. The inner portion 218 and the outer portion 220 may have the same shape or a different shape. For example, the inner portion 218 may have a substantially cylindrical shape while the outer portion 220 may have a substantially cuboid shape. The edges of the cuboid-shaped outer portion 220 may be rounded. In some embodiments or aspects, the inner portion 218 and the outer portion 220 may be monolithically formed.

For agents that emit beta radiation, the internal structure of the storage device 200 can be designed and configured to prevent X-rays formed from beta radiation from being emitted out of the housing 201. This blocking of beta radiation and X-rays can be affected by an inner structure of the storage device 200, such as a shielding material disposed in the cavity 224. Alternatively, or in addition, the sidewall of the housing 201 can be chosen, such as by selecting the thickness and material properties, to prevent an emissions of X-rays and beta radiation. In some embodiments or aspects, the storage device 200 can have a plurality of spaced shields (e.g. spaced apart shield walls) defined between the chamber 202 and the outer walls of the housing 201. In some configurations, packing or a fluid (e.g. air) can be positioned in the cavity 224 to provide sufficient shielding of X-rays and/or beta radiation.

Alpha radiation is typically not as difficult to block as alpha particles are large, have more limited penetrating power, and generally administered in lower doses than betas. The sidewalls of the housing 201 can be selected to be of a sufficient thickness for shielding the alpha radiation. Additional wall thickness or packing can be provided within the cavity 224 to help secure the vessel 226 in a desired location and/or provide additional shielding as many alpha and beta emitting isotopes or their daughter isotopes also give off gamma radiation to prevent any additional radiation exposure from the radiopharmaceutical being emitted out of the housing. The size of the housing 201 relative to the vessel 226 may be selected so as to set a minimum distance from the exterior of the housing 201 to the vessel 226 in order to reduce user exposure. In general, though, isotopes emit more than one type of radiation or radiation of different energy levels with different penetrating powers. In addition, all isotopes with have some buildup of daughter products between manufacture of the drug and delivery. Thus there may need to be significant gamma shielding for what are nominally alpha or beta emitters. The materials used for shielding and the guidelines involved are well known to those in the health physics field.

With reference to FIGS. 8-9, the chamber 202 of the housing 201 is configured to hold a vessel 226 containing a therapeutic or diagnostic agent 228 therein (shown in FIG. 8). The vessel 226 can be a glass vial that is formed separately from the housing 201 of the storage device 200 and is inserted into the chamber 202 of the housing 201. In some embodiments or aspects, the vessel 226 may be integrally formed with the storage device 200. Size of the chamber 202 is selected to accommodate the largest vessel 226 that may be used with the storage device 200 and/or to account for any additional packing or shielding material that may be required.

The vessel 226 has a proximal end 230 having an access port 232 and a closed distal end 234 with an interior 236 defined between the proximal end 230 and the distal end 234. The access port 232 may be a pierceable septum configured to be pierced by a vessel access member or other access mechanism for accessing the therapeutic or diagnostic agent 228 within the interior 236 of the vessel 226, as discussed herein. During manufacture of the therapeutic or diagnostic agent 228, the agent 228 can be filled into the vessel 226 and the vessel 226 can subsequently be sealed via the access port 232 to retain the agent 228 therein. Filling of the vessel 226 can occur after the vessel 226 is connected or positioned within a chamber 201 of the storage device 200, or prior to connecting or positioning the vessel 226 within the chamber 201. In some embodiments or aspects, the access port 232 may be fully sterilized at the point of manufacture. Vessel 226 may also be a plastic vial, a flexible bladder, a collapsible bag, or a prefilled syringe, preferably with a plunger but no handle to reduce the space needed. A benefit of collapsible vessels and prefilled syringes is that as fluid is pulled, the vessel collapses or plunger moves down so that no air needs to be admitted into the vessel as fluid is removed.

With reference to FIG. 8, the proximal end 230 of the vessel 226 is positioned at the proximal end 206 of the housing 201 such that the access port 232 is positioned opposite the first opening 208. In this manner, the vessel access member can extend through the first opening 208 of the housing 201 and through the access port 232 of the vessel 226 during administration of the therapeutic or diagnostic agent 228.

In some embodiments or aspects, the vessel 226 may be secured at the proximal end 206 of the housing 201 by a plurality of ribs 238 within the chamber 202 of the housing 201 and surrounding the first opening 208. Each of the plurality of ribs 238 may be configured to engage the proximal end 230 of the vessel 226 to fix the position of the access port 232 relative to the first opening 208 of the housing 201.

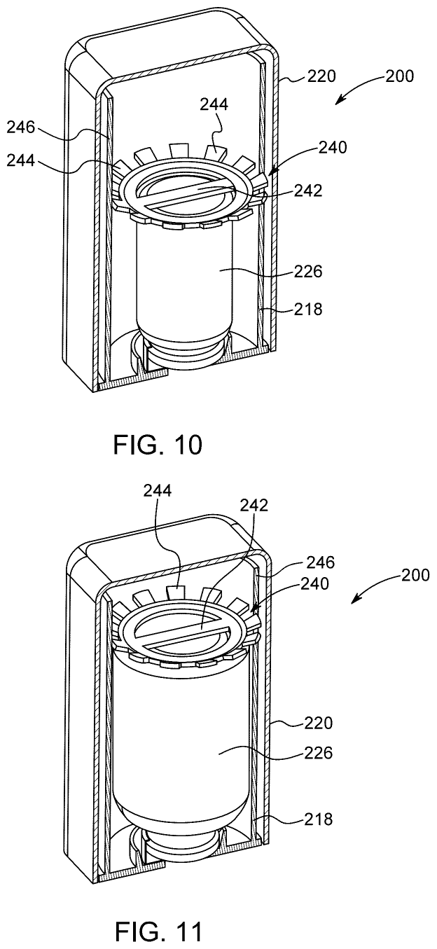

With reference to FIGS. 8-9, the storage device 200 has a holder 240 within the chamber 202 of the housing 201. The holder 240 may be configured to retain the distal end 234 of the vessel 226 relative to the housing 201. The holder 240 may be in contact with the distal end 234 of the vessel 226 so as to fix the position of the vessel 226 relative to the housing 201. In some embodiments or aspects, the holder 240 includes a contact element 242 for contacting the distal end 234 of the vessel 226 and a plurality of tabs 244 connected to the contact element 242 and configured to engage housing 201, such as an inner surface 246 of the inner portion 218 of the housing 201 to fix the position of the vessel 226 relative to the housing 201. As shown in FIGS. 9-10, the plurality of tabs 244 may be angled relative to the contact element 242 such that they are oriented at a non-perpendicular angle relative to the inner surface 246 of the inner portion 218 of the housing 201. The plurality of tabs 244 may flex relative to the contact element 242 such that, when the contact element 242 is pushed against the distal end 234 of the vessel 226, the plurality of tabs 244 provide a restoring force against movement of the holder 240 in a distal direction away from the distal end 234 of the vessel 226. In this manner, the holder 240 is configured to retain a plurality of different vessels 226, regardless of their diameter and longitudinal length.

With reference to FIGS. 8-9, the storage device 200 has a door 248 associated with the housing 201. In some embodiments or aspects, the door 248 may be movable relative to the housing 201 between a closed position and an open position shown in FIG. 8. The door 248 may be moveable between the closed position and the open position in response to actuation by an access mechanism of the delivery system 100, as described herein. In some embodiments or aspects, the door 248 may be normally closed. In the closed position, the door 248 is configured to cover the first opening 208 in the housing 201 to enclose the chamber 202 of the housing 201. In this manner, the vessel 226 is completely enclosed within the chamber 202 and access to the access port 232 is prevented. In the open position, the door 248 is moved relative to the housing 201 to reveal the first opening 208 in the housing 201 for accessing the access port 232 of the vessel 226. In some embodiments or aspects, the door 248 may be slidably movable relative to the housing in a direction of arrow A shown in FIG. 9.

With continued reference to FIGS. 8-9, the door 248 has an access aperture 250 configured to line up with the first opening 208 of the housing 201 when the door 248 is in the open position. In this manner, the vessel access member can extend through the access aperture 250 and the first opening 208 and into the access port 232 of the vessel 226.

With continued reference to FIGS. 8-9, a door cover 252 is connected to the housing 201 and is configured to enclose the door 248 within a door chamber. The door cover 252 is non-removably connected to the main body 204 via, for example, one or more clips 216 (shown in FIG. 9). In some embodiments or aspects, the door cover 252 may be removably connectable to the main body 204 of the housing 201. In some embodiments or aspects, door cover 252 may be made from the same material as the main body 204 and the cap 214 of the housing 201.

With reference to FIG. 9, the door cover 252 has a door access opening 254 having a seal 256 and a vessel access opening 258 (shown in FIG. 8) positioned opposite the first opening 208 of the housing 201. The vessel access opening 258 is configured to receive a spike or other vessel access member of the delivery system during administration of the therapeutic or diagnostic agent 228. The vessel access opening 258 is aligned with the access aperture 250 on the door 248 when the door 248 is in the open position to permit the spike or other vessel access member to extend through the first opening 208 on the housing 201 for access to the access port 234.

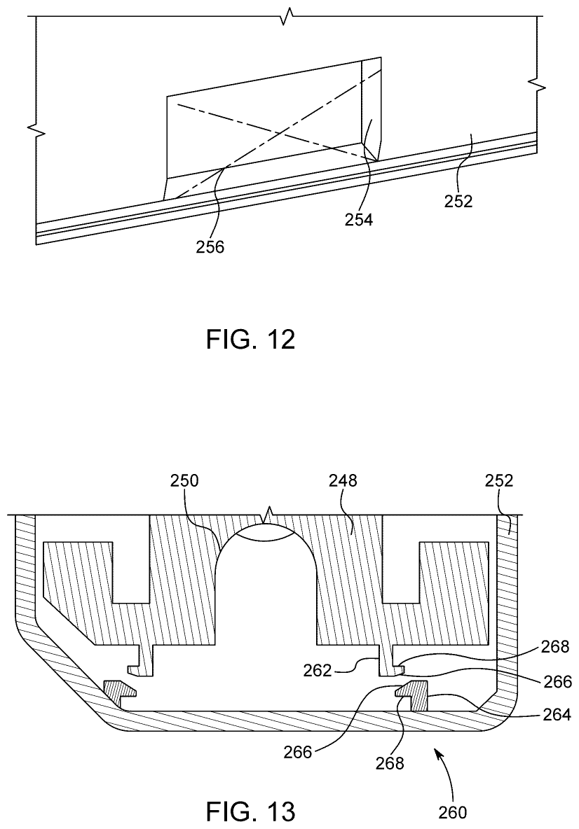

With reference to FIG. 12, the seal 256 covers the door access opening 254 and is pierceable by an access mechanism of the delivery system 100, as described herein. In some embodiments or aspects, the access mechanism of the delivery system 100 may be configured to sense the presence of the seal 256, such as by sensing a resistance to movement through the door access opening 254 when the seal 256 is present. If the access mechanism of the delivery system 100 does not detect the seal 256, such as due to no resistance to movement through the door access opening 254, the controller 114 may be configured to prevent operation of the delivery system 100 because a used storage device 200 (i.e., one with a pierced seal 256), or a tampered storage device 200 (i.e., one with a removed seal 256) has been installed for use with the delivery system 100. In this manner, the seal 256 functions as a security mechanism to assure that only an untampered storage device 200 can be used with the delivery system 100. In some embodiments or aspects, the seal 256 may also be provided over the vessel access opening 258. In some embodiments, the seal may be a member of the housing 201 which is broken off or permanently deformed as evidence of use or tampering.

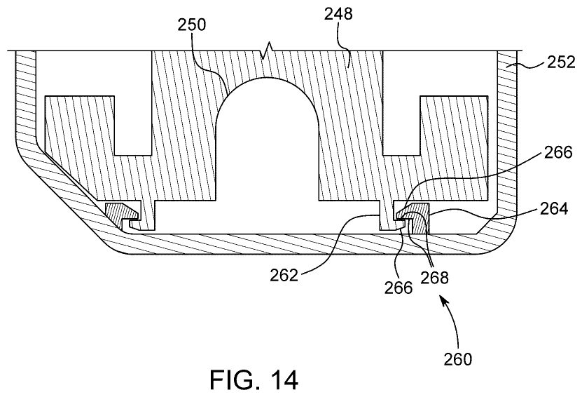

With reference to FIGS. 13-14, a door lock 260 is shown in accordance with some embodiments or aspects. The door lock 260 may be provided on the door 248 for locking the door 248 in the open position after the door 248 is moved from the closed position to the open position. In some embodiments or aspects, the door lock 260 includes at least one first hook 262 that is configured to engage with at least one second hook 264 on the housing 201 or the door cover 252 (shown in FIGS. 8-9). Each of the at least one first hook 262 and the at least one second hook 264 may have an angled contact surface 266 and a catch 268 that is configured to engage once the two contact surfaces 266 slide past each other. FIG. 14 shows the at least one first hook 262 and the at least one second hook 264 in a locked engagement with each other when the door 248 is in the open position. Due to this locked engagement, the door 248 cannot be moved back to the closed position.

In some embodiments or aspects, the door 248 may be movable between three different positions. In an initial position, the door 248 may be closed. In an intermediate position, the door 248 may be moved from the initial (closed) position to an open position to permit access to the vessel 226. In a final position, the door 248 may be moved to a closed position, in which the door 248 is engaged with the door lock 260 to prevent the door 248 from being reopened and any remaining contents in the vessel 226 from being accessed.

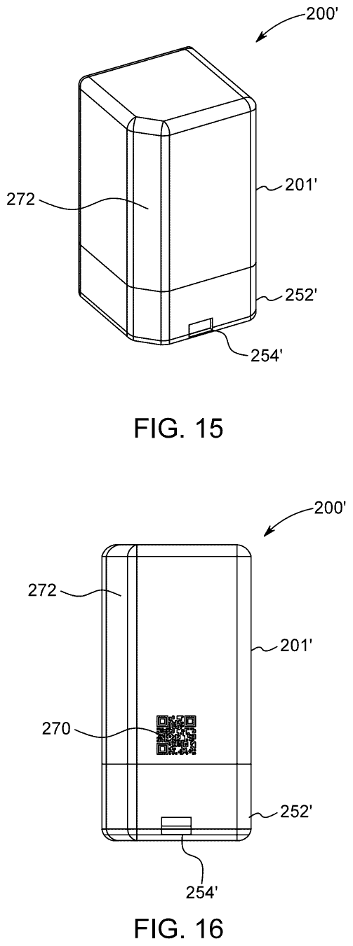

With reference to FIGS. 15-19, a storage device 200′ is shown in accordance with another embodiment or aspect of the present disclosure. As the structure of the storage device 200′ shown in FIGS. 15-19 is substantially similar to the structure of the storage device 200 shown and described with reference to FIGS. 7-14, a detailed description of the components of the storage device 200′ will be omitted. Same reference numbers will be used in FIGS. 15-19 to describe the components of the storage device 200′ as used in FIGS. 7-14 to describe the components of the storage device 200, except for the addition of a “′” mark after each reference number in FIGS. 15-19. The following detailed disclosure will focus only on relative differences between the two storage devices.

In some embodiments or aspects, the storage device 200 may have at least one label, tag, or other indicia 270 on the housing 201. While the at least one label, tag, or other indicia 270 is shown in connection with the embodiment of the storage device shown in FIG. 16, the at least one label, tag, or other indicia 270 may be applied to any storage device 200 described herein, such as the storage device 200 described herein with reference to FIGS. 7-14. The at least one label, tag, or other indicia 270 may contain machine readable authenticatable data is configured to be read by the delivery system 100 to authenticate the storage device 200 prior to use. In some embodiments or aspects, the machine readable authenticatable data includes at least one of product information, production information, prescription information, and shipping conditions information. The at least one label, tag, or other indicia 270 can be a label (e.g. barcode, a QR code, or similar) and/or a tag (e.g. electronic, RFID, or similar) that includes at least one of product information, production information, prescription information, and shipping conditions information relating to the therapeutic or diagnostic agent. In some embodiments or aspects, the at least one label, tag, or other indicia 270 can include a data logger configured for logging, for example, temperature, shock, and/or pressure data. In some embodiments or aspects, the at least one label, tag, or other indicia 270 can include a link to accessing information from a website or a database.

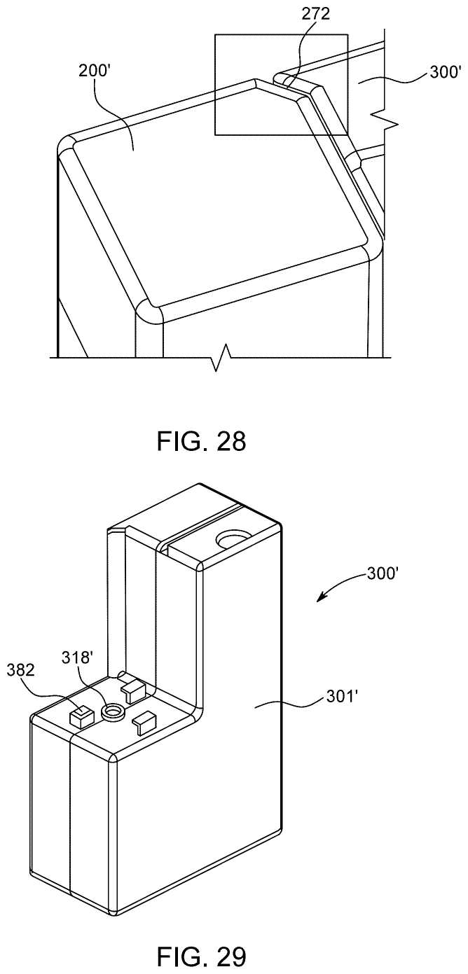

In some embodiments or aspects, the storage device 200′ can be configured such that the storage device 200′ is connectable to a fluid cassette only in a particular orientation. In this manner, the access opening 254′ on the door cover 252′ of the storage device 200′ can be properly aligned with the fluid cassette for proper connection of the vessel access member with the access port 232′ on the vessel 226′. With reference to FIGS. 15-17B, the housing 201′ of the storage device 200′ includes a guide mechanism 272 that is configured for positioning the storage device 200′ in a desired orientation relative to the fluid cassette 300 (see FIG. 28, for example). In some embodiments or aspects, the guide mechanism 272 includes one or more geometric features such as grooves, chamfers, projections, holes or tabs, etc. that can be configured to mate with corresponding features of the fluid cassette 300 for providing a direct connection to the fluid cassette 300 in a pre-determined orientation.

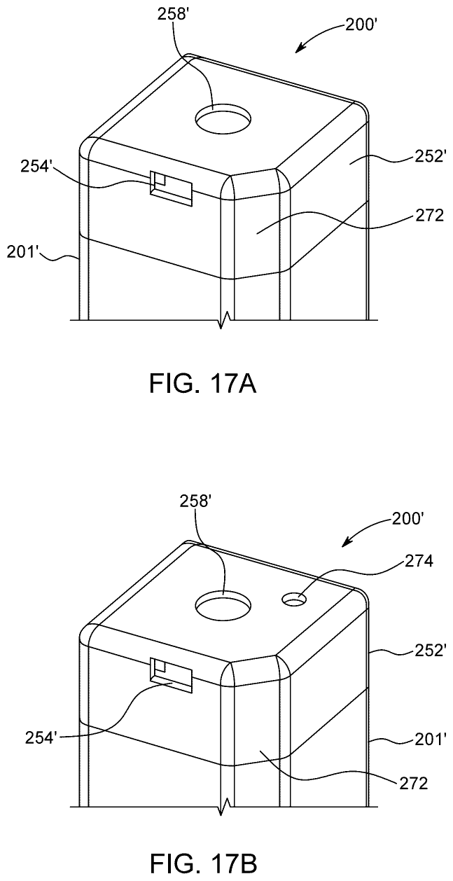

With reference to FIGS. 17A-17B, the storage device 200′ may have at least one identification feature 274 (shown in FIG. 17B) that may be used to identify a particular characteristic of the storage device 200′, such as the type of therapeutic or diagnostic agent in the storage device 200′. The at least one identification feature 274 may be one or more geometric features or physical indicia such as grooves, chamfers, projections, holes or tabs, etc. Such geometric features can be used to identify a particular storage device 200′ and/or verify that the storage device 200′ is an authentic storage device 200′. The at least one identification feature 274 may have a corresponding identification feature on the fluid cassette 300). The same or different identification features 274 may be used in relation to other aspects of the system, for example the storage compartment 108 and the used container compartment 112. This provides tangible feedback that the drugs in the storage containers 200′ are compatible with the shielding and temperature capabilities of the applicable storage compartments.

In some embodiments or aspects, at least a portion of the housing 201′ of the storage device 200′ may have a colored portion that may be used for identifying purposes. For example, a particular color on at least a portion of the housing 201′ of the storage device 200′ may be used for identifying the contents of the storage device 200′, such as the type of the therapeutic or diagnostic agent contained therein. In some embodiments or aspects, a particular color on at least a portion of the housing 201′ of the storage device 200′ may be used for identifying the storage device 200′ as a storage device 200′ intended for training purposes only. Such a storage device 200′ would not contain any therapeutic or diagnostic agent but a safe, harmless liquid, optionally with coloring so that its behavior can be visualized.

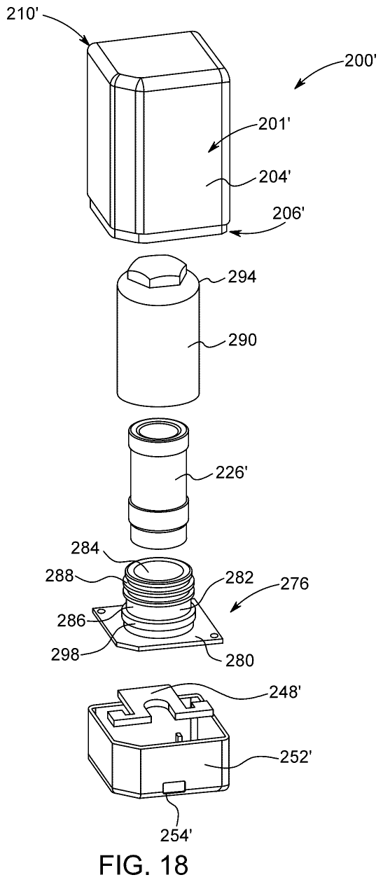

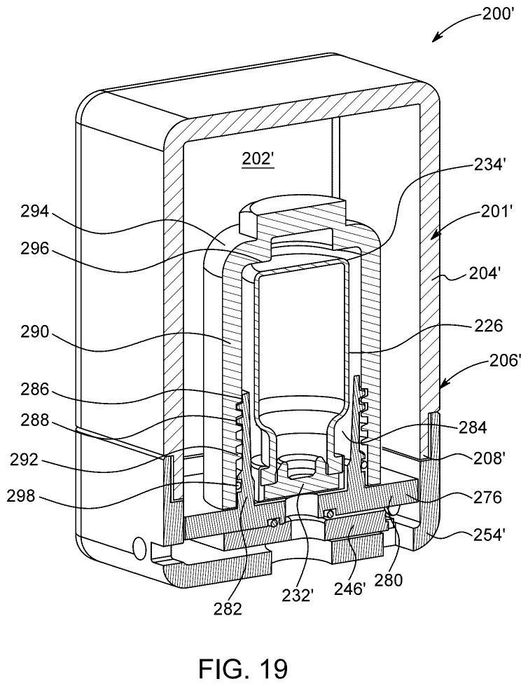

With reference to FIGS. 18-19, the storage device 200′includes a housing 201′ having a chamber 202′ defined therein (shown in FIG. 19). The housing 201′ has a main body 204′ with a proximal end 206′ with a first opening 208′ and a closed distal end 210′. Instead of having a cap 214 at the distal end 210, such as shown in the storage device 200 in FIGS. 7-9, the storage device 200′ shown in FIGS. 18-19 has a proximal cap 276 that is configured to enclose the first opening 208′ at the proximal end 206′ of the housing 201′.

With continued reference to FIGS. 18-19, the proximal cap 276 has a retainer 278 having a base 280 connectable to at least one of the main body 204′ and the door cover 252′ and a retaining portion 282 protruding distally from the base 280. The retaining portion 282 has a substantially cylindrical shape having an inner surface 284 configured to engage the vessel 226′ and an outer surface 286 having a threaded collar 288. The threaded collar 288 is configured to threadably engage with a cover 290 that encloses the vessel 226′ within the chamber 202′. The cover 290 has threads 292 configured to threadably engage with the threaded collar 288 on the retaining portion 282. A distal end 294 of the cover 290 has an inner engagement surface 296 configured to contact the distal end 234′ of the vessel 226′. A seal 298 may be provided at an interface between the retaining portion 282 and the cover 290.

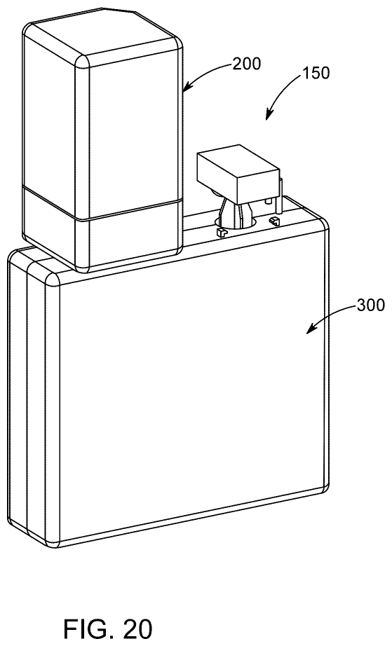

With reference to FIG. 20, an assembly 150 having a storage device 200 and a fluid cassette 300 is shown in accordance with some embodiments or aspects of the present disclosure. As described herein, the assembly 150 is configured to deliver the therapeutic or diagnostic agent from the storage device 200 via the fluid cassette 300 using the injector 170 (shown in FIG. 5). The fluid cassette 300 is configured to be removably connectable to the injector 170 and may be connected to a saline source for a saline flush application, priming, a saline test injection, and a saline infusion. The fluid cassette 300 is further configured to connect to the vessel 226 (shown in FIG. 8) of the storage device 200 to deliver the therapeutic or diagnostic agent 228 to a patient using the injector 170.

In some embodiments or aspects, the storage device 200 and the fluid cassette 300 may be configured to be removably connectable to each other. In other embodiments or aspects, the storage device 200 and the fluid cassette 300 may be configured to be non-removably connectable to each other, such that when the storage device 200 is connected to the fluid cassette 300, the storage device 200 cannot be removed from the fluid cassette 300. This type of interlocked connection helps prevent any undesired contact with the therapeutic or diagnostic agent. Each fluid cassette 300 can be adapted for connection to one storage device 200 or a pair of storage devices 200. In further embodiments or aspects, the fluid cassette 300 may only be fluidly connectable to the storage device 200, without any direct physical connection between housings thereof.

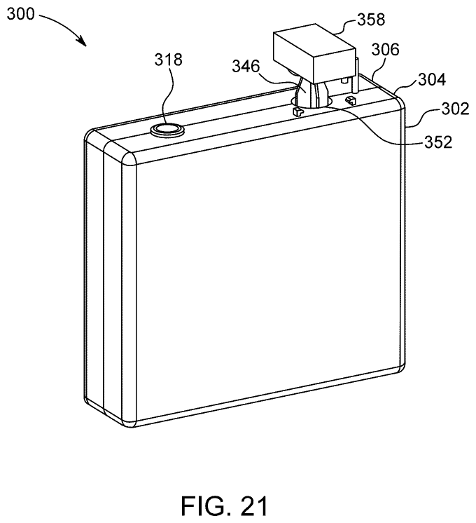

With reference to FIG. 21, the fluid cassette 300 is shown without the storage device shown in FIG. 20. The fluid cassette 300 includes an enclosure 302 that encloses various components of the fluid cassette 300. The enclosure 302 has a first portion 304 and a second portion 306. The first and second portions 304, 306 may be removably or non-removably connectable to each other. In some embodiments or aspects, the fluid cassette 300 may have a substantially cuboid shape.

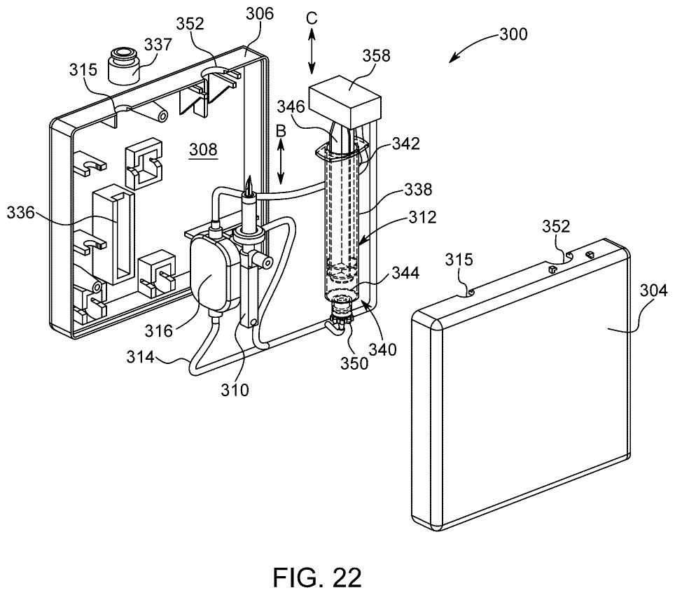

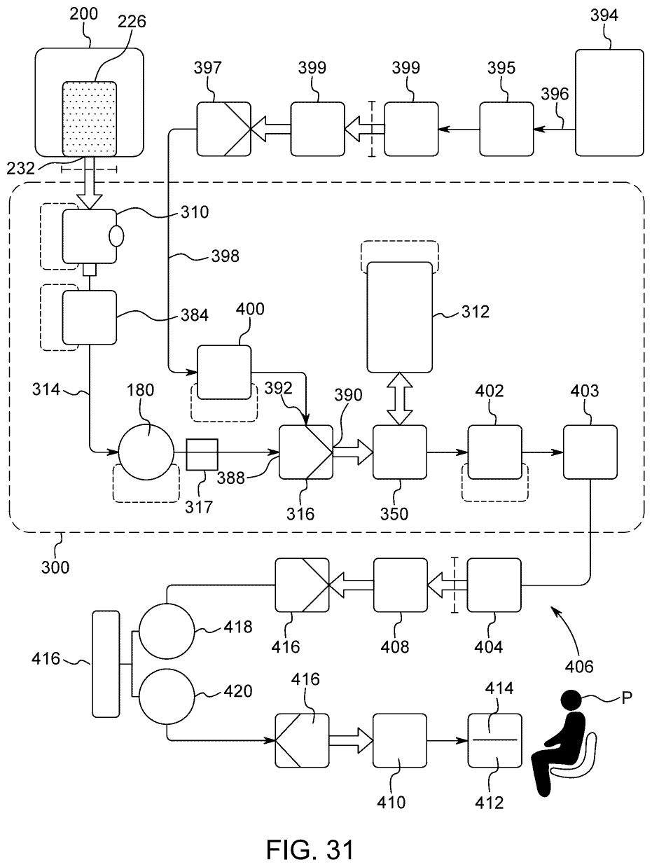

With reference to FIG. 22, the first and second portions 304, 306 of the enclosure 302 of the fluid cassette 300 define an interior 308 configured to receive components of the fluid cassette 300. In some embodiments or aspects, the fluid cassette 300 may have a vessel access member, such as a spike 310, a metering device, such as a syringe 312, and a fluid path set 314 received within the interior 308 of the enclosure 302. The fluid path set 314 has tubing that fluidly connects the spike 310 to the syringe 312. In some embodiments or aspects, the fluid path set 314 may have a particle/air filter 317 (shown in FIG. 31). In further embodiments or aspects, the fluid path set 314 may have a valve block, as described herein with reference to FIG. 31. In some embodiments, the valve block may have one or more valves to control fluid flow through the spike 310, the syringe 312, and the fluid path set 314. The fluid path set 314 is further configured for connecting to an infusion set, to be described herein with reference to FIG. 31.

With continued reference to FIG. 22, the spike 310 is configured to be extendable through a spike opening 315 in the enclosure 302 between a retracted position and an extended position in a direction of arrow B. In the retracted position, the spike 310 is contained within the interior 308 of the enclosure 302. In the extended position, the spike 310 protrudes from interior 308 of the enclosure 302 through the spike opening 315 such that the spike 310 is insertable through the access port 232 of the vessel 226 (shown in FIG. 8). An alignment element 337 may be provided on the spike opening 315 of the fluid cassette 300 for aligning the storage device 200 relative to fluid cassette 300 such that the spike 310 is axially aligned for insertion into the access portion 232 of the vessel 226 when the storage device 200 is connected to the fluid cassette 300. A cap may be provided for enclosing the spike opening 315 prior to use of the fluid cassette 300.

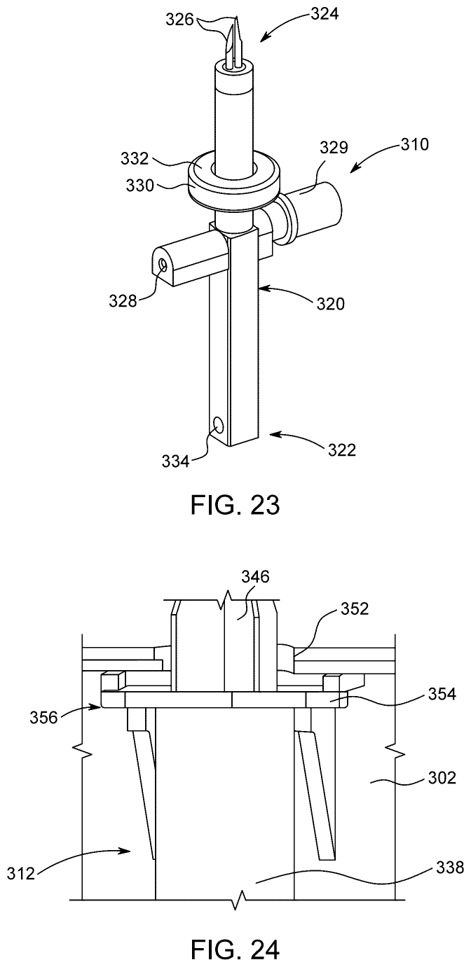

As shown in FIG. 23, the spike 310 has a body 320 with a proximal end 322, a distal end 324, and with a hollow interior. The distal end 324 of the spike 310 has at least one piercing tip 326 configured for piercing the access port 232 of the vessel 226 (shown in FIG. 8). The at least one piercing tip 326 may include two piercing tips 326, with a first of the two piercing tips 326 being configured to withdraw fluid from the vessel 226, and the second of the two piercing tips 326 being configured to deliver air into the vessel 226 as the fluid is withdrawn therefrom. The at least one piercing tip 326 is in fluid communication with the hollow interior of the body 320 of the spike 310 to deliver fluid from the access port 232 of the vessel 226 to a fluid path connector 328, which is adapted to connect to the fluid path set 314. In this manner, the therapeutic or diagnostic agent from the vessel 226 can be delivered to the fluid path set 314 via the at least one piercing tip 326 and the fluid path connector 328 of the spike 310. In some embodiments or aspects, the spike may have a filtered vent 329 configured to allow air to enter the vessel 226 as fluid is withdrawn from the vessel 226. The spike 310 may have a collar 330 extending around the body 320. In some embodiments or aspects, an absorbent material 332 may be provided on the collar 330 for absorbing any drips from the access port 232 as the spike 310 is inserted into the access port 232 or withdrawn from the access port 232. The spike 310 further has a drive element 334 configured for engagement with a spike drive of the delivery system, as described herein. The drive element 334 may be an opening, slot, or other feature configured to be engaged by the spike drive of the delivery system for moving the spike 310 between the retracted position and the extended position. As shown in FIG. 22, a spike drive slot 336 may be provided on the enclosure 302 such that the spike drive of the delivery system can extend into the interior 308 of the enclosure 302 to engage the spike 310.

With reference to FIG. 22, the syringe 312 has barrel 338 with a proximal end 340 opposite a distal end 342 and an interior chamber 344 defined therebetween. The proximal end 340 is open and is configured to receive a plunger 346. The distal end 342 has a port 350 in fluid communication with the fluid path set 314. The plunger 346 is reciprocally movable within the barrel 338 of the syringe 312 via a syringe drive of the delivery system, as described herein. The plunger 346 is movable is a direction of arrow C, wherein movement of the plunger 346 in the proximal direction draws fluid into the interior chamber 344 via the port 350, and wherein movement of the plunger 346 in the distal direction expels fluid from the interior chamber 344 via the port 350. As shown in FIG. 21, a portion of the plunger 346 protrudes from the enclosure 302 through a plunger opening 352. In some embodiments or aspects, the syringe drive of the delivery system may be configured to engage a proximal end of the plunger 346 that protrudes through the plunger opening 352. In other embodiments or aspects, the plunger 346 may be entirely contained within the housing 302 of the fluid cassette 300.

With reference to FIG. 24, the barrel 338 of the syringe 312 has a flange 354 protruding radially outward from the barrel 338 at the distal end 342. The flange 354 is configured to be received in a flange slot 356 on the enclosure 302 to prevent movement of the barrel 338 relative to the enclosure 302 as the plunger 346 is reciprocally moved within the barrel 338. The flange slot 356 may have a tapered geometry to ensure a tight fit with the flange 354.

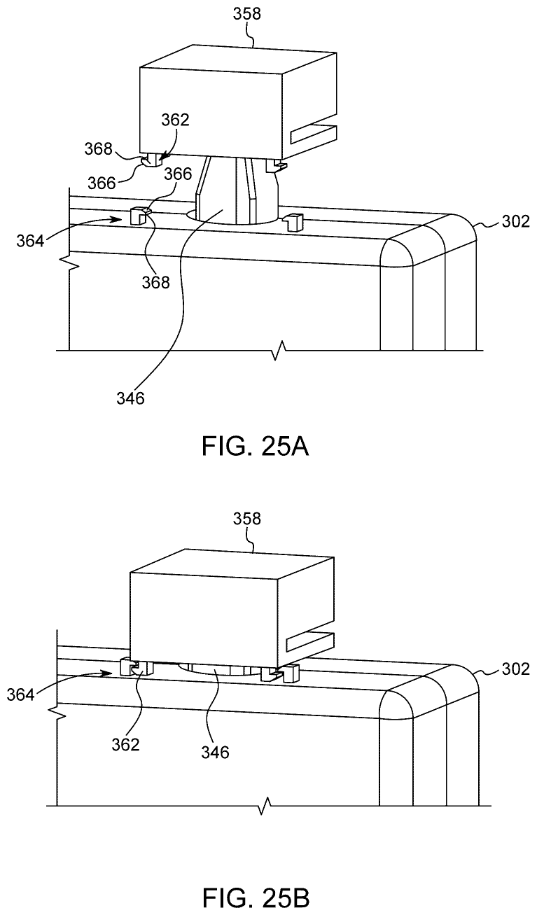

With reference to FIGS. 25A-25B, the plunger 346 has a plunger cap 358 configured for locking the plunger 346 in a locked position and preventing movement thereof such that filling and dispensing functionality of the syringe 312 (shown in FIG. 22) is disabled. In some embodiments or aspects, the plunger cap 358 may be shipped in a locked configuration and the injector 170 may be configured to unlock the plunger cap 358 to permit movement of the plunger 346 for filling and dispensing functionality. In some embodiments or aspects, the plunger cap 358 has at least one first hook 362 that is configured to engage with at least one second hook 364 on the enclosure 302. Each of the at least one first hook 362 and the at least one second hook 364 may have an angled contact surface 366 and a catch 368 that is configured to engage once the two contact surfaces 366 slide past each other. FIG. 25B shows the at least one first hook 362 and the at least one second hook 364 in a locked engagement with each other when the plunger cap 358 is urged toward the enclosure 302. Due to this locked engagement, the plunger 346 cannot be moved to fill the syringe 312 with fluid or dispense fluid from the syringe 312. The plunger cap 358 is further configured to maintain a position of the syringe 312 in a set position such that the plunger 346 can be connected to the plunger drive mechanism during installation of the fluid cassette 300 into the delivery device 100.

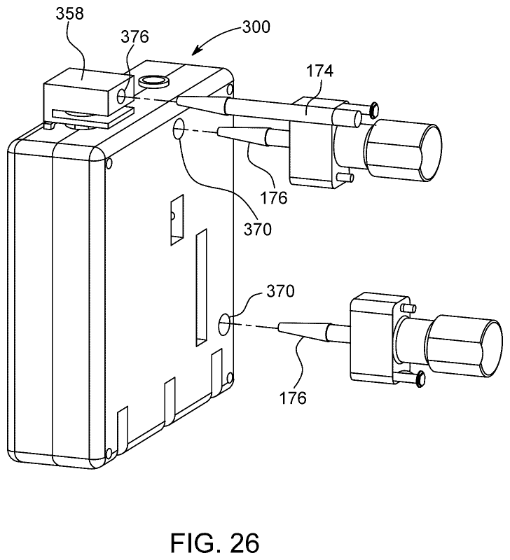

With reference to FIG. 26, the fluid cassette 300 has one or more alignment elements 370 for aligning the fluid cassette 300 relative to the injector 170 of the delivery system 100 (shown in FIG. 5). In some embodiments or aspects, the enclosure 302 of the fluid cassette 300 has a pair of alignment elements 370 configured as openings shaped to receive alignment pins 176 of the injector 170 (also shown in FIG. 31). Each of the pins 176 has a tapered surface configured for locating the alignment elements 370 on the fluid cassette 370 as the alignment pins 176 are moved in a direction toward the fluid cassette 370. Once the alignment pins 176 are inserted into the alignment elements 370, the fluid cassette 300 is positioned in a desired position relative to the injector 170 such that the spike 310 and the plunger 346 can be operated. For example, alignment of the alignment pins 176 with the alignment elements 370 on the fluid cassette 300 also aligns a delivery mechanism 174 with a corresponding plunger drive receiver 376 on the plunger cap 358 to move the plunger 346 during filling and dispensing operations. The plunger drive receiver 376 may have a tapered shape that corresponds to a tapered shape of the pin on the delivery mechanism 174. In some embodiments or aspects, the alignment elements 370 further facilitate alignment of valves and sensors of the delivery device 100 with corresponding locations on the fluid path set 314 in the fluid cassette 300.

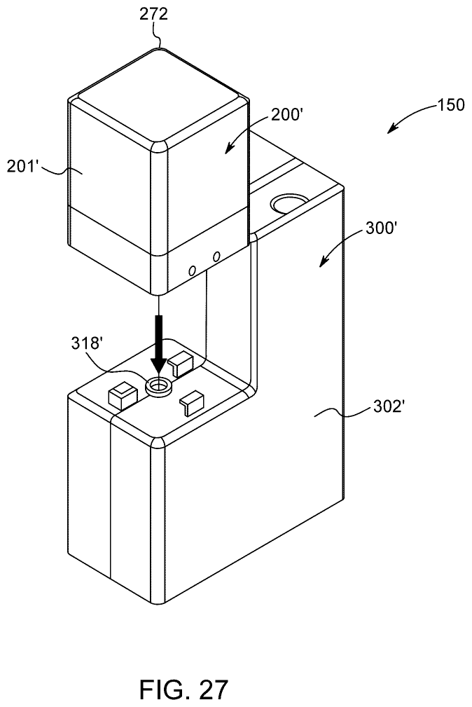

With reference to FIGS. 27-30, a fluid cassette 300′ is shown in accordance with another embodiment or aspect of the present disclosure. As the structure of the fluid cassette 300′ shown in FIGS. 27-30 is substantially similar to the structure of the fluid cassette 300 shown and described with reference to FIGS. 20-26, a detailed description of the components of the fluid cassette 300′ will be omitted. Same reference numbers will be used in FIGS. 27-30 to describe the components of the fluid cassette 300′ as used in FIGS. 20-26 to describe the components of the fluid cassette 300, except for the addition of a “′” mark after each reference number in FIGS. 27-30. The following detailed disclosure will focus only on relative differences between the two storage devices.

With reference to FIG. 27, the fluid cassette 300′ has an enclosure 302′ with a recess 378 shaped to receive the storage device 200′ shown in FIGS. 15-19. In some embodiments or aspects, the recess 378 is shaped such that the storage device 200′ is connected to the fluid cassette 300′, the resulting assembly 150 has a substantially cuboid shape. As discussed herein with reference to FIGS. 15-17B, the housing 201′ of the storage device 200′ includes a guide mechanism 272 that is configured for positioning the storage device 200′ in a desired orientation relative to the fluid cassette 300′. For example, with reference to FIG. 28, the guide mechanism 272 includes one or more geometric features such as grooves, chamfers, projections, holes or tabs, etc. that can be configured to mate with corresponding guide features 380 of the fluid cassette 300′ for providing a direct connection of the storage device 200′ to the fluid cassette 300′ in a pre-determined orientation. With reference to FIG. 29, the fluid cassette 300′ has one or more locking elements 382 for non-removably connecting the storage device 200′ to the fluid cassette 300′.

With reference to FIG. 31, an exemplary fluid diagram is shown illustrating fluid pathways between the storage device 200 and the fluid cassette 300. As shown in FIG. 31, the vessel 226 of the storage device 200 is fluidly connectable to the fluid path set 314 of the fluid cassette 300 via the spike 310. The fluid path set 314 may have a plurality of valves to control flow of fluid from the vessel 226 to an infusion set 406. In some embodiments or aspects, a first valve 384 is provided downstream of the spike 310 to regulate fluid flow into the fluid path set 314 from the spike 310. In some embodiments or aspects, the first valve 384 may be a pinch valve, a stopcock valve, or any other type of a valve configured for selectively permitting fluid flow from the spike 310 into the fluid path set 314. In some embodiments or aspects, the first valve 384 may be operable between an open position and a closed position by the injector 170.

The flow path of the fluid path set 314 can be structured to facilitate air removal during priming and limit the formation of bubbles within the material being passed from the vessel 226 of the storage device 200 to the syringe 312, for example by limiting abrupt changes or transitions in the internal diameter of the tubing of the fluid path set 314. The flow path of the fluid path set 314 can further be structured to prevent any air bubbles from being passed from the syringe 312 to incorporate a tortuous fluid path to preferentially separate and divert bubbles or with a hydrophobic membrane. The flow path of the fluid path set 314 can be designed to incorporate valves or other fluidic control elements such as passive valve, active valves, one-way valves, diverting valves, pinch valves, rotary valves, stopcocks, or on-off valves.

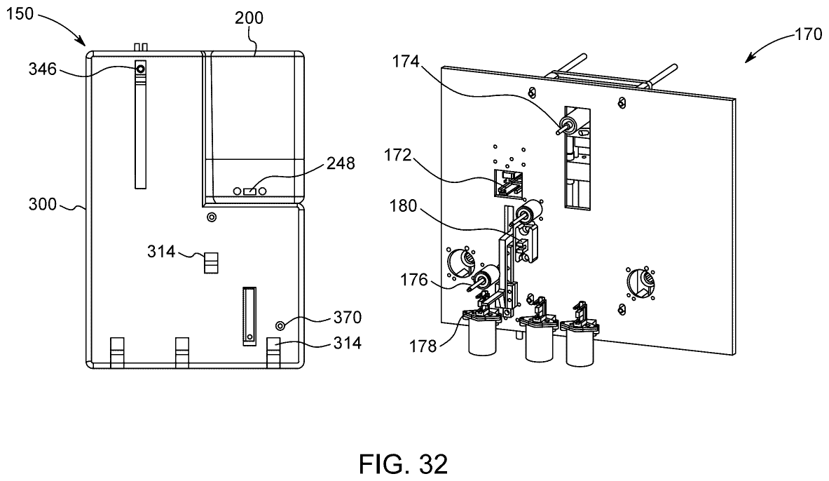

With continued reference to FIG. 31, an air detector 180 is provided downstream of the first valve 384. The air detector 180 is configured to detect air within the fluid path set 314. In some embodiments or aspects, the air detector 180 is provided on the injector 170 (see FIGS. 5 and 32) and the fluid cassette 300 is positioned relative to the injector 170 such that the air detector 180 is configured to detect air in the fluid path set 314 at a location downstream of the first valve 384. In some embodiments or aspects, output from the air detector 180 may be used by the controller 114 of the delivery system 100 (shown in FIG. 5) to permit or prevent operation of the injector 170 depending on the absence or the presence of air in the fluid path set 314. The air detector 180 may be configured to detect the presence of the therapeutic or diagnostic agent in the line from the vessel 226 to aid with volumetric accuracy of dose delivery.

With continued reference to FIG. 31, the air/particle filter 317 is provided downstream of the air detector 180. In some embodiments or aspects, a valve block 316 is provided downstream of the air detector 180. In some embodiments or aspects, the valve block 316 may be a manifold having a plurality of ports that can be selectively opened or closed to permit or restrict fluid flow therethrough. For example, the valve block 316 may have a first port 388, a second port 390, and a third port 392. The valve block 316 is operable such that only two of the three ports can be in fluid communication with each other. For example, if the valve block 316 is arranged such that the first and second ports 388, 390 are in fluid communication with each other and in fluid isolation from the third port 392, the syringe 312 can be filled with the therapeutic or diagnostic agent from the vessel 226 via the port 350. If the valve block 316 is arranged such that the second and third ports 390, 392 are in fluid communication with each other and in fluid isolation from the first port 388, fluid from an auxiliary fluid source 394, such as a saline source, can be delivered to the port 350 of the syringe 312 via an auxiliary line 396. In this configuration, saline or other fluid can be delivered for a patency check, a test infusion, or a flush procedure, as discussed herein. The auxiliary line 396 is connectable to an auxiliary branch 398 of the fluid path set 314. The auxiliary line 396 has a spike 395 for connecting to the auxiliary fluid source 394, a check valve 397, and a pair of connectors 399.

In some embodiments, a second valve 400 may be provided on the auxiliary branch 398 for controlling fluid flow to the valve block 316. In some embodiments or aspects, the valve block 316 can be operated to selectively open or close the first, second, and third ports 388, 390, 392 via the injector 170. Similarly, the second valve 400 may be operable between open and closed positions via the injector 170.

With continued reference to FIG. 31, a third valve 402 is provided downstream of the port 350 of the syringe 312. The third valve 402 may be operable between open and closed positions via the injector 170. An air/particulate filter 403 is provided downstream of the third valve 402 prior to the fluid path set 314 terminating in an end connector 404.

With continued reference to FIG. 31, the infusion set 406 is removably connectable to the fluid path set 314 of the fluid cassette 300 via the end connector 404. The infusion set 406 has a proximal connector 408 configured for removably connecting with the end connector 404 of the fluid path set 314. In some embodiments or aspects, the end connector 404 and the proximal connector 408 may be Luer connectors. The infusion set 406 further has a distal connector 410 configured for connecting to a catheter 412 or a priming cap 414. A pair of check valves 416 are provided between the proximal and distal connectors 408, 410. In some embodiments or aspects, the infusion set 406 may be configured for connecting to a sensor arrangement 416 having an occlusion detection sensor 418 and an air detector 420. In some embodiments or aspects, the occlusion detection sensor 418 may be configured for pressure testing the integrity of the fluid path set 314 prior to access to the vessel 226.

With reference to FIG. 32, the fluid cassette 300 and storage device 200 are shown along with components of the injector 170 configured for interacting with the fluid cassette 300 and the storage device 200. In some embodiments or aspects, the injector 170 includes an access mechanism 172 configured for moving the door 248 of the storage device 200 (shown in FIG. 8) from the closed position to the open position. The injector 170 further includes a delivery mechanism 174 configured for actuating the plunger 346 of the syringe 312 to fill the syringe 312 with the therapeutic or diagnostic agent from the storage device 200 or to fill the syringe 312 with saline from the auxiliary fluid source 394 (shown in FIG. 31). The delivery mechanism 174 may be further configured for actuating the plunger 346 of the syringe 312 to deliver the contents of the syringe 312, such as the therapeutic or diagnostic agent, or saline, to the infusion set 406 (shown in FIG. 31). While the fluid cassette 300 and fluid path elements thereof have been shown with a single syringe 312 and valving to do the fluid movement and control, pumps other than a single syringe 312 may be used. In some embodiments or aspects, there may be multiple pumps, for example one syringe 312 each for the drug and the flushing fluid. In some embodiments, one or more of the pumps may be a peristaltic pump, a diaphragm pump, or a piston pump. In some embodiments, the additional pump may eliminate the need for some valves or may benefit from the use of additional valves. In some embodiments it is desirable to have separate pumps for drug and flushing fluid from auxiliary fluid source 394 to provide the ability to have dual flow, that is delivering the two fluids simultaneously, so that total volumetric flow rate may be set independently of drug delivery rate. One benefit of dilute introduction is to possibly reduce chance of patient discomfort or reaction. A second is to reduce the time that the TRT is in the infusing vein before it is conducted to and diluted in the central circulation. See for example U.S. 2021/0187186 A1, which incorporated herein by reference.

With continued reference to FIG. 32, the fluid injector has one or more alignment pins 176 configured for engaging with the one or more alignment elements 370 on the fluid cassette 300. Once the alignment pins 176 are inserted into the alignment elements 370, the fluid cassette 300 is positioned in a desired position relative to the injector 170 such that the spike 310 and the plunger 346 can be operated. The injector 170 further has the air detector 180 configured for detecting air within the tubing of the fluid path set 314. In some embodiments or aspects, the injector 170 further may have a fluid detector configured for detecting the presence of a fluid and/or other properties relating to the fluid. In some embodiments or aspects, the injector 170 further has a valve assembly 178 configured for selectively engaging the tubing of the fluid path set 314 to regulate fluid flow therethrough.

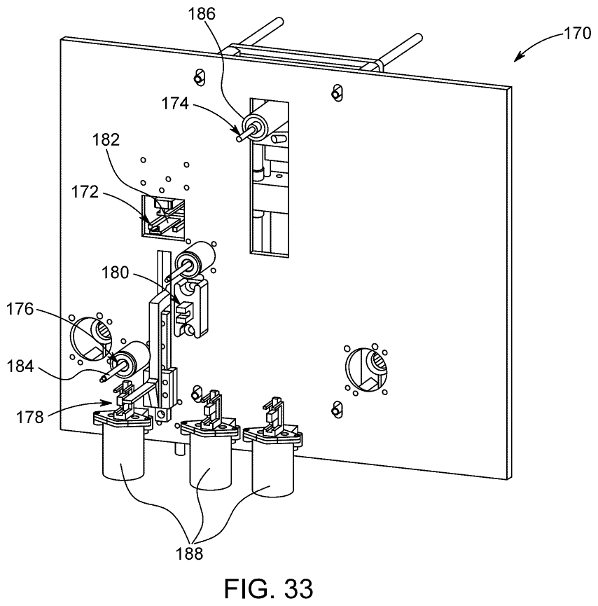

Once the storage device 200 is coupled to the fluid cassette 300, and the combined assembly 150 is installed in the delivery system 100, the access mechanism 172 of the injector 100 is configured to move the door 248 from the closed position to the open position such that the spike 310 of the fluid cassette 300 can be extended to pierce through the access port 232 of the vessel 226. With reference to FIG. 33, the access mechanism 172 of the injector 170 may have a probe 182 configured to extend in a direction toward the door 248 through the access opening 254 in the door cover 252 (shown in FIG. 9). In some embodiments or aspects, the probe 182 may be configured to pierce through the seal 256 on the access opening 254. The probe 182 may be configured to sense the presence of the seal 256, such as by sensing a resistance to movement through the door access opening 254 when the seal 256 is present compared to a resistance to movement when the seal 256 is absent. If the probe 182 does not detect the seal 256, such as due to no resistance to movement through the door access opening 254, the controller 114 (shown in FIG. 5) may be configured to prevent operation of the delivery system 100 because a used storage device 200 (i.e., one with a pierced seal 256), or a tampered storage device 200 (i.e., one with a removed seal 256) has been installed on the fluid. Operation of the probe 182 may be controlled via the controller 114.

With continued reference to FIG. 33, the access mechanism 172 of the fluid injector further may include a spike driver 184 configured for engaging with a spike drive slot 336 (shown in FIG. 23) of the spike 310. The spike driver 184 may be movable in a linear direction from a first position, which corresponds to a retracted state of the spike 310, and a second position, which corresponds to an extended state of the spike 310 in which the spike 310 pierces the access port 232 of the vessel 226. Operation of the spike driver 184 may be controlled via the controller 114.

With continued reference to FIG. 33, the delivery mechanism 174 includes a plunger driver 186 configured for actuating the plunger 346 of the syringe 312 to cause the plunger 346 to move within the barrel of the syringe 312. The plunger driver 186 is shaped to be received in the plunger drive receiver 376 such that movement of the plunger driver 186 causes a corresponding movement of the plunger 346. The plunger driver 186 may have a motor for moving the plunger 346 in a linear direction. The plunger driver 186 may be movable in a linear direction in a first direction, in which the barrel of the syringe 312 is configured to be filled with fluid, and a second direction opposite the first direction, in which fluid from the barrel of the syringe 312 is configured to be delivered via the port 350. Operation of the plunger driver 186 may be controlled via the controller 114. In some embodiments or aspects, the plunger driver 186 determines a flow rate of the fluid that is delivered to the patient.

With continued reference to FIG. 33, the air detector 180 is configured for detecting air within the tubing of the fluid path set 314. The air detector 180 may be an optical air detector, an acoustic air detector, an ultrasonic air detector, or any other air detector configured for detecting a presence of air in the tubing of the fluid path set 314. Operation of the air detector 180 may be controlled via the controller 114.

With continued reference to FIG. 33, the valve assembly 178 may include a plurality of valves 188. In some embodiments or aspects, the plurality of valves 188 may be pinch valves configured to pinch the tubing of the fluid path set 314. In some embodiments or aspects, the valves 188 may be rotary stopcocks or other fluid flow shutoff mechanisms. Operation of the valve assembly 178 may be controlled via the controller 114.

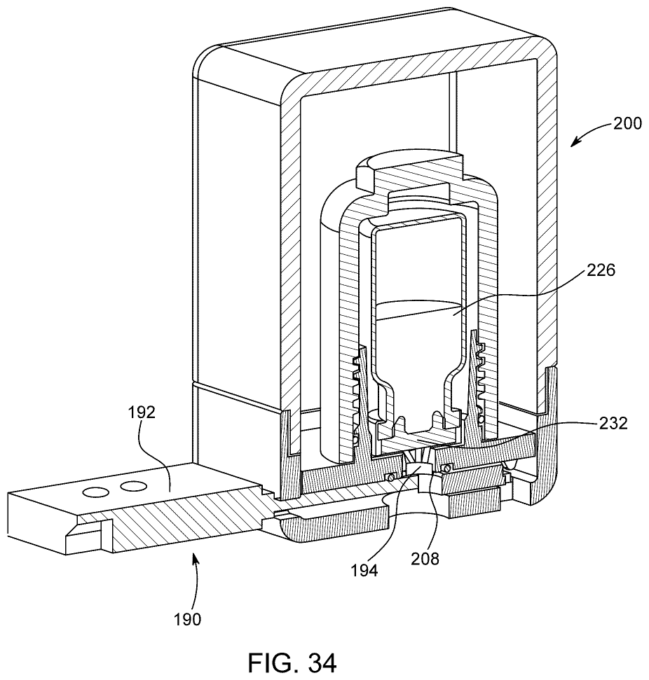

With reference to FIG. 34, a disinfection mechanism 190 is provided for disinfecting the access port 232 of the vessel 226. In some embodiments or aspects, the disinfection mechanism 190 includes a movable arm 192 and a disinfection source 194. The movable arm 192 is movable relative to the storage device 200 such that the disinfection source 194 may be positioned opposite the access port 232. The disinfection source 194 can include, for example, a laser or light emitter that can emit electromagnetic energy at wavelengths capable of inactivating organisms on the surface of the access port 232. Examples of such electromagnetic energy that can be emitted include ultraviolet light (UV) light (10-400 nanometer (nm) wavelength light), ultra-violet C light (UV-C) light (light having a wavelength of 200-280 nm), white light, infrared (IR) light, a laser, etc. (e.g. the disinfection mechanism can include a UV light emitter, UVC LED, IR emitter, etc.). The emitted light can be emitted continuously onto the surface of the access port 232 for a pre-selected disinfection time period to deliver a sufficient energy dose to inactivate organisms on the access port 232 prior to the spike 310 being inserted through the access port 232 and into the vessel 226. In some embodiments or aspects, the disinfection source 194 may be configured to disinfect the access port 232 of the vessel 226 and the spike 310. In this manner, the access port 232 and the spike 310 are disinfected for a sterile connection therebetween. In further embodiments or aspects, a second disinfection source 194 may be provided on the movable arm 192 for disinfecting the spike 310 prior to insertion of the spike 310 into the access port 232 of the vessel 226.

In some embodiments or aspects, the disinfection source 194 can include a nozzle or sprayer that can spray an antiseptic material onto the access port and/or an agitation mechanism that can wipe an antiseptic agent onto the access port of a pre-selected sanitation time period. The disinfection time period that is selected can be based on the type of antiseptic that is utilized and the time period needed to eliminate a pre-selected set of organisms with that antiseptic or reduce the amount of such organisms to at or below a pre-selected threshold level. In some embodiments or aspects, the antiseptic material may be applied at the manufacturing site via an antiseptic containing absorbent member similar to that in a SwabCap made by ICU Medical, Inc. of San Clemente, California. The door 248 may hold the antiseptic containing absorbant member in contact with the access port. The antiseptic, for example 70% isopropyl alcohol disinfects, then evaporates slowly. The continued presence of the absorbent member held by the door 248 maintains the sterility of the access port. The absorbent member is moved with door 248 to allow access to the access port.

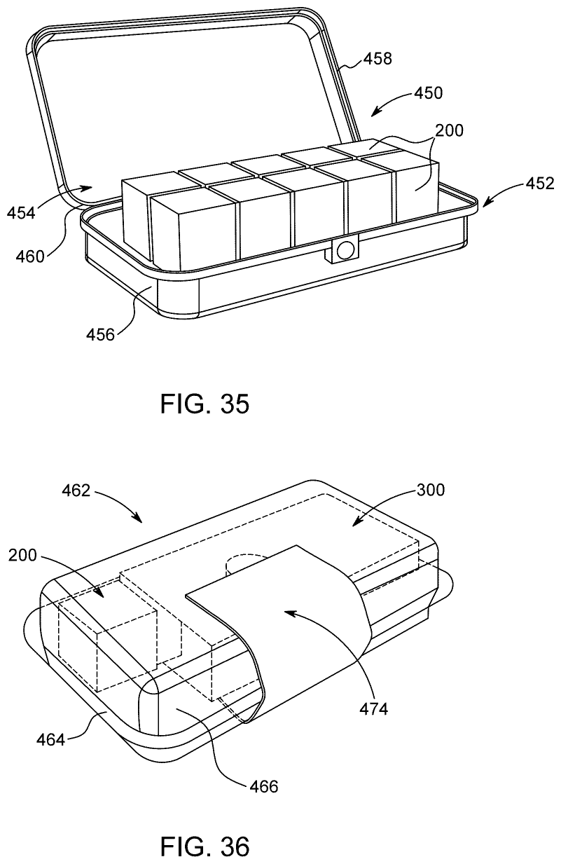

With reference to FIG. 35, a storage container 450 for containing a plurality of storage devices 200 is shown in accordance with one embodiment or aspect. In some embodiments or aspects, the storage container 450 may be configured to contain the storage devices 200 during shipping and storage prior to use. The storage container 450 has a housing 452 defining an interior 454 that is configured to receive a plurality of storage devices 200 therein. The housing 452 may have a receiving portion 456 and a lid portion 458 connected to the receiving portion 456 by a hinge 460. In some embodiments or aspects, the housing 454 of the storage container 450, such as at least one of the receiving portion 456 and the lid portion 458, may provide additional shielding properties to provide increased radiation shielding capabilities. In this manner, radiation emitted by the therapeutic or diagnostic agent contained within the storage devices 200 can be contained during shipment and storage.

After the therapeutic or diagnostic agent is output from the storage device 200 and injected into a patient, materials used to inject the therapeutic or diagnostic agent into the patient are collected for storage and disposal. With reference to FIG. 36, a disposal container 462 is provided for containing such materials. In some embodiments or aspects, the disposal container 462 is configured to contain the storage container 200, the fluid cassette 300, and infusion tubing 406 used during an injection procedure. The disposal container 462 has a housing 464 defining an interior 466 that is configured to receive the used storage container 200, fluid cassette 300, and infusion tubing 406. In some embodiments or aspects, the housing 466 of the disposal container 462, may provide shielding properties to provide radiation shielding capabilities. In this manner, radiation emitted by the used storage container 200, fluid cassette 300, and infusion tubing 406 can be contained for safe disposal. In some embodiments or aspects, the disposal container 462 may be configured to seal any remaining fluid in the used storage container 200, fluid cassette 300, and infusion tubing 406.

With continued reference to FIG. 36, a label 474, optionally printed by the delivery system 100, can be applied onto the disposal container 462 to prevent the disposal container 462 from being opened after the used materials are placed into the interior thereof. The label 474 may also provide information about when the materials were used and when the disposal container 462 has been stored long enough for subsequent disposal. The label 474 can have a bar code or RFID tag so that disposal information can be provided to a computer in response to a bar code scanner or RFID reader reading the label 474.

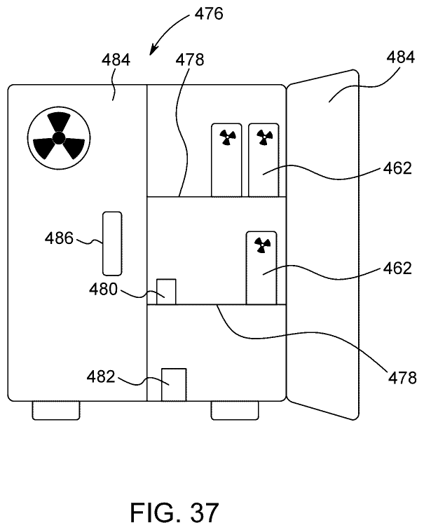

Once the used materials have been placed within a labeled disposal container 462, the disposal container 462 can be temporarily stored in the cart of the delivery system 100. In some embodiments or aspects, the labeled disposal container 462 may be stored in a disposal locker 476, shown in FIG. 37. In some embodiments or aspects, the disposal locker 476 may have a plurality of drawers or shelves 478 each configured to retain a plurality of disposal containers 462. The label 472 on the disposal container 462 can be scanned by a user having a mobile bar code reader. If the read bar code indicates that the materials within the disposal container 462 are sufficiently decayed that they are safe for disposal, an alert, such as a message, sound and/or color can indicate that the scanned disposal container 462 can be removed from the disposal locker 476 and disposed of using an approved disposal process.

In some embodiments or aspects, drawers or shelves 478 of the disposal locker 476 can have at least one indicator 480 (e.g. red and green LEDs) configured to indicate whether a particular disposal container 462 on the drawer or shelf 478 is safe for disposal. For example, a user can scan a bar code of an individual disposal container 462 and provide other input to an inventory management computer 482 to indicate that the individual disposal container 462 has been added to the drawer or shelf 478. The inventory management computer 482 can then determine whether the stored materials within each specific disposal container 462 have decayed sufficiently based on the scanned information related to the used materials (e.g. date of use, etc.) to control the state of the at least one indicator 480. For example, the inventory management computer 482 can control the state of the at least one indicator 480 such that an LED or other indicator means of the at least one indicator 480 indicates the material in the disposal container 462 is too radioactive to throw away (such as by displaying a red color or other message) or such that an LED or other indicator means of the at least one indicator 480 indicates the material in the disposal container 462 can be thrown away (such as by displaying a green color or other message). Such indicia can permit a user to quickly determine whether the disposal container 462 can be thrown away. This can avoid a user having to periodically scan containers or check use dates on the label 474 of each disposal container 462 to determine the disposal status of the disposal container 462.

The disposal locker 476 can have doors 484 to enclose an interior thereof and a locking mechanism 486 for locking the doors 484. In some embodiments or aspects, the locking mechanism 486 may be configured such that only a user with a sufficient credentials can open the doors 484 to access the disposal locker 476. For example, the locking mechanism 486 may be such that the user must have a key to unlock the doors 484 or must have a user badge or access associated with a user log-in to provide input to a controller for unlocking the doors 484.

Among the functions, capabilities and benefits provided by the systems and methods described herein is the minimization of connections that must be made, the minimization of connections that must be separated or broken, and the containment as much as possible of each connection. In some embodiments, methods, or uses of the system, the only connection that is separated is the connection to the patient, and this is preferable only done once all the drug has been delivered and the delivery connection flushed of drug. Thus, there is a much-reduced chance of any drips, spills, or leakage of liquid drug, aerosols, vapors, or gasses being released which may pose a danger to operators or others in the vicinity. Some radioactive daughter products are gasses. Chemotherapeutic aerosols can be a hazard to those in the vicinity.

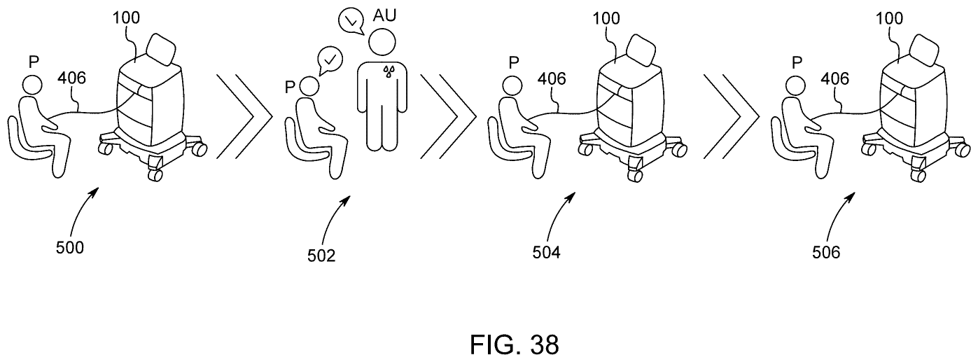

With reference to FIG. 38, an exemplary process for a test infusion prior to delivery of the therapeutic or diagnostic agent is shown. At 500, the patient P is connected to the delivery system 100 via the infusion set 406 and the patient P is administered a test injection of saline or other fluid. For example, the injector 170 can be operated to fill the syringe 312 with saline or other fluid from the auxiliary fluid source 394 and deliver a test injection of saline or other fluid to the patient via the infusion set 406. Volume of saline or other fluid delivered to the fluid can be sufficient to confirm whether saline or other fluid is delivered to the patient's vasculature or is extravasated or leaks into tissue. At 502, the patient P and the authorized user AU administering treatment to the patient P confer whether the test injection of saline or other fluid was successful. For example, the authorized user AU may visually check the injection site for signs of extravasation and/or palpate the injection site. The patient P can report any discomfort associated with the test injection of saline or other fluid.

With continued reference to FIG. 38, at p 504, the patient P is administered a pre-infusion of saline or other fluid. For example, the injector 170 can be operated to fill the syringe 312 with saline or other fluid from the auxiliary fluid source 394 and deliver the test infusion of saline or other fluid to the patient via the infusion set 406 at a higher volume than during the test injection at 500 and for a longer duration than during the test injection at 500. In some embodiments or aspects, the infusion volume, infusion duration, and/or infusion rate are selected to correspond to the infusion volume, infusion duration, and/or infusion rate for delivery of the therapeutic or diagnostic agent. Research as demonstrated that starting infusions with saline at the full infusion rate reduces the likelihood of extravasation and gives time for an extravasation to be sensed if one is going to happen. At 506, the patient P is administered an injection of the therapeutic or diagnostic agent using a pre-determined injection protocol. This process may be continuous with the pre-infusion of the previous procedure unless the operator intervenes.

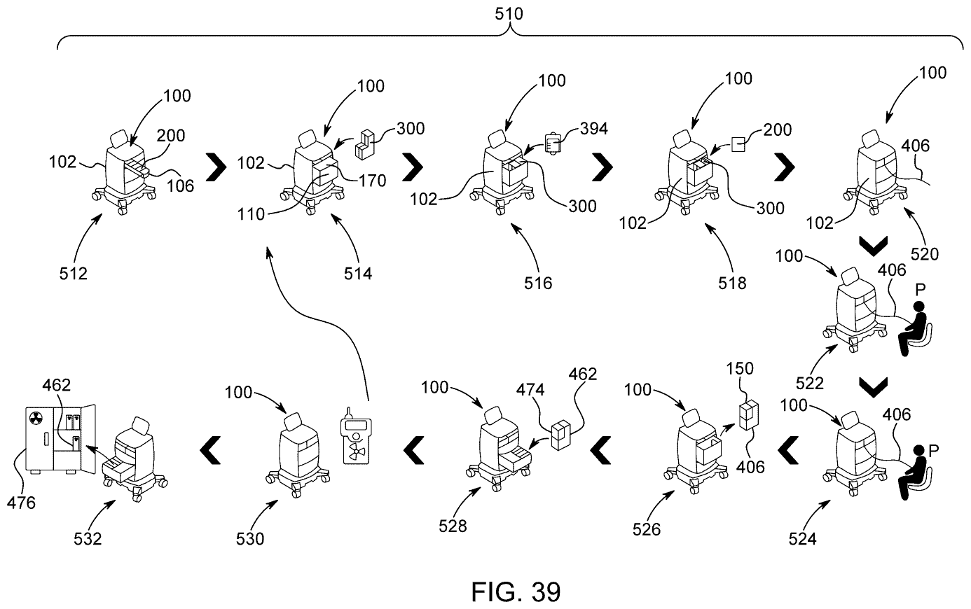

With reference to FIG. 39, an exemplary process for administering a therapeutic or diagnostic agent using the delivery system 100 is shown. At 510, the delivery system 100 is prepared for an administration procedure. For example, at 512, one or more storage devices 200 are loaded into the one or more storage compartments 106 of the cart 102. At 514, a fluid cassette 300 is loaded into the injector 170. For example, the fluid cassette 300 is positioned within the second drawer or shelf 110 of the cart 102 such that the fluid cassette 300 engages the injector 170. In some embodiments or aspects, the alignment elements 370 on the fluid cassette 300 are configured to engage with the alignment pins 176 of the fluid injector (shown in FIG. 26) to locate the fluid cassette 300 relative to the injector 170 such that various components of the injector 170 can interface with the corresponding components of the fluid cassette 300.

With continued reference to FIG. 39, at 516, an auxiliary fluid source 394 is connected to the fluid cassette 300. For example, the auxiliary fluid source 394 may be spiked and fluidly connected to the fluid path set 314 of the fluid cassette 300 via the auxiliary line 396 (shown in FIG. 31). At 518, a storage device 200 is connected to the fluid cassette 300. In some embodiments or aspects, the label, tag, or other indicia 270 on the storage device 200 (shown in FIG. 16) may be scanned prior to or at the time of connecting with the fluid cassette 300 in order to load information about the contents of the storage device 200 into the controller 114.

At 520, the infusion set 406 is fluidly connected to the fluid path set 314 of the fluid cassette 300 (shown in FIG. 31), and the fluid path set 314 and the infusion set 406 are primed. In some embodiments or aspects, the controller 114 of the delivery system 100 (shown in FIG. 5) may be configured to initiate a priming procedure wherein the syringe 312 is operated to draw fluid from the auxiliary fluid source 394 into the fluid path set 314 and deliver the fluid into the infusion set 406 to prime the fluid path set 314 and the infusion set 406 with fluid.

With continued reference to FIG. 39, a test injection procedure is performed at 522. In some embodiments or aspects, the test injection procedure may include 500-504 described herein with reference to FIG. 38.

At 524, the delivery system 100 is configured to administer the therapeutic or diagnostic agent to the patient. For example, the syringe 312 may be operated to be filled with the therapeutic or diagnostic agent from the vessel 226 of the storage device 200, and to deliver the therapeutic or diagnostic agent to the patient P via the infusion set 406 based on a pre-determined administration protocol. In some embodiments or aspects, the delivery system 100 can be configured to administer a unit dose to the patient, wherein a unit dose requires a delivery of the entire contents of the vessel 226. In other embodiments or aspects, the delivery system 100 can be configured to administer a non-unit dose to the patient, wherein a non-unit dose requires a delivery of a portion of the entire contents of the vessel 226.

With continued reference to FIG. 39, at 526, upon completion of the administration procedure, the infusion set 406 is disconnected from the patient P and the assembly 150 of the storage device 200 and the fluid cassette 300 is removed from the injector 170. At 528, the used storage device 200, fluid cassette 300, and infusion set 406 are placed in the disposal container 462, and a label 474 is applied to the disposal container 474 before placing it in temporary storage on the cart 102. For example, the disposal container 462 can be loaded into the third drawer or shelf 112 of the cart 102.

At 530, the treatment room is cleaned and the delivery system 100 can be readied for another administration procedure. At 532, the disposal container 462 is moved to the disposal locker 476 for further decay-in-place of the radioactive material.

The use of label, tag, or other indicia 270 on storage device 200 for administration of new doses and storage of used material for disposal can also provide sufficient information to prompt the ordering of new doses. For example, an inventory system can include a computer that receives information on the storage devices 200 that have been used, such as based on information contained on the label, tag, or other indicia 270 (shown in FIG. 16), and determine whether the number of available doses, such as based on the number of available storage devices 200, is below a pre-selected threshold for triggering the ordering of additional doses. Optionally, the inventory system may compare the number of available doses to the patient scheduling load. In some situations, the inventory system can communicate this condition to a distribution system so that an automated message is sent to the customer to help flag the low inventory and prompt the submission of a new purchase order for additional doses. The inventory system can include software configured to facilitate use of the inventory system. For example, the software can be configured to facilitate inventory management and ordering, written directive generation/acceptance, and compliance report generation. In addition, the use of label, tag, or other indicia 270 on storage device 200 for administration of new doses and storage of used material for disposal can also provide sufficient information to prompt the billing for the use of the drug and system if that is the business arrangement.

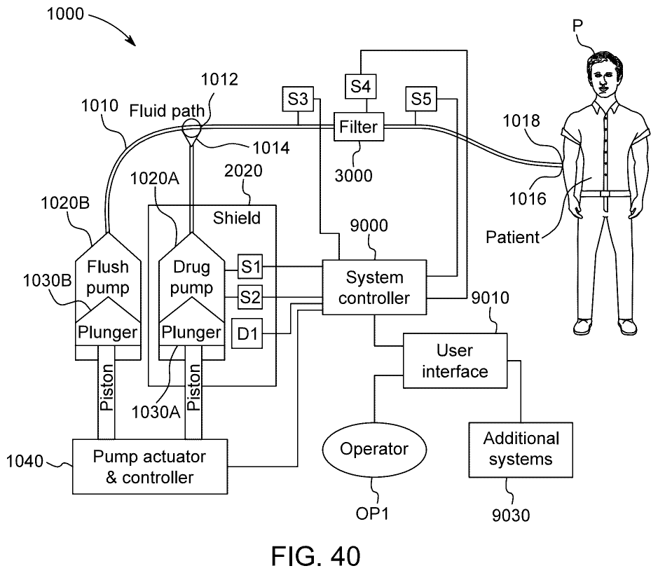

Referring now to FIG. 40, a schematic of a fluid injector system 1000 according to an embodiment of the present disclosure is illustrated. The system 1000 includes two fluid reservoirs 1020A, 1020B, which may be syringes as illustrated in FIG. 40. A first of the fluid reservoirs 1020A contains a therapeutic or diagnostic agent, such as a radiopharmaceutical drug, and a second of the fluid reservoirs 1020B contains a flushing agent, for example saline. Fluid may be injected from the fluid reservoirs 1020A, 1020B by respective plungers 1030A, 1030B, which are controlled by a piston actuator 1040 operated by a system controller 9000. The system controller 9000 may control essentially all of the automated functions of the system 1000, and may further include or be connected to a user interface for receiving inputs from the operator. The system controller 9000 may include at least one processor for executing various functions of the system 1000 as described herein. The system controller 9000 may further include a non-transitory computer-readable medium for storing instructions that, when executed by the at least one processor, cause the at least one processor to execute various functions of the system 1000 as described herein. The system controller 9000 includes or interfaces with a user interface 9010. An onsite operator OP1 interacts with the system through the user interface 9010. The onsite operator may be an authorized user AU. The system may also communicate with one or more additional or auxiliary systems 9030 which may include, for example image systems, hospital information systems, electronic medical record systems, picture archiving systems, radiology information systems, nuclear medicine information systems, cloud storage, cloud computing, and remove control systems. As discussed, there are legal, regulatory, and procedural requirements relating to who is licensed to do what aspects of the drug preparation and delivery. An additional system that may be accessed is a local, regional, or national data base that may be used to confirm licensure or qualifications to perform the process or function that is to be performed. The communications interface to one or more additional systems 9030 may be through the user interface as shown or directly from the system controller (not shown). The communications can utilize wired or wireless communications known to those skilled in the art provided the communications can be fast enough to allow effective interaction, operation, and where applicable control of the system.

In addition to data, information, and communication system, additional systems 9030 may be medical patient measurement or monitoring systems, for example ECG, pulse oximeter, temperature, motion, hydration state, cardiac output, and/or respiration rate. In some embodiments or aspects, an additional system 9030 may, for example, be a camera on a separate device that observes or monitors a patient. Data about the patient may be used to monitor the patient's state, check for distress, and/or detect the development of an adverse event during the infusion, as discussed in WO 2021/222771 A1, which is fully incorporated herein by reference. Additional systems 9030 may also be a medical measurement system, such as a CT imager, MR imager, ultrasound unit, or similar device which makes measurements of a patient. Additional system 9030 may be a medical output or treatment device which performs a function on or does an action on or to the patient. Such a device may, for example, be one or more infusion pumps that infuses flushing fluids, protectants, and/or other drugs into a different or the same IV in the patient. The infusion pump may infuse additional saline with the goal of establishing a significantly flow into and through the patient's veins to reduce the chance of retention in the vein. A significant flow in adults, whether provided by an additional fusion pump or by the pump(s) of this disclosure is at least 0.1 ml/s, preferable 0.5 ml/s and ideally 1.0 ml/s. The flow rate that may be use is limited by the total desired time of the infusion and the need to not volume overload the patient. In some instances, because of the volume overload risk, it may be desirable to divide the dosing into multiple discrete boluses with a pause or a reduction of saline flow between boluses. Each bolus is a bolus of drug followed by a flush bolus that flushes the drug out of the neighborhood of the venous injection site. In some embodiments it is desirable that the additional system 9030 communicate its status with the system controller 9000. It is further desirable that the controller 9000 be able to activate, program, or control the additional system 9030 so that the overall infusion can be well coordinated. In an embodiment where an additional system 9030 is an infusion pump, that infusion pump may be used in the patency check procedure of FIG. 38.

The fluid reservoirs 1020A, 1020B are fluidly connected to a patient P via a fluid path set 1010 comprising various fluid path elements. The fluid path set 1010 includes a T-connection 1012 where outlet lines from the fluid reservoirs 1020A, 1020B merge into a single line for delivery to the patient P. A valve 1014 is incorporated into the T-connection 1102 or located upstream of the T-connection 1012 to prevent radiopharmaceutical from flowing through the T-connection 1012 during priming of the system 1000. Suitable embodiments of the valve 1014 include, for example, a check valve, a high crack pressure valve, or a stopcock. An example of a commercially available component including the T-connection 1012 and valve 1014 is an SSIT-96LV sold by Bayer, AG. Various components of the system 1000 may be configured for single-patient use and/or multi-patient use. For example, portions of the fluid path set 1010 and may be configured for single-patient use, and may be disposed of after each use. The fluid reservoirs 1020A, 1020B may be configured for single-patient use or multi-patient use in various embodiments. Further details of single- and multi-patient configurations of various components in the system 1000 are described in U.S. Pat. Nos. 8,926,569, 9,855,390, 9,173,995, 9,700,670, 9,480,797, 10,039,889, 10,512,721, and 10,668,221, the disclosures of all of which are hereby incorporated by reference in their entireties.

With continued reference to FIG. 40, the first fluid reservoir 1020A may be at least partially encased by a shield 2020 to protect operators and patients from unnecessary radiation exposure. The structure and material of the shield 2020 may be selected based on the radioactive properties of the radiopharmaceutical contained in the first fluid reservoir 1020A, such as the type and the amount of radiation emanating from the radiopharmaceutical and/or the quantity of the radiopharmaceutical. Exemplary types of radiation emitted are gamma radiation, beta radiation, alpha radiation, and positron. These types differ, for example in their penetration of shielding and damage to tissue. The amount of radiation from a source is generally measured in decays per second or Becquerel in the SI system of units. In instances where the radiopharmaceutical is expected to emanate relatively high amounts of highly penetrating radiation, the shield 2020 may be composed of a thick layer of lead or tungsten. In instances where the radiopharmaceutical is expected to emanate relatively low amounts of weakly penetrating radiation, the shield 2020 may be composed of a thin layer of plastic or be distance only. In other embodiments of the system 1000, the first fluid reservoir 1020A containing the radiopharmaceutical is located at a predetermined safe distance away from the operator(s) and patient(s), such as in a separate room, to eliminate unnecessary radiation exposure.

With continued reference to FIG. 40, some embodiments of the system 1000 include one or more radiation measuring sensors S1, S2 associated with the first fluid reservoir 1020A. The sensors S1, S2 are configured to generate and transmit a signal (i.e. a voltage or digital signal) to the system controller 9000 related to the radiation emanating from the first fluid reservoir 1020A and impinging on them. In some embodiments, a one or more labels, tags, or data elements, D1 associated with the first fluid reservoir 1020A contains information about the contents of the first fluid reservoir 1020A, for example product information, production information, prescription information, shipping conditions information, drug type, total dose, time at which the radiopharmaceutical was prepared, and time at which the dose was measured. The data element D1 may also contain information about the position of the plunger 1030A within the first fluid reservoir 1020A, and other pertinent information relating to an injection procedure. The data element D1 interacts with the system controller 9000 to provide information used in subsequent computations and/or communications to the operator, data systems, or imaging systems. The data element D1 may be any type of data containing device, for example a printed label, a bar code, or an electronically readable device, for example a radiofrequency identification (RFID) tag. The data element D1 may itself contain the information (e.g. the drug type, total dose, time at which the radiopharmaceutical was prepared, time at which the dose was measured, plunger position, etc.), or the data element D1 may contain information such as an index or website address that directs the system controller 9000 to a location where the information can be retrieved, for example a secure, central database. The data element D1 may be writeable by the system controller 9000 to incorporate information during or after one or more injection procedures are performed using the radiopharmaceutical contained in the first fluid reservoir 1020A. For example, the system controller 9000 may update the data element D1 (or the central database indexed by the data element D1) with the remaining volume of radiopharmaceutical, position of the plunger 1030A, or number of patients injected. Some or all of the data elements D1, sensors S1, S2, and shield may be associated with the fluid reservoir 1020A and be transported from a radiopharmacy with the drug for subsequent incorporation into system 1000. Alternatively, some or all of the data elements D1, sensors S1, S2, and shield may be associated with system 1000 independent of the fluid reservoir that is considered permanent or semipermanent parts of system 1000.

With continued reference to FIG. 40, the fluid path set 1010 includes of one or more fluid path elements, e.g. tubing and valves, which carry the fluid from the fluid reservoirs 1020A, 1020B to the patient P. One of the fluid path elements of the fluid path set 1010 may include a filter 3000 for selectively retaining radioactive particles from the radiopharmaceutical passing though the filter 3000 en route to the patient P. A particle may, for example be an atom, molecule, chelate, agglomeration, colloid, or other physical structure which contains at least one radioactive atom. The filter 3000 is configured to selectively remove undesirable atoms or molecules, and/or particles in the radiopharmaceutical, while having no, minimal, or a controlled effect of the clinical properties of the radiopharmaceutical exiting the filter. For example, the filter 3000 may be configured to remove radium from a conjugated thorium radiopharmaceutical. The presence of radium in conjugated thorium is described in U.S. Patent Application Publication No. 2019/0002363, the disclosure of which is hereby incorporated by reference in its entirety. In another example, the filter 3000 may be configured to capture and remove generator isotopes in the radiopharmaceutical. In such example, the radiopharmaceutical may be supplied to the first fluid reservoir 1020A—or the first fluid reservoir 1020A may include—a generator which produces the radiopharmaceutical, such as a rubidium or technetium generator. Such a generator is described in U.S. Pat. No. 8,071,959, the disclosure of which is hereby incorporated by reference in its entirety. Filters similar to this may be configured to immobilize generator isotopes in the rubidium, gallium, or technetium generator. The filter 3000 may be at least partially constructed from materials similar or identical to those used for isotope immobilization in rubidium, gallium, or technetium generators. Thus, some amount of any breakthrough of isotopes from the generator will be trapped by the filter 3000.

With continued reference to FIG. 40, the system 1000 may include one or more sensors S3, S4, S5 associated with the fluid path set 1010 and in communication with the system controller 9000. For example, a filter radiation sensor S4 may be located in proximity to the filter 3000 to detect radiation accumulation in or on a portion of the filter material and/or in the fluid paths within filter 3000. Fluid path radiation sensors S3 and S5 may be located in association with the fluid path set 1010 upstream and downstream, respectively, of the filter 3000. The fluid path radiation sensors S3, S5 may thus measure radiation in the fluid path set 1010 on either side of the filter 3000. The system controller 9000 may utilize measurements from the sensors S3, S4, S5 to monitor radiation buildup and delivery throughout the system 1000, to assess efficiency and sufficiency of filtering, and to adjust or halt an injection procedure if radiation levels increase to a degree that threatens patient or operator health, or otherwise deviates from an expected level of radiation, for example is too much radioactivity being retained in filter 3000.

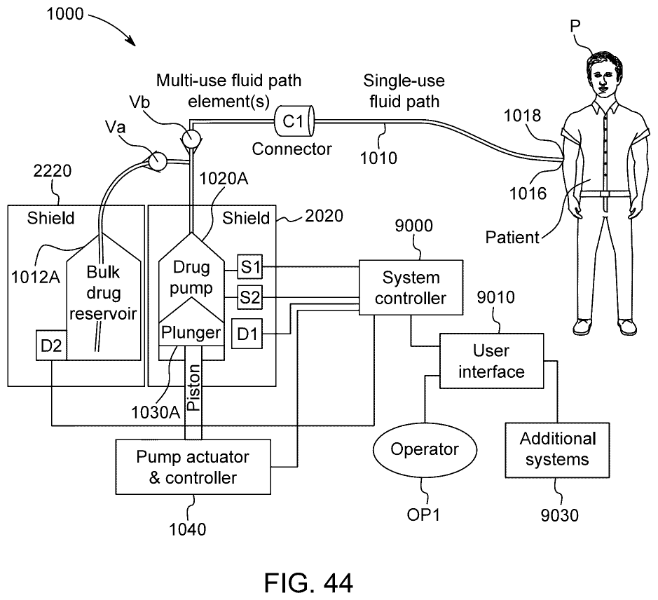

To prepare the system 1000 for performing an injection procedure, the elements of the fluid path set 1010 are assembled in the general configuration shown in FIG. 40 and placed in operative association with the sensors S1-S5. In some embodiments, the fluid reservoirs 1020A, 1020B are supplied prefilled with the respective fluids and connected to the fluid path set 1010. In some embodiments the shield 2020 and optionally sensors S1, S3, and device D1 are supplied in association with the prefilled syringe 1020A. In other embodiments, one or both of the fluid reservoirs 1020A, 1020B may be filled manually or automatically using additional components of the system 1000 (see, e.g., FIG. 44) prior to performance of an injection procedure. In some embodiments, some or all of the fluid path elements may come preconnected to simplify the use and reduce the chance of error in assembly.

Once the system is assembled for use, operation is as follows. The assembled fluid path set 1010 is primed before connecting to the patient by pumping fluid from the fluid reservoirs 1020A, 1020B into the fluid path set 1010 to displace any air. Priming may be performed by advancing the plunger 1030A associated with the first fluid reservoir 1020A such that the portion of the fluid path set 1010 from the first fluid reservoir 1020A to the T-connection 1012 is filled with the radiopharmaceutical. The valve 1014, preferably allows air to leave the fluid path element between first fluid reservoir 1020A to the T-connection 1012 and then is closed so that the radiopharmaceutical does not enter the portion of the fluid path set 1010 downstream of the T-connection 1012. After the portion of the fluid path set 1010 from the first fluid reservoir 1020A to the T-connection 1012 has been primed with radiopharmaceutical, the first plunger 1030A is halted and the second plunger 1030B associated with the second fluid reservoir 1020B is advanced so that the remainder of the fluid path set 1010 is filled with the flushing agent, e.g. saline, from the second fluid reservoir 1020B. The second plunger 1030B is then halted and the fluid path set 1010 is fully primed.

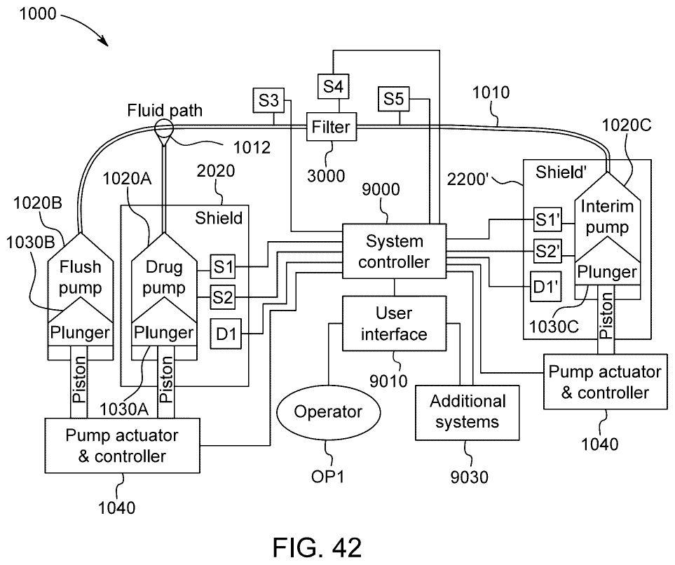

Once primed, the fluid path set 1010 may be connected to the patient P, for example via a catheter 1016 of the fluid path set 1010 inserted into a venous access site 1018 of the patient P. In other embodiments, the fluid path set 1010 may be indirectly connected to the venous access site 1018 of the patient P via an interim pump, as will be described herein with reference to FIGS. 42, 43, and 54. Referring still to the embodiment shown in FIG. 40, once the fluid path set 1010 is primed and connected to the patient P, the system controller 9000 may automatically, or through manual input of the operator, actuate the piston actuator 1040 to cause delivery of the radiopharmaceutical and, if prescribed in the injection procedure, the flushing agent.

The system controller 9000 may determine the volume of radiopharmaceutical to deliver to the patient P based upon an initial dose and volume or concentration of the drug in the first fluid reservoir 1020A, which is then corrected to account for any radioactive decay experienced by the radiopharmaceutical. The dose and concentration of the radiopharmaceutical may be known or derived from data stored in memory of the system controller 9000, or the system controller 9000 may determine the dose or concentration from radiation measurements taken by the sensors S1, S2 associated with the first fluid reservoir 1020A. The system controller 9000 may determine the volume of radiopharmaceutical in the first fluid reservoir 1020A based upon the position of the first plunger 1030A, which may be obtained from the data element D1 as described herein or from a sensor (e.g. and encoder) associated with the piston actuator 1040 and/or the first plunger 1030A. Additional description of calculating the initial dose and volume or concentration of the radiopharmaceutical are described in U.S. Pat. No. 10,016,618, the disclosure of which is hereby incorporated by reference in its entirety.

In some embodiments, the flushing agent may serve additional functions beyond priming of the fluid path set 1010. The flushing agent may also be injected before the radiopharmaceutical as a test injection to ensure system components are connected as intended and/or that there is good flow form the catheter 1016 into the patient's veins and on to the central circulation. The operator may then confirm with the patient that the flushing agent injection caused no discomfort. The flushing agent may also be injected at the beginning of the injection to fully distend the veins. The flushing agent may also be injected at least partially concurrently with the radiopharmaceutical to promote flow of the radiopharmaceutical through the venous access site 1018 or other port to the central circulation system of the patient P and thus reduce dwell time or exposure time to the injected drug. The flushing agent may also be injected subsequent to injection of the radiopharmaceutical to ensure that all of the radiopharmaceutical which passes the valve 1014 is flushed out of the fluid path set 1010 and into the patient P, ensuring a complete injection of the radiopharmaceutical dosage.

While the system 1000 is described in general herein, further details of particular system components may be found in the description of corresponding components in U.S. Patent Application Publication No. 2008/0294096, U.S. Pat. Nos. 9,005,166, 7,713,232, U.S. Patent Application Publication No. 2021/0338922, International Patent Application Publication No. WO2015/126526, U.S. Patent Application Publication No. 2021/0055431, U.S. Pat. Nos. 9,002,438, 7,813,841, U.S. Patent Application Publication No. 2018/0296751, and U.S. Patent Application Publication No. 2019/0307949, the disclosures of all of which are hereby incorporated by reference in their entireties.

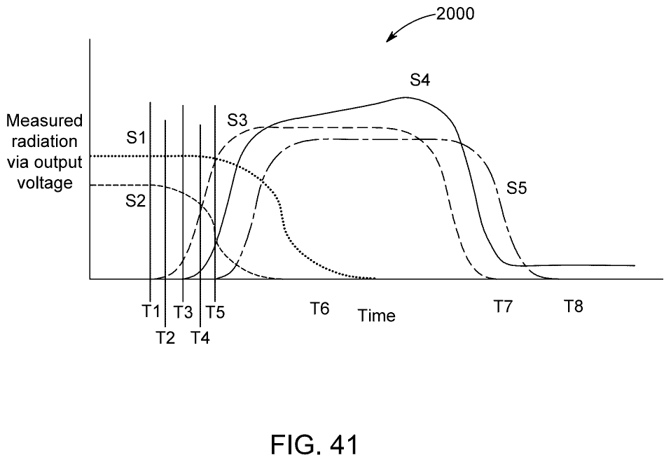

Referring now to FIG. 41, an exemplary graph 2000 of fluid delivery using the system 1000 of FIG. 40 is illustrated. In the graph 2000, time is shown on the x axis, and sensor output signal (e.g. voltage), corresponding to detected radiation, is shown on the y axis. It is to be understood the scales of x- and y-axis are not necessarily linear, and the units of the graphed values are arbitrary. The purpose of FIG. 41 is to illustrate a relationship between time and measured radiation during an arbitrary injection procedure, and the relative values may not be reflective of an actual injection procedure performed by the system 1000. Sensor outputs of actual radiation values will depend heavily upon the type and location of sensors and electronics used in the system 1000, as well as the geometries of the fluid path elements of the fluid path set 1010. The time relationship between various transitions in radiation values shown in FIG. 41 will likewise depend upon the fluid path element geometries and the flow rates of the radiopharmaceutical and flushing agent, which may change during an injection. Further, the depiction of the sensor output signals in graph 2000 assumes for simplicity that a “higher” sensor output corresponds to higher radiation, though this need not be true and the actual sensors used may exhibit, for example, an inverse relationship between output (e.g. voltage) and detected radiation. Nevertheless, graph 2000, along with all other graphs provided in the accompanying drawings, effectively illustrate principles of operation the system 1000.

With continued reference to FIGS. 40 and 41, time prior to T1 indicates a primed state of the system 1000 before an injection procedure has commenced. Thus, the output signal of sensor S1 and sensor S2 associated with the first fluid reservoir 1020A are substantially constant due to the presence of the radiopharmaceutical in the first fluid reservoir 1020A. The output signals of sensors S3, S4, S5 is a background or nominal value indicative of effectively zero radiation as no radiopharmaceutical has been advanced in the fluid path set 1010 beyond the T-connection 1012. At time T1, the piston actuator 1040 begins advancing the first plunger 1030A until the radiopharmaceutical upstream of the T-connection 1012 reaches a sufficient pressure to open the valve 1014. As the radiopharmaceutical is injected out of the first fluid reservoir 1020A, the output signal of sensor S1 begins to decrease (indicating a reduction in detected radiation). At time T2, the radiopharmaceutical has advanced within the fluid path set 1010 into the detection range of sensor S3. Thus, the output signal of the sensor S3 begins to increase (indicating an increase in detected radiation). Similarly, at time T3, the radiopharmaceutical has advanced to the filter 3000 and into the detection range of the sensor S4. Thus, the output signal of the sensor S4 begins to increase. At time T4, the first plunger 1030A has displaced sufficient volume of the radiopharmaceutical from the first fluid reservoir 1020A that the output signal from the sensor S2 begins to decrease. At time T5, the radiopharmaceutical has advanced into the detection range of the sensor S5. Thus, the output signal of the sensor S5 begins to increase. After a certain injection time, generally indicated within the time region T6, the radiopharmaceutical has reached a substantially constant, maximum flow through the fluid path set 1010 and the output signals from the sensors S3, S5 plateau (indicating a substantially constant level of detected radiation). The output signal of the sensor S4 continues to increase in time region T6, although a relatively slower rate than the initial increase at time T3, as radioactive isotopic impurities (or other filtered particles) accumulate in or on the filter 3000.

Once a desired amount of the radiopharmaceutical has been displaced from the first fluid reservoir 1020A, the piston actuator 1040 begins advancing the second plunger 1030B to deliver the flushing agent from the second fluid reservoir 1020B. As the flushing agent advances through the fluid path set 1010 and displaces the radiopharmaceutical, the output signals of the sensors S3, S5 decrease in time region T7 back to substantially zero (or near to their respective background outputs prior to time T1). The output signal of the sensor S4 likewise decreases as the flushing agent flushes radiopharmaceutical from the filter 300, but at least a portion of the trapped isotopic impurities trapped by the filter 3000 during delivery of the radiopharmaceutical remain. Thus, the output signal of the sensor S4 decreases to a substantially constant level indicative of the trapped isotopic impurities at time T8.

With continued reference to FIG. 41, the sensor output signals during an injection procedure can be used to monitor progress of the injection procedure. For example, the slope of the increase in output signal from the sensor S4 in the time region T6 may be used as an indicator of proper system functioning or a quality assurance indication. With a knowledge of the decay chain and half-lives of the isotopes of the radiopharmaceutical; the time at which the radiopharmaceutical was initially prepared with a given amount of the desired isotope; and a known, little, or no daughter or parent isotopes, it is possible to calculate the amount of isotope(s) expected to be present in the radiopharmaceutical. The expected rate of increase of daughter or parent isotopes in the filter 3000 as a function of the volume of the radiopharmaceutical that has flowed through the filter 3000 can thus be calculated by the system controller 9000. The expected rate of increase of daughter or parent isotopes can then be compared to the actual rate of increase of impurity isotopes (as measured by the sensor S4) to monitor the progress of the injection procedure. If the actual rate of increase of impurity isotopes deviates from the expected rate of increase of impurity isotopes by a predetermined amount or slope, the system controller 9000 may alert the operator to the situation, and either the operator or the system controller 9000 may take appropriate corrective action. As noted herein, the decay chain and half-lives of the isotopes of the radiopharmaceutical, the time at which the radiopharmaceutical was initially prepared with a given amount of the desired isotope, and other information used in calculating the expected rate of increase of impurity isotopes can be stored on the data element D1, retrieved by the system controller 9000 using the data element D1, or otherwise communicated to the system controller 9000 or computed by it from data stored in system controller 9000.

An increase in the output signal of sensor S4 at a lower rate than the calculated, expected increase may be an indication that an insufficient amount of daughter or parent is being removed by the filter 3000. An increase in the output signal of sensor S4 at a higher rate than the calculated, expected increase may indicate a problem with the initial measurement of radiation or that an undesirable amount of the radiopharmaceutical is being removed from the fluid path set 1010 by the filter 3000. In either case, the system controller 9000 may alert the operator to the situation, and either the operator or the system controller 9000 may take appropriate corrective action, for example pausing or terminating the injection procedure.

The output signal of the sensor S4 at time T8 may be also used as an indicator of proper system functioning or a quality assurance indication. For example the output signal of the sensor S4 at time T8 may be used to determine whether the appropriate amount of isotopic impurity was removed and that the appropriate amount of radiopharmaceutical was delivered to the patient (or to an interim pump or container as shown and described in connection with FIGS. 42, 43, and 54). The expected amount of isotopic impurity may be derived from known information, such as the decay chain and half-lives of the isotopes of the radiopharmaceutical, and the time at which the radiopharmaceutical was initially prepared with a given amount of the desired isotope.

In embodiments in which the radiopharmaceutical is supplied from a generator, for example a rubidium, gallium or technetium generator, the output signal of the sensor S4 may be used to identify a breakthrough of the parent isotope from the generator. In particular, too high a rate of increase in the output signal of the sensor S4 within the time region T6 or too high a residual output signal of the sensor S4 within the time region T8 may indicate breakthrough of the parent isotope from the generator. The system controller 9000 may be configured to generate an alert informing the operator to stop using that generator. Examples of systems including generators for producing radiopharmaceuticals include U.S. Pat. No. 7,813,841, U.S. Patent Application Publication No. 2018/0296751, and U.S. Patent Application Publication No. 2019/0307949, the disclosures of which are hereby incorporated by reference in their entireties.

Various types of sensors may be used as the sensors S1-S5. Examples include, but are not limited to, diode detectors, Geiger counters, solid state pulse counters, ion chambers, scintillators with photodiodes (e.g. avalanche photodiodes), and SPECT or PET cameras. Alternatively, the sensors S1-S5 may be more complex devices, such as survey meters, electronic dosimeters, and various commercial offerings from Lucerno Dynamics®. Further details of some of these types of sensors are described in U.S. Pat. No. 9,002,438 and U.S. Patent Application Publication No. 2021/0055431, the disclosures of which are hereby incorporated by reference in their entireties. The method of operation of the sensors S1-S5 may be of various complexity, including binary sensing, count or count rate sensing, dose or dose rate sensing, or energy or spectrum sensing. In some embodiments, any of the sensors S1-S5 may be an energy discriminating sensor, which by measuring the energy of the radiation may be able to quantify and differentiate the radiation measurements of the one or more of the isotopes involved. The sensors S1-S5 may be configured to detect various types of radiation pertinent to the injected radiopharmaceutical, such as alpha radiation, gamma radiation, neutrons, and beta radiation (either directly or via bremsstrahlung). The sensors S1-S5 may have detection ranges, i.e. sensitive areas, corresponding to a surface under the sensor, a segment of tube of known volume, or an entire fluid path element (e.g. the filter 3000). The sensors S1-S5 need not all be of the same type and configuration, and in fact it may be beneficial to use different types/configurations of sensors to measure radiation at different locations in the system 1000.

While the embodiment shown in FIG. 40 includes five sensors, additional sensors may be used for redundancy to improve safety and reliability, or to measure radiation at additional locations of interest. Alternatively, fewer than five sensors may be utilized. For example, in some embodiments, the sensors S1, S2 associated with the first fluid reservoir 1020A may be eliminated by relying on the initial calibration and content information of the first fluid reservoir 1020A. As noted herein, such information may be included in the data element D1 (or can be retrieved using the data element D1), allowing the system controller 9000 to implement or derive this information. In some embodiments, the sensors S3, S5 may be eliminated and only the sensor S4 used to detect radiation in the vicinity of the filter 3000. Alternatively, the sensor S4 may be eliminated, and the amount of isotope removed from the fluid path set 1010 by the filter 3000 may be computed based upon the output signals of sensors S3, S5, and a known flow rate of the fluids passing through the filter 3000. As mentioned elsewhere, the radiation in the radiopharmaceutical can be calculated from the initial calibration of the radiopharmaceutical in the radiopharmacy and the time since that calibration. While reduction in the number of sensors is possible for the reasons discussed above, multiple sensors can provide redundancy and the ability to detect failures that cannot be anticipated or detected by calculation. Examples of such failures includes leaks or occlusions in system components, a malfunction of one of the fluid delivery devices (e.g. the plunger 1030A, 10303B or the piston actuator 1040), or other failure of an element of the fluid path set 1010. As noted above, any of the sensors S1-S5 may be an energy discriminating sensor, which by measuring the energy of the radiation may be able to differentiate and quantify the radiation measurements of the one or more of the isotopes involved. This has the benefit of providing further information to reduce the number of sensors needed or to increase the reliability of the computations, system behaviors, assessment of filtration/removal, and/or ultimately the quality of the radiopharmaceutical being output by the system.

Referring now to FIG. 42, another embodiment of a fluid injector system 1000 may include an interim pump or fluid reservoir 1020C into which the prescribed dose of radiopharmaceutical and flushing agent is injected from the first and second fluid reservoirs 1020A, 1020B prior to delivery to the patient. In the event of a fault in the dosage of radiopharmaceutical delivered, the faulty dose is contained within the interim fluid reservoir 1020C and removed therefrom by the system 1000 or the operator without being delivered to the patient. The interim fluid reservoir 1020C illustrated in FIG. 42 is a syringe, driven by a plunger 1030C in the same manner that the first and second fluid reservoirs 1020A, 1020B are driven by respective plungers 1030A, 1030B. The plunger 1030C associated with the interim fluid reservoir 1020C may be controlled by the same piston actuator 1040 as the plungers 1030A, 1030B, or by an independent piston actuator. A pair of radiation sensors S1′, S2′ may be associated with the interim fluid reservoir 1020C and may be analogous in functionality to the radiation sensors S1, S2 associated with the first fluid reservoir 1020A. The system controller 9000 may determine and verify the dose or concentration of the radiopharmaceutical delivered to interim fluid reservoir 1020C from radiation measurements taken by the sensors S1′, S2′. Based on radiation measurements taken by the sensors S1′, S2′, the system controller 9000 can detect a fault in the dose delivered to the interim fluid reservoir 1020C. In the event such a fault is detected, the system controller 9000 may abort the injector procedure and alert the operator that the interim fluid reservoir 1020C should be removed and discarded. If no fault is detected, the system controller 9000 may proceed with the injection procedure by actuating the plunger 1030C to inject the dose from the interim fluid reservoir 1020C into the patient P.

With continued reference to FIG. 42, the interim fluid reservoir 1020C may be at least partially encapsulated by a radiation shield 2020′ analogous to the shield 2020 associated with the first fluid reservoir 1020A. A data element D1′ may also be associated with the interim fluid reservoir 1020C, analogous to the data element D1 associated with the first fluid reservoir 1020A.

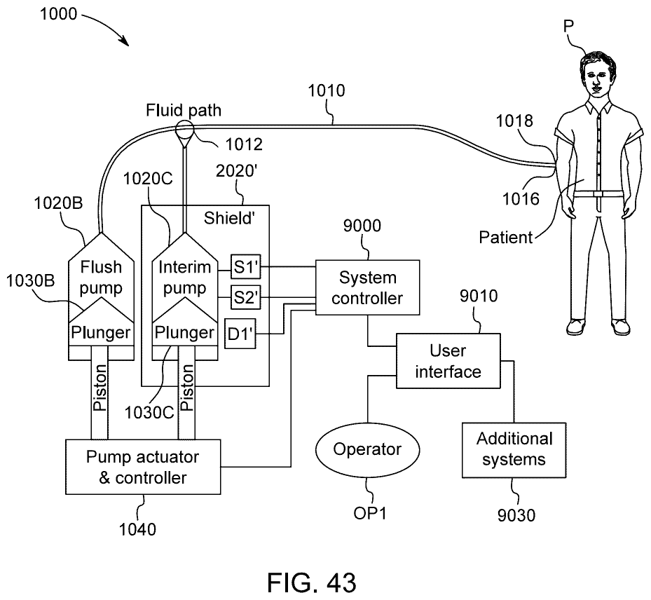

Referring now to FIG. 43, another embodiment of a fluid injector system 1000 may include an interim pump or fluid reservoir 1020C into which the prescribed dose of radiopharmaceutical and flushing agent is injected from the fluid reservoir 1020B prior to delivery to the patient. In the event of a fault in the dosage of radiopharmaceutical delivered, the faulty dose is contained within the interim fluid reservoir 1020C and removed therefrom by the system 1000 or the operator without being delivered to the patient. The interim fluid reservoir 1020C illustrated in FIG. 43 is a syringe, driven by a plunger 1030C in the same manner that the fluid reservoir 1020B is driven by respective plunger 1030B. The plunger 1030C associated with the interim fluid reservoir 1020C may be controlled by the same piston actuator 1040 as the plunger 1030B, or by an independent piston actuator. A pair of radiation sensors S1′, S2′ may be associated with the interim fluid reservoir 1020C and may be analogous in functionality to the radiation sensors S1, S2 associated with other embodiments of the invention. The system controller 9000 may determine and verify the dose or concentration of the radiopharmaceutical delivered to interim fluid reservoir 1020C from radiation measurements taken by the sensors S1′, S2′. Based on radiation measurements taken by the sensors S1′, S2′, the system controller 9000 can detect a fault in the dose delivered to the interim fluid reservoir 1020C. In the event such a fault is detected, the system controller 9000 may abort the injector procedure and alert the operator that the interim fluid reservoir 1020C should be removed and discarded. If no fault is detected, the system controller 9000 may proceed with the injection procedure by actuating the plunger 1030C to inject the dose from the interim fluid reservoir 1020C into patient P.

With continued reference to FIG. 43, the interim fluid reservoir 1020C may be at least partially encapsulated by a radiation shield 2020′ analogous to the shield 2020 associated with the first fluid reservoir 1020A. A data element D1′ may also be associated with the interim fluid reservoir 1020C, analogous to the data element D1 associated with other embodiments of the invention.

Referring now to FIG. 44, another embodiment of a fluid injector system 1000 includes a bulk source 1012A of the radiopharmaceutical to be administered to the patient P, or to multiple patients. The bulk source 1012A may be at least partially encased by a radiation shield 2220, analogous to the shield 2020 associated with the first fluid reservoir 1020A. The bulk reservoir 1012A may be storage container 200 with sufficient volume for multiple doses. A valve Va, such as a stopcock, may be opened by the system controller 9000 to allow the first plunger 1030A to pull radiopharmaceutical from the bulk source 1012A into the first fluid reservoir 1020A. The volume of the radiopharmaceutical dose pulled into the first fluid reservoir 1020A may be calculated based on the initial dose and volume or concentration of radiopharmaceutical in the bulk source 1012A and the time of measurement of the initial dose and volume or concentration. Knowing the decay constants of the isotope or isotopes in the radiopharmaceutical, the volume of radiopharmaceutical to be delivered can be calculated based on the decay from the time the radiopharmaceutical was initially measured to the current time. Any or all of this information may be stored on a data element D2 associated with the bulk source 1012A and accessible by the system controller 9000. Like the data element D1 associated with the first fluid reservoir 1020A, the data element D2 may be any type of data containing device, for example a printed label, a bar code, or an electronically readable device, for example a radiofrequency identification (RFID) tag. The sensors S1, S2 associated with the first fluid reservoir 1020A may be used as an alternative or confirmatory measurement of the dose of radiopharmaceutical pulled from the bulk source 1012A into the first fluid reservoir 1020A.

In the embodiment shown in FIG. 44, some of the components of the system 1000 are configured for single-patient use, and are disposed of after each patient, whereas other components are configured for multi-patient use. A connector C1 located in the fluid path set 1010 provides a hygienic connection between the multi-patient and single-patient components. Multi-patient components include the first fluid reservoir 1020A, the bulk fluid source 1012A, the valve Va, the connector C1, and the portion of the fluid path set 1010 upstream of the connector C1. The fluid path set 1010 downstream of the connector C1 are single-patient components. Example fluid path arrangements including single- and multi-patient components are described in patents U.S. Pat. No. 5,806,519 (ACDS total system), (ACDS Sterility Assurance) U.S. Pat. No. 5,569,181, (ACDS Multi-Patient Fluid Dispensing) U.S. Pat. No. 5,843,037, (ACDS Closed Loop Info Path 2) U.S. Pat. Nos. 5,920,054, 6,385,483 (ACDS Patient Specific Dosing), U.S. Pat. Nos. 8,926,569, 9,855,390, 9,173,995, 9,700,670, 9,480,797, 10,039,889, 10,512,721, and 10,668,221, the disclosures of all of which are hereby incorporated by reference in their entireties. The single-use fluid path set 1010 may include two check valves (not shown) somewhere along its length as a cross contamination prevention mechanism. The connector C1 may be a swabbable valve or a connector as described in International Patent Application Publication Nos. WO 2013/059563A1 and WO2014/210418A1, the disclosures of which are hereby incorporated by reference in their entireties. The single-use fluid path set 110 may extend all the way upstream and include fluid reservoir 1020A. In this case the connector C is on the fluid path between the bulk fluid reservoir 1012A and the, in this case, single patient fluid reservoir 1020A. Other components of the fluid injector system 1000 of FIG. 44 which are not particularly described are assumed to be similar to corresponding components of the embodiment of FIG. 40. In some embodiments, the system 1000 shown in FIG. 44 may include other components from FIG. 40 not shown for simplicity, such as the filter 3000 and associated sensors S3-S5, and the second fluid reservoir 1020B containing the flushing agent.

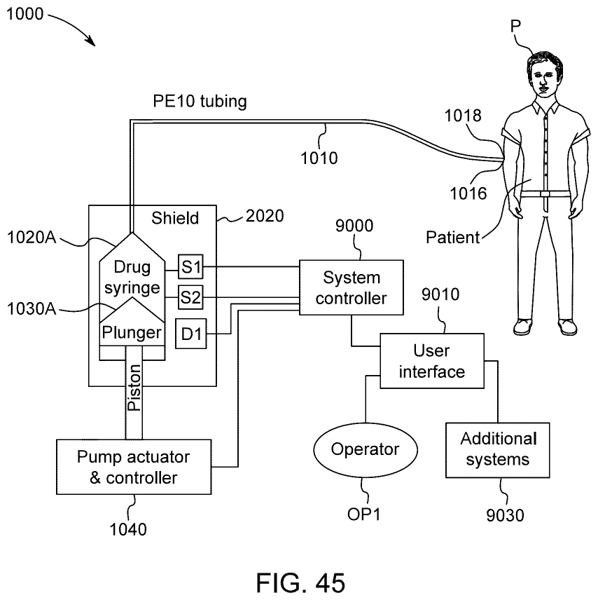

Referring now to FIG. 45, another embodiment of a fluid injector system 1000 is configured for single-patient use. The volume of radiopharmaceutical to be delivered may be computed based upon the initial dose and volume or concentration in the first fluid reservoir 1020A, decay corrected to provide the prescribed dose to the patient. The sensors S1, S2, may optionally be used to determine the dose or concentration in the first fluid reservoir 1020A. As in the embodiment of FIG. 40, the system controller 9000 may be configured to determine the volume of radiopharmaceutical in the first fluid reservoir 1020A based upon the position of the first plunger 1030A within the first fluid reservoir 1020A. Additional aspects of this calculation are described in U.S. Pat. No. 10,016,618, the disclosure of which is hereby incorporated by reference in its entirety. In some embodiments, the system 1000 shown in FIG. 45 may include other components from FIG. 40 not shown for simplicity, such as the filter 3000 and associated sensors S3-S5.

With continued reference to FIG. 45, the system controller 90000 may be configured to calculate the volume of radiopharmaceutical to deliver to the patient P, and thus the required travel distance of the first plunger 1030A. The calculation of the volume of radiopharmaceutical to deliver includes the volume needed to fill the fluid path set 1010 between the first fluid reservoir 1020A and the patient P. The length and volume of the fluid path set 1010 may be known, or may be obtained for example by scanning a data element associated with the fluid path set 1010. In order to reduce radiopharmaceutical waste, the volume of the fluid path set 1010 may be minimized by using small-diameter tubing. For example, the fluid path set 1010 may be constructed from PE10 tubing having an inner diameter of approximately 0.28 millimeters (0.011 inches).

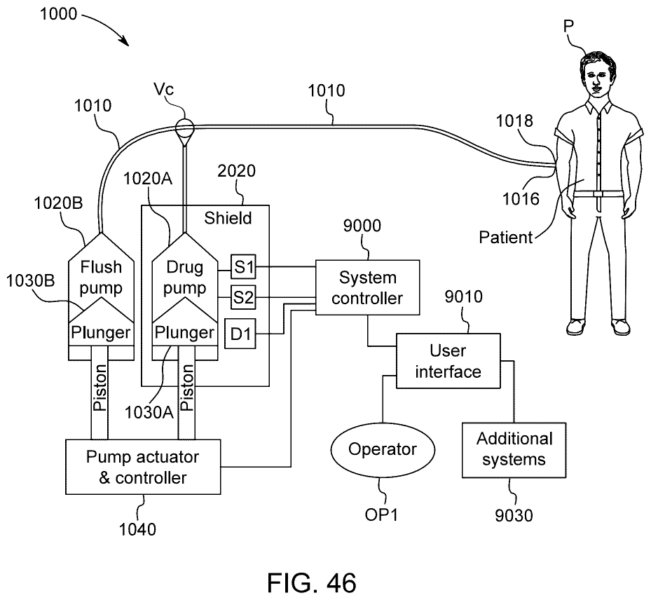

FIG. 46 illustrates another embodiment of the fluid injector system 1000 similar to the embodiment of FIG. 45, but including a second fluid reservoir 1020B containing a flushing agent. The flushing agent may, for example, be injected to prime the fluid path set 1010, as described herein with reference to the embodiment of FIG. 40. The flushing agent may also be injected before the radiopharmaceutical as a test injection to ensure system components are connected as intended. The flushing agent may also be injected at least partially concurrently with the radiopharmaceutical to promote flow of the radiopharmaceutical through the venous access site 1018 or other port to the central circulation system of the patient P. The flushing agent may also be injected subsequent to injection of the radiopharmaceutical to ensure that all of the radiopharmaceutical which passes a valve Vc, which may be a check valve, crack pressure valve, or stopcock, is flushed out of the fluid path set 1010 and into the patient P, ensuring a complete injection of the radiopharmaceutical. In some embodiments, the system 1000 shown in FIG. 46 may include other components from FIG. 40 not shown for simplicity, such as the filter 3000 and associated sensors S3-S5.

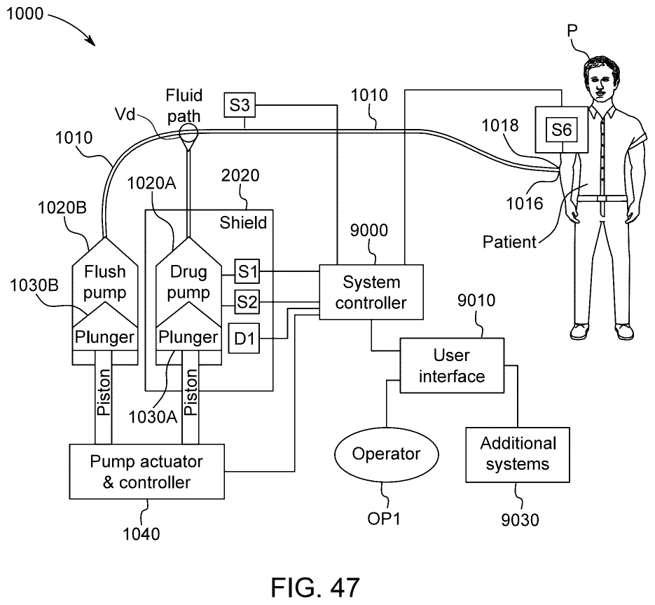

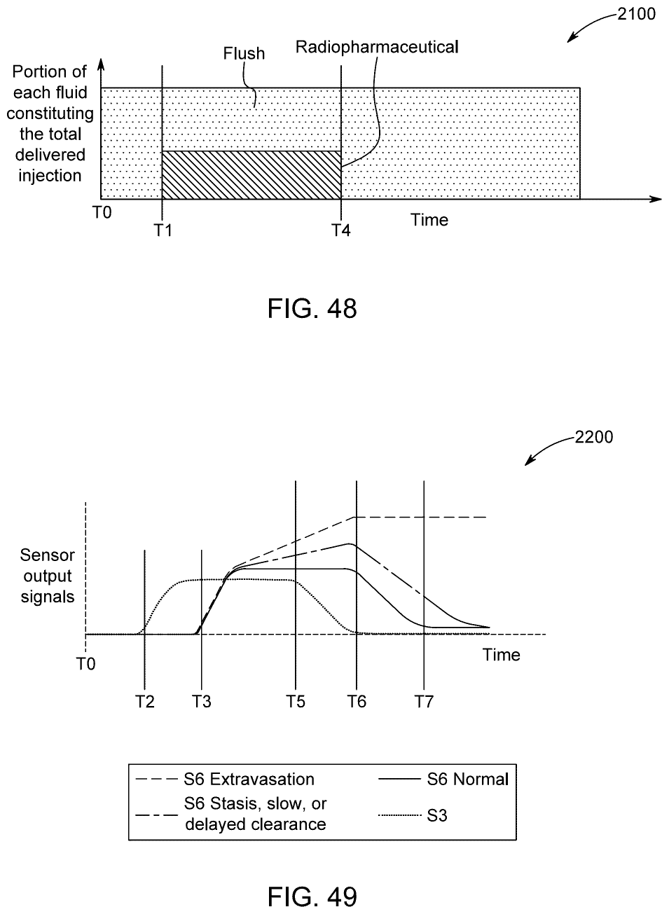

FIG. 47 illustrates another embodiment of the fluid injector system 1000 which includes at least one radiation sensor S6 associated with the patient P, for example in proximity to the venous access site 1018 where the radiopharmaceutical is injected. In some embodiments, the system 1000 may also include the sensor S3 (and/or other sensors S4, S5) as described herein with reference to FIG. 40. The sensor S6 is used to detect conditions such as retention of radiopharmaceutical at or near and the venous access site 1018, and/or to detect extravasation of the radiopharmaceutical into surrounding tissue of the patient P. Further detail of the process by which retention and extravasation is described herein with reference to FIGS. 48-53. In some embodiments, the system 1000 shown in FIG. 47 may include other components from FIG. 40 not shown for simplicity, such as the filter 3000.

FIGS. 48 and 49 illustrate an example use of the system 1000 of FIG. 47. FIG. 48 shows a graph 2100 of fluid injection over time. Time, in arbitrary units, is graphed on the x axis, and the portion each fluid constituting the total delivered injection is graphed on the y axis. Flushing agent is represented by the dotted area, and radiopharmaceutical is represented by the crosshatched area. Beginning at time TO and proceeding until time T1, an initial injection phase is performed using entirely the flushing agent to ensure that the system 1000 is connected and functioning as intended and to fill the patient's veins. During the initial injection phase, the operator may place his or her fingers on the patient P near the venous access site 1018 (see FIG. 47) to feel the flow and manually verify that the flow rate is sufficiently high. Alternatively or in addition, the operator may palpate for and confirm that that there is no swelling under the skin of the patient which might indicate an extravasation of the flushing agent. A good flow out the injected vessel may also be confirmed by the lack of significant back pressure to the second fluid reservoir 1020B during the test injection.

With continued reference to FIG. 48, at time T1, injection of the radiopharmaceutical is initiated and the radiopharmaceutical begins flowing through the fluid path set 1010. In some embodiments, the injection at this stage be entirely radiopharmaceutical, or, as shown in FIG. 48, the injection may be a mix of radiopharmaceutical and flushing agent. Simultaneous injection of a flushing agent may, for example, promote flow of the radiopharmaceutical through the fluid path set 1010 and the patient P to ensure rapid and thorough delivery of the radiopharmaceutical. Simultaneous injection of flushing agent may, for example, ensure that the radiopharmaceutical is flushed out of the venous access site 1018 and into the central circulation of the patient P. Simultaneous injection of flushing agent may also reduce any harmful effects that the radiopharmaceutical may have on the vessels of the patient P as it passes through them in a more concentrated form. At time T4, injection of the radiopharmaceutical is completed and the injection proceeds with substantially 100% flushing agent to flush the radiopharmaceutical from the fluid path set 1010 and the veins of patient P. In some embodiments, particularly for imaging procedures, the volumetric flow rate of the flushing agent during the flushing operation is substantially the same as the total volumetric flow rate during radiopharmaceutical injection (inclusive of the volumetric flow rate of any flushing agent delivered simultaneously with the radiopharmaceutical). Further details of the relationship between volumetric flow rates during drug injection and subsequent flushing is described in U.S. Pat. No. 10,933,186, the disclosure of which is hereby incorporated by reference in its entirety. For a purely therapeutic procedure, maintaining a constant volumetric flow rate between the injection and flushing phases is not necessarily critical, but may still be desirable.

Referring now to FIG. 49, a graph 2200 of sensor output signals for the sensors S3, S6 shown in FIG. 47 is shown on the same timeline as the graph 2100 of FIG. 48. At time T2, which occurs sometime after initiation of the radiopharmaceutical injection at time T1 of FIG. 48, the radiopharmaceutical has advanced within the fluid path set 1010 into the detection range of sensor S3. Thus, the output signal of the sensor S3 begins to increase (indicating an increase in detected radiation). After this initial rise, the output signal of the sensor S3 subsequently plateaus as the fluid path set 1010 in the detection range of the sensor S3 is filled with the maximum concentration of the radiopharmaceutical. As described herein, the system controller 9000 may be configured to determine the expected radiation using known information such as volume of the fluid path set 1010 and flow rate, concentration, and decay rate of the radiopharmaceutical. If the radiation detected by the sensor S3 deviates sufficiently from the time course expected for the radiation, the system controller 9000 may indicate an error and alert the operator. This condition could be caused by a failure of the piston actuator 1040 or a leak in the fluid path set 1010, for example or a mistake in the initial calibration date, for example stored in D1.

With continued reference to FIG. 49, at time T3, the radiopharmaceutical reaches the patient P and is within the detection range of the sensor S6. The output signal of the sensor S6 begins to rise, indicating the radiopharmaceutical is being delivered to the patient P. If after a predetermined time interval the system controller 9000 has not received an output signal from the sensor S6 indicative of the radiopharmaceutical reaching the patient P, the system controller 9000 may halt the injection procedure and alert the operator to the possibility of a fault. At time T5, which occurs sometime after time T4 of FIG. 48, the output signal of the sensor S3 decreases as the radiopharmaceutical is flushed out of the fluid path set 1010 and thus out of the detection range of the sensor S3.

With continued reference to FIG. 49, during a normal injection procedure, the output signal of the sensor S6 follows the time course of the solid line labeled “S6 normal”. In particular, the output signal of the sensor S6 rises as the radiopharmaceutical reaches the patient P and thus is within the detection range of the sensor S6. The output signal of the sensor S6 then plateaus once the radiopharmaceutical reaches a maximum prescribed flow, and subsequently decreases to a whole body distribution value beginning at T6. Once the radiopharmaceutical has been completely flushed from the venous access site 1018, at time T7, the output signal from the sensor S6 may remain at the whole body distribution value. As the sensor S6 is located downstream of the sensor S3, the output signal of the sensor S6 generally rises, plateaus, and decreases at a time delay relative to the sensor S3. The exact amplitude of the output signal of the sensor S6 depends upon many factors such as the flow rate and fluid path element volumes, the position of the sensor S6 relative to the venous access site 1018 or other patient vessels or part, the path of the vessels or port, and the depth of the vessels or port.

With continued reference to FIG. 49, the system controller 9000 may monitor the output signal of the sensor S6 to detect anomalies in delivery of the radiopharmaceutical. For example, the output signal of the sensor S6 may be used to determine that an extravasation or partial extravasation has occurred. In the event of an extravasation, the output signal of the sensor S6 may follow the time course of the dashed line labeled “S6 extravasation”. Following the expected initial rise in the output signal corresponding to the radiopharmaceutical initially reaching the patient, the output signal of the sensor S6 will continue to rise during the injection rather than plateau as expected. Additionally, the output signal of the sensor S6 will exhibit little or no decrease once the injection of radiopharmaceutical has ended at time T6. The lack of decrease in the output signal of the sensor S6 after time T6 indicates that the radiopharmaceutical is not being flushed from the patient by the injection of the flushing agent. Further, the output signal of the sensor S6 may maintain the same magnitude after T7, when the radiopharmaceutical would be expected to have been entirely flushed from the venous access site 1018, indicating that the radiopharmaceutical has extravasated into the tissue around the venous access site 1018. The amount of extravasated radiopharmaceutical may be the entire volume injected or a fraction of the injected volume. Additional methodologies of extravasation monitoring that may be implemented into the system 1000 are described in International Patent Application Publication No. WO 2021/222771, the disclosure of which is hereby incorporated by reference in its entirety.

With continued reference to FIG. 49, the system controller 9000 may also monitor the output signal of the sensor S6 to detect retention (i.e. stasis or delayed clearance) of some or all of the radiopharmaceutical in the patient. In the case of retention, most or all of the radiopharmaceutical is successfully injected into the patient's vessels or port, but the native flow in the vessel and the flow rate of the injection is insufficient to advance the radiopharmaceutical into the patient's central circulation. Thus, the radiopharmaceutical accumulates at the venous access site 1018, or at another location in the vessel. In this situation, the output signal of the sensor S6 may follow the time course of the dot-dash line labeled “S6 Stasis, slow, or delayed clearance” in FIG. 49. The output signal of the sensor S6 will increase during the injection even after the radiopharmaceutical reaches maximum flow, similar to what is observed during an extravasation. Though the slope of the increase shown in FIG. 49 is may be less for the retention case than the extravasation case as some drug does flow downstream to the centra circulation, it may not be possible in practice to distinguish between venous retention and extravasation based solely on the increase in output signal of the sensor S6. A slope that represents venous retention on one patient may be the same as the slop for extravasation on a different patient because of the many differences in, for example, the patients' arms, veins, and sensor placement. After the injection is completed, the output signal of the sensor S6 will decrease at time T6, as the radiopharmaceutical is flushed out the patient's vessels or port. However, the decrease in the output signal of the sensor S6 occurs more slowly than during a normal injection, and more rapidly than during a full extravasation, which may have almost no decrease. In addition, there may be cases, not shown, in which some radiopharmaceutical extravasates and some radiopharmaceutical is just slow to leave the vein. After determining that an extravasation or retention of the radiopharmaceutical has occurred, the system controller 9000 may alert the operator so that further actions can be taken to assess the patient's situation and corrective action can be begun if necessary.

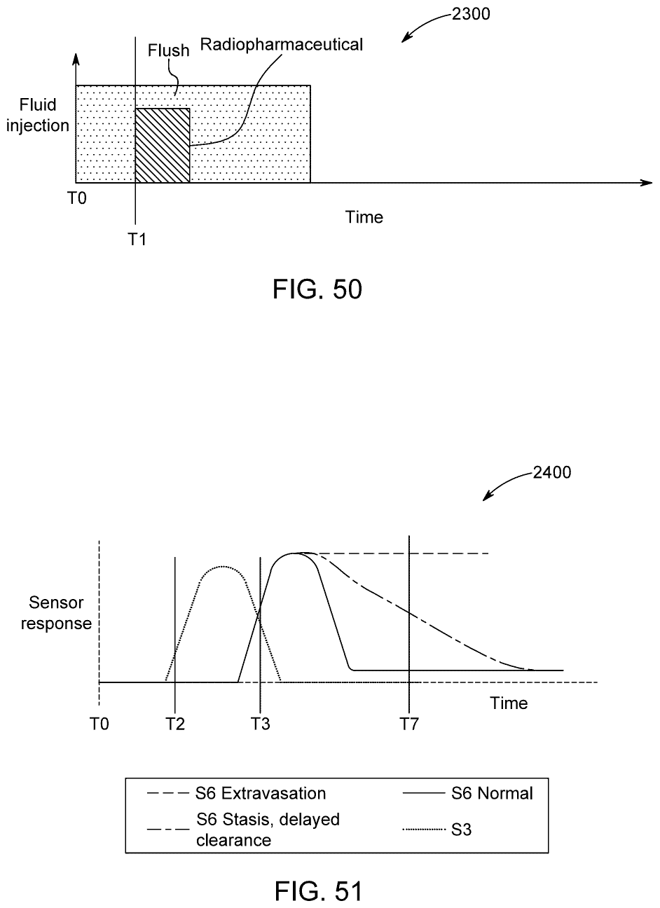

Referring now to FIGS. 50-53, the sensors S3, S6 may also be used to identify an extravasation or retention (i.e. delayed clearance) before the full dosage of the radiopharmaceutical has been delivered so that corrective actions may be taken, and/or the injection procedure may be halted appropriately. FIGS. 50 and 51 illustrate graphs 2300, 2400, respectively, of fluid injection over time and sensor response over time according for one such embodiment of operating the system of FIG. 47. A test injection of the radiopharmaceutical, for example a predetermined amount between 5% and 20% of the total dose, is given and the response to that test injection is assessed before delivering the remainder of the radiopharmaceutical dose. This test volume is exemplary and is chosen to be just large enough to be reliably measured by the sensors and so will depend upon the specific sensors chosen. It is desirable to keep the volume as low as can be reliably measured by the sensors to minimize the harm to the patient if an extravasation happens. As with the embodiment shown in FIG. 48, beginning at time T0 and proceeding until time T1, a test injection is performed using entirely the flushing agent to ensure that the system 1000 is connected and functioning as intended. At time T1, injection of the radiopharmaceutical begins, again analogous to the embodiment shown in FIG. 48. At time T2, the output signal of the sensor S3 increases as the radiopharmaceutical enters the detection range of the sensor S3. At time T3, the output signal of the sensor S6 increases as the radiopharmaceutical enters the patient P and corresponding detection range of the sensor S6. Because only the test injection (e.g. 5%-20% of the total dose) of the radiopharmaceutical is initially being delivered, the size of the radiopharmaceutical bolus within the fluid path set 1010 and/or the patient P may not be sufficiently large to cause a plateau in the output signals of the sensors S3, S6 as is observed during a full-dose injection (as shown in FIGS. 48 and 49). Rather, the output signals of the sensors S3, S6 may reach a peak value and then immediately begin to decrease as the radiopharmaceutical is flushed from the system 1000, as shown in FIGS. 50 and 51. In other embodiments, the plateau of the sensor output signals may still occur, but the plateau may be present for less time compared to the full-dose injection shown in FIGS. 48 and 49.

After the test injection of the radiopharmaceutical has been injected, the flushing agent is then injected to flush the radiopharmaceutical from the test injection, and the output signals of the sensor S3, S6 are monitored for indications of extravasation and/or retention of the radiopharmaceutical. In the absence of extravasation or retention, the output signal of the sensor S6 decreases to a whole body distribution value after the test injection of the radiopharmaceutical has been flushed, for example by time T7. This behavior is shown by the time course of the solid line labeled “S6 normal” in FIG. 51. In comparison little or no significant decrease in the output signal of the sensor S6 at time T7 indicates the existence of an extravasation condition, as shown by the time course of the dashed line labeled “S6 extravasation”. Similarly, a decrease in the output signal of the sensor S6 at time T7 that is less rapid than would be expected from a normal injection indicates the existence of a retention condition, as shown by the time course of the dot-dash line labeled “S6 stasis, delayed clearance”.

The system controller 9000 may use predetermined thresholds on the rate of decrease of the output signal of the sensor S6 to differentiate between normal injections, retention (i.e. delayed clearance), and extravasations. The system controller 9000 may alert the operator accordingly. In the case of normal flow through the system, the system controller 9000, either automatically or upon input from the operator, may proceed with injection of the remainder of the dose of radiopharmaceutical. If retention (i.e. delayed clearance) is detected, the system controller 9000, either automatically or upon input from the operator, may still proceed with injection of the remainder of the dose of radiopharmaceutical, but modifications to the injection procedure may be implemented to address the retention condition. For example, the system controller 9000 may reduce the pharmaceutical injection flow rate, while maintaining the total volumetric flow, to promote clearance and reduce accumulation of the radiopharmaceutical in the patient's vessels. Alternatively, the system controller 9000 may increase the flush agent injection rate to promote clearance and reduce accumulation of the radiopharmaceutical in the patient's vessels. If extravasation is detected, the system controller 9000, either automatically or upon input from the operator, may abort the injection procedure. The operator may then attempt to find a new venous access site 1018, or wait until a different time or day to retry the injection.

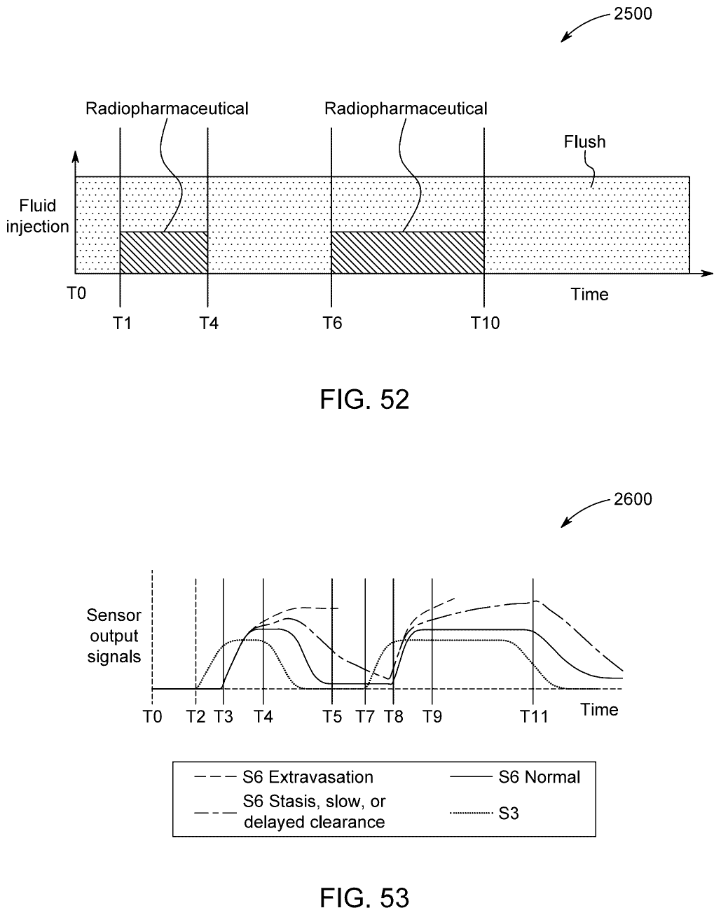

FIGS. 52 and 53 show graphs 2500, 2600 illustrating another embodiment of the operation of the system 1000 of FIG. 47. As shown in the graph 2500 of FIG. 52, delivery of the radiopharmaceutical is divided into multiple stages. A first stage of radiopharmaceutical injection is performed between times T1 to T4, and a second stage of radiopharmaceutical injection is performed between times T6 to T10. The volume of radiopharmaceutical injected during the first stage may be large enough to be reliably detected by the sensors and low enough that no patient harm would be caused even in the event of an extravasation. While the embodiment of FIGS. 52 and 53 show two stages of pharmaceutical injection, any number of stages may be used. As with the embodiments described in FIGS. 48-51, a short injection of flushing agent starts at time T0. Then the first stage of radiopharmaceutical injection begins at time T1, which optionally includes the injection of a flushing agent injected simultaneously with the radiopharmaceutical. As the radiopharmaceutical advances through the fluid path set 1010 into the detection range of the sensor S3, at time T2, the output signal of the sensor S3 begins to rise before plateauing in a similar manner as described in connection with FIG. 49. Likewise, at time T3, the output signal from the sensor S6 begins to rise as the radiopharmaceutical advances to the patient P and into the detection range of the sensor S6. At time T4, the first stage of radiopharmaceutical injection is completed. If the output signal of the sensor S6 has plateaued and is no longer rising, then the system controller 9000 may determine that the injection is proceeding normally and may continue with the next radiopharmaceutical injection at time T6.

However, if at time T4, the output signal of the sensor S6 is continuing to rise, the system controller 9000 may determine that either a retention (i.e. slow clearance) or extravasation condition is present because the radiopharmaceutical is not being flushed from the detection range of the sensor S6 by the flow of the flushing agent injected after time T4. As discussed herein, it is difficult to a priori determine whether a sustained increase in the output signal of the sensor S6 after time T4 is the due to extravasation or slow clearance. To make a definitive determination, the system controller 9000 may inject flushing agent between time T4 and T6 while monitoring the output signal of the sensor S6. A decrease in the output signal of the sensor S6 during the injection of flushing agent between times T4 and T6 indicates that the radiopharmaceutical is being flushed from the patient P and therefore the present condition is retention rather than extravasation. A plateau in the output signal of the sensor S6 during the injection of flushing agent between times T4 and T6 indicates that the radiopharmaceutical is not being flushed and, therefore, extravasation has occurred.

If an extravasation condition is determined to the present, the system controller 9000 may abort the injection procedure and/or alert the operator. If retention is determined to be present, the system controller 9000 may alert the operator and, either automatically or under operator guidance, proceed with the injection of the second stage of radiopharmaceutical at time T6. To mitigate further retention of radiopharmaceutical during the second stage of fluid injection, the system controller 9000 may modify the prescribed injection procedure by decreasing the injection flow rate of the radiopharmaceutical, and/or by increasing the simultaneous injection of the flushing agent, as described in connection with FIG. 51.

As the second stage of the injection proceeds, beginning at time T6, the system controller 9000 continues to monitor the output signal of the sensor S6 to monitor for extravasation. Even if extravasation did not occur during the first stage of the radiopharmaceutical injection, it is still possible for extravasation to develop in subsequent stages of radiopharmaceutical injection. At time T7, the output signal of the sensor S3 begins to rise as the radiopharmaceutical again enters the detection range of the sensor S3. Similarly, at time T8, the output signal of the sensor S6 begins to rise as the radiopharmaceutical again enters the detection range of the sensor S6. During a normal injection, the output signals of both the sensors S3, S6 plateau during maximum flow of the radiopharmaceutical. The system controller 9000 may monitor the output signal of the sensor S6 at a predetermined time, for example at time T9, for indications of extravasation. In particular, the system controller 9000 may compare the output signal of the sensor S6 at time T9 to the output signal of the sensor S6 at time T4. If retention was present at time T4, the system controller 9000 may record the rate of increase in the output signal of the sensor S6 and consider it as corresponding to retention. Thus, if the output signal of the sensor S6 increases at time T9, the system controller 9000 can compare the rate of increase at time T9 to the learned rate of increase at time T4. If the increase in the output signal of the sensor S6 at time T9 is similar or equal to the increase at time T4, the system controller 9000 can determine that the previous retention is occurring at T9 and may alert the operator and continue the injection. Conversely, if the increase in the output signal of the sensor S6 at time T9 is more rapid than the increase at time T4, the system controller 9000 can determine that extravasation is occurring at time T9. In some embodiments, the system controller 9000 may use a threshold based on the output signal at time T4 to distinguish between retention and extravasation at time T9. For example, the system controller 9000 may determine that extravasation has occurred at time T9 if the rate of increase of the output signal of the sensor S6 at time T9 exceeds that rate of increase of the output signal at time T4 by a predetermined percentage.

If the system controller 9000 determines that extravasation is occurring at time T9, or at any time thereafter, the system controller 9000 may pause, hold, or abort the injection and alert the operator until subsequent action can be determined. If the rate of increase of the output signal of the sensor S6 at time T9 is similar to or less than the rate of increase of the output signal at time T4, the system controller 9000 may proceed to the conclusion of the injection procedure.

An alternative method of using the sensor S6 to monitor the system 1000 for extravasation can also be understood from FIGS. 52 and 53. Instead of the dividing the radiopharmaceutical injection into a plurality of predetermined volumes separated by a flush, as previously described, the system controller 9000 may intend to deliver the entire volume of the radiopharmaceutical in a single dose. As in the previously-described embodiments, the output signal of the sensor S6 is expected to plateau at time T4 during a normal operation (i.e. no retention or extravasation is present). So long as the output signal of the sensor S6 remains plateaued as expected, the system controller 9000 proceeds to inject the entire dose of the radiopharmaceutical in a single stage (similar to the time curves shown in FIG. 49). An increase in the output signal of the sensor S6 at or after time T4, however, indicates that the radiopharmaceutical is not flowing through the patient's vessels as intended. To differentiate between an extravasation and retention (i.e. slow clearance), the system controller 9000 may pause injection of the pharmaceutical and continue with injection of only the flushing agent. At time T5, the system controller 9000 observes whether the output signal of the sensor S6 has plateaued or is decreasing. If the output signal of the sensor S6 remains plateaued time T5, or if the output signal has not decreased by a predetermined magnitude, percentage, etc., the system controller 9000 alerts the operator to a possible extravasation and stops the injection. In contrast, if the output signal of sensor S6 has decreased at time T5 by the predetermined magnitude, percentage, etc., the system controller 9000 may determine that retention, rather that extravasation, is occurring. The system controller 9000 may then resume the normal injection of the radiopharmaceutical, using the rate of rise of the output signal of the sensor S6 measured at T4 to assess whether a rise during the remainder of the injection, for example at T9, is within the expected range or if an extravasation (too fast a rise) or leak (too slow a rise or a lack of rise) may be occurring. If any fault is sensed, the system controller 9000 may alert the operator and the system controller 9000 may pause the injection automatically or on the command of the operator.

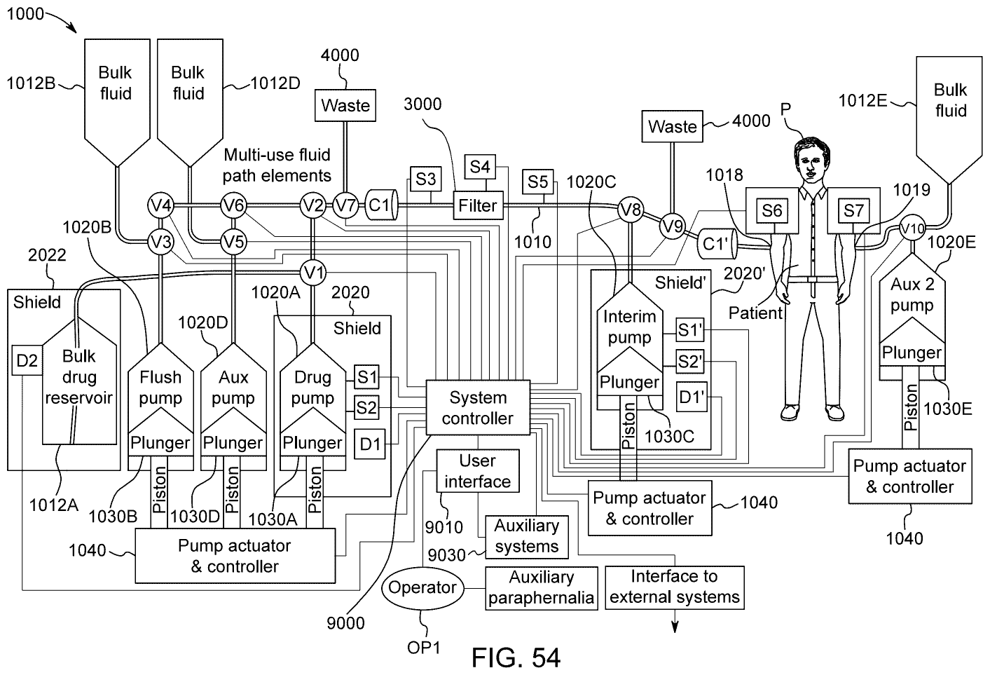

FIG. 54 is another embodiment of the system 1000 which combines features of all of the embodiments shown in FIGS. 40, 42, 43, 44, 45, 46, and 47. Included in the embodiment of FIG. 54 are the filter 3000 substantially as described in connection with FIG. 40 and the interim fluid reservoir 1020C and associated components substantially as described in connection with FIGS. 42 and 43. The system 1000 may further include one or more auxiliary fluid pumps or reservoirs 1020D, 1020E for injecting additional fluids, such as protectants, uptake promoters, amino acid mixtures, and the like. Injection from all of the fluid reservoirs 1020A-1020E is controlled by the system controller 9000, allowing the system 1000 to deliver of a number of fluids in a controlled sequence. Any of the fluids, e.g. the radiopharmaceutical, the flushing agent, and any additional/auxiliary fluids may be drawn into their respective fluid reservoirs 1020A, 1020B, 1020D, 1020E from respective bulk fluid sources 1012A, 1012B, 1012D, 1012E. The implementation of such bulk fluid sources is described herein with reference to FIG. 44. The system 1000 may include multi-patient to single patient connectors at various locations, for example connectors C1 and C1′ depending upon which fluid path elements are designed to be multi-patient and which are designed to be single patient.

Various valves V1-V10 may be located throughout the fluid path set 1010 and operated by the system controller 9000 to control the flow of fluid(s) through the various fluid path elements, fluid reservoirs 1020A, 1020B, 1020D, 1020E, and bulk fluid sources 1012A, 1012B, 1102D, 1012E. The valves V1-V10 may be active valves such as pinch valves or stepper motor rotary actuated stopcocks, passive valves such as stopcocks, or manually activated valves.

One of more of the auxiliary fluid reservoirs, for example the auxiliary fluid reservoir 1020E shown in FIG. 54, may be fluidly connected to a second venous access site 1019 of the patient distinct from the venous access site 1018 where the radiopharmaceutical from the first fluid reservoir 1020A is injected. This may be done, for example, in cardiac stress imaging where the steady flow of the stress agent from the fluid reservoir 1020E into the patient P must be maintained at a prescribed flow rate that should not be sped up or slowed down by the injection of the radiopharmaceutical. In another example, the auxiliary fluid or drug from the auxiliary fluid reservoir 1020D or 1020E may be an amino acid mixture to reduce the dose to the kidneys from the radiotherapy. In another example, the auxiliary fluid reservoir 1020D or 1020E may be a “cold” version of the targeting molecule that has no radioactive isotope bound to it. Alternating “hot” and “cold” versions of the targeting molecule can be used in receptor binding studies or as described in U.S. Pat. No. 10,016,618.

With continued reference to FIG. 54, the system 1000 may include a reference radiation sensor S7 located on the patient P away from the venous access site 1018. The sensor S7 may be on the patient's opposite limb, over the liver, or over a target organ such as the heart or kidneys. The system controller 9000 may be configured to compare the output signal of the reference radiation sensor S7, which would indicate the whole body distributed radiation, to the output signal of the sensor S6 to identify the presence of retention or extravasation near the sensor S6.

With continued reference to FIG. 54, the system 1000 may include one or more waste vessels 4000 for safely containing air from priming, radiopharmaceuticals, or other fluids for disposal. The waste vessels 4000 may be in fluid communication with the first fluid reservoir 1020A and interim fluid reservoir 1020C so that if a faulty dose of the radiopharmaceutical is detected, the system controller 9000 can redirect the faulty dose to the waste vessels 4000 via the valves V7, V9. The filled waste vessels 4000 can then be safely removed from the system 1000 and disposed of. The injection procedure may be restarted with a fresh dose of the radiopharmaceutical.

As described herein, the system controller 9000 of the any of the embodiments of the systems 1000 illustrated in FIGS. 40-54 may implement an injection procedure for delivering any of the fluids from the various fluid reservoirs 1020A-1020E to the patient P. During the delivery of the fluids, the system controller 9000 may be configured to receive output signals, corresponding to radiation measurements from the sensors S1-S7. Based on these radiation measurements, the system controller 9000 may be configured to make various determinations and halt or modify the injection procedure. In some examples, as described herein, the system controller 9000 may be configured to determine that an amount of radioactive particles in any of the fluid path elements or fluid reservoirs 1020A-1020E satisfies a predetermined threshold. Examples of such predetermined thresholds include a predetermined prescribed dosage of the radiopharmaceutical, a predetermined safe dosage of the radiopharmaceutical, a predetermined retention amount, and a predetermined rate of change of radioactivity over time.

In some examples, the system controller 9000 may be configured to determine a cumulative amount of radiopharmaceutical injected from the first fluid reservoir 1020A or the interim fluid reservoir 1020C. The system controller 9000 may make this determination based on the radiation measurements from one or more of the sensors S1-S7 and one or more injection parameters. Example injection parameters include an injection rate of the radiopharmaceutical, an injection volume of the radiopharmaceutical, an injection duration of the radiopharmaceutical, a half-life of the radiopharmaceutical, a decay chain of the radiopharmaceutical, an age of the radiopharmaceutical, a volume of the radiopharmaceutical, a concentration of the radiopharmaceutical, and an initial radioactivity of the radiopharmaceutical. During or after the injection, the system controller 9000 may compare the cumulative amount of the radiopharmaceutical injection to a prescribed dosage as a confirmation check. In some examples, the system controller 9000 may be configured to record, report to the user, or communicate to external systems any or all of the information related to the injection, including, for example, one or more injection parameters,

In some examples, the system controller 9000 may be configured to determine, based on the radioactivity measurement received from one of the sensors S3-S5, a residual level of radioactive particles in the radiation filter 3000 or in the fluid path set 1010. In some examples, the system controller 9000 may be configured to determine, based on the radioactivity measurement received from one or more of the sensors S1-S7, that the radiopharmaceutical is in chelation. In some examples, the system controller 9000 may be configured to determine an amount of radioactive particles suspended in the filter 3000 by comparing a radioactivity measurement from the sensor S3 and a radioactivity measurement from the sensor S5.

In some examples, the system controller 9000 may be configured to determine the presence of retention or extravasation in relation to the venous access site 1018 based on radiation measurements received from at least one of the sensors S3-S6. In particular, the system controller may come the radiation measurements from the sensor S3 in the fluid path set 1010 and the sensor S6 at the venous access site 1018. In some examples, the system controller 9000 may be configured to detect a blockage or leakage in the fluid path set 1010 based on radiation measurements received from at least one of the sensors S3-S6. In particular, the system controller 9000 may determine from a lack of radiation measurement at any of the sensors S3-S6 that the radiopharmaceutical has not reaches that sensor as intended, indicating a leak or blockage. Additionally, a significant increase the radiation measurement of any of the sensors S3-S5 in the fluid path set 1010 may indicate an accumulation of the radiopharmaceutical due to a blockage.

It is noted that the various embodiments of the system 1000 described herein are illustrated schematically, and the various components are positioned relative to one another in a manner to clearly illustrate the operation of the system 1000. However, the arrangement of components in actual practice may vary, and individual components may be stored in different rooms and even in totally different sites. For example, the fluid reservoirs 1020A, 1020C, 1020D, 1020E containing radioactive substances may be stored in a separate room from the patient P to avoid unnecessary radiation exposure to the patient P and operator. The system controller 9000 may include components such as servers and memory connected with wires and/or wirelessly to the at least one processor from remote locations. Further, the system controller 9000 may be connected to and share information with wearable devices such as smart phones, smart watches, etc.

The various fluid reservoirs 1020A-1020E have generally been described as syringe-piston-plunger devices, though any form of fluid pump and reservoir could be used. For example, any of the fluid reservoirs 1020A-1020E could include a bottle, bag, syringe, or tube with a peristaltic pump for fluid delivery or a pressurized reservoir. Fluid pumps such as peristaltic, diaphragm, and piston may be used. In general, positive displacement pumps may be used and non-positive displacement pumps combined with sufficiently accurate flow or volume meters may be used. As noted herein, the first fluid reservoir 1020A containing the radiopharmaceutical may alternatively be a generator for producing the radiopharmaceutical with fluid driven or drawn through it.

Embodiments of the system 1000 described herein may be used for injection of a wide variety of radiopharmaceuticals, particular details of which are described in the various patent documents incorporated by reference. Such radiopharmaceuticals may include, for example, various isotopes of technetium, thorium, actinium, radium, and rubidium. Depending on the isotope and injection procedure, prescribed dosages can range from fractions of a milliliter (mL) to 10s of milliliters, for example approximately 7 mL.

In light of the above, the various fluid delivery systems of the present disclosure permit for an improved method to treat various medical issues and, in particular, to methods and systems for radiotherapy delivery as radiotherapy is set to expand by a factor of up to about 10× in the near future. Given this potential increase in the need for radiotherapy to treat various medical conditions, there is a need to expand the places and people who can supervise and/or administration radiopharmaceuticals. Furthermore, the fluid delivery systems of the present disclosure may be designed to: (i) reduce the radiation exposure received by the operator/administrator; (ii) provide the operator/administrator a better awareness of the dose and/or dose rate of a radiopharmaceutical given to a particular patient; and/or (iii) provide for one or more separate filters, one or more separate sensors, and/or one or more separate controllers so that the fluid delivery systems of the present disclosure are able to handle any isotope-based radiopharmaceutical. Furthermore, the fluid delivery systems of the present disclosure may be designed to reduce the connections to be made and reduce, eliminate, or prevent the disconnections made after a procedure is competed to reduce the chance of leakage or transmission of any dangerous materials to the operator or the environment.

Additionally, the various fluid delivery systems of the present disclosure may present embodiments where dose calibrators may be eliminated as such dose calibrators can be expensive and/or onerous to maintain. Thus, in one instance, the various fluid delivery systems of the present disclosure permit are able to function with multiple syringes of various volumes between 5 mL-65 mL. In another instance, the various fluid delivery systems of the present disclosure may present embodiments where it enables an operator/administrator to deliver: (i) cytoprotective and other preparatory drugs; (ii) deliver sensitizing drugs; (iii) pause and restart an infusion mid treatment without loss of info and/or accuracy (e.g. a pause flushes various lines before opening); and/or (iv) detect one or more leaks.

In still another instance, the various fluid delivery systems of the present disclosure enable an operator/administrator to realize patient customized doses based on one or more factor including, but not limited to, weight, infusion rate, total volume, and/or to realize the ability to select a desired flow rate depending on one or more patient conditions and/or factors (e.g. proteins require slower infusions.) and/or supply a patient with more or less saline depending upon individual patient requirements and/or various treatment requirements.

In still another instance, the various fluid delivery systems of the present disclosure enable an operator/administrator a wide array of other types of drugs beyond radiopharmaceuticals including, but not limited to, one or more imaging drugs (e.g. CT contrast, MR contrast, ultrasound contrast, etc.), chemotherapy drugs, immune-regulatory drugs (e.g, drugs for one or more autoimmune disorders including, but not limited to, control Crohn's, ulcerative colitis, rheumatoid arthritis, Lupus, multiple sclerosis, etc.). In still another instance, the various fluid delivery systems of the present disclosure may be used to deliver: (i) Sirtex spheres for the treatment of solid tumors and the like; and/or (ii) prefilled single use patient doses.

In still another instance, the various fluid delivery systems of the present disclosure enable the use of any electronically readable sensor including, but not limited to, an electronic dosimeter, a Lucerno device, an ion chamber, a scintillator with photo diode or avalanche photo diode, etc. In still another instance, the various fluid delivery systems of the present disclosure enable radiation sensing, real-time or otherwise, of alpha, beta directly, beta braking radiation (e.g. deceleration radiation or bremsstrahlung), gamma, and/or neutrons. In still another instance, the various fluid delivery systems of the present disclosure utilize various radiation sensing modes via various methods and/or devices including, but not limited to, pulse count, current, pulse height (charge) measurement, Geiger counter, solid state pulse counter (CZT), diode current measurement, a suitable type of gamma ray spectrometer, Lucerno extravasation detector, a ThermoFischer Scientific sensor, a Berkley nucleonics sensor, and/or an RFID radiation sensor.

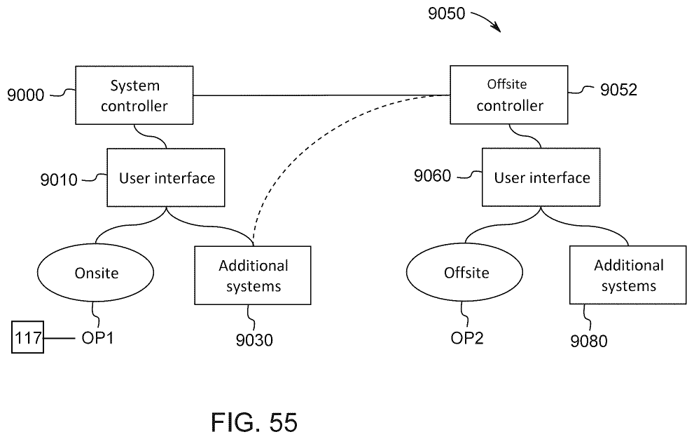

Referring now to FIG. 55, FIG. 55 illustrates a remote cockpit arrangement that provides remote access to and/or by an offsite or remote operator OP2 who may, for example be experienced and/or licensed personnel such as a nuclear medicine technologist or physician, a radiation oncologist, a health physicist, or an authorized user to enable remote personnel to oversee, control, or legally perform the preparation and delivery of the drug to meet local legal and/or regulatory requirements. Offsite may be in another room in the same facility or campus, or at some distance, including elsewhere in the same state, in the same country, or in another country. This has the benefit of enabling more sites and more operators to deliver the drug, thus expanding patient access to the drug. To enable this remote cockpit capability, an additional system 9050 is in communication with the system controller 9000. The offsite system may have an offsite controller and a user interface. The offsite controller may, for example be a laptop computer with software sufficient to enable the offsite operator to observe and/or perform all the functions that can be done through the onsite user interface 9010, preferably including checking the status of all the aspects of the drug delivery system 100 and/or fluid injector system 1000. Capabilities such as these are in common use in the IT services of many organizations as it gives the remote IT specialist access full access to a local computer. One key feature that can be used to great benefit by the offsite operator is the ability to talk face to face with the onsite operator. Capabilities such as these are available through various systems including, but not limited to, Microsoft Teams®, Zoom®, and other systems. Another key feature that can be used to great benefit by the offsite operator is the ability to communicate with the patient as if they are in the room. Another key feature that can be used to great benefit by the offsite operator is the ability to control and/or use the system camera 117 to visualize what is happening onsite and in the examining room, preferably including visually observing the status of all the aspects of the drug delivery system 100 and/or fluid injector system 1000. It is desirable for the offsite operator to be able to move and zoom the camera 117 as they feel would be most useful. The camera 117 may also be head mounted or worn by onsite operator OP1 so that the offsite operator OP2 may see exactly what the onsite operator OP1 sees. At the most basic level, the remote operator OP2 may just reply to requests for advice or help from the onsite operator OP1. At the most complete level, the remote operator OP2 may be responsible for the whole operation with the onsite operator OP1 following the directions of the remote operator OP2 and performing the activities under the offsite operator's supervision. Intermediate levels of interaction and control may be used where applicable based on expertise of the onsite operator OP1 and legal regulations.

In one embodiment, remote system 9050 comprises an offsite controller 9052, a user interface 9060, and additional systems 9080 that can be controlled by a remote operator OP2 as discussed herein. Controller 9052, interface 9060, and additional system 9080 can be the same or different than those located onsite and are formed from any of the possibilities discussed above with regard to the similarly labeled onsite components of 9000, 9010, 9030, and OP1. Further embodiments of the present disclosure may make use of various remote, or virtual, cockpits disclosed and described in detail in U.S. Provisional Patent Application No. 63/336,512, filed Apr. 29, 2022, the disclosure of which is incorporated by reference herein in its entirety.

Although embodiments or aspects have been described in detail for the purpose of illustration and description, it is to be understood that such detail is solely for that purpose and that embodiments or aspects are not limited to the disclosed embodiments or aspects, but, on the contrary, are intended to cover modifications and equivalent arrangements that are within the spirit and scope of the appended claims. Various methods of operating various systems of this disclosure are described through one or more exemplary embodiments. These methods of operation are applicable to all embodiments which provide sufficient components for such operation. In some embodiments, more components or elements may be present than are required to implement a particular method. Embodiments of this disclosure may be simplified based on the functions which are to be supported or required in a particular situation. For example, it is to be understood that the present disclosure contemplates that, to the extent possible, one or more features of any embodiment or aspect can be combined with one or more features of any other embodiment or aspect. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

We claim:

1. A radiopharmaceutical drug delivery system comprising:
(i) a movable cart;
(ii) an informatics system with a computer screen configured as a user interface;
(iii) an infusion pump; and
(iv) a configurable dose transporter, wherein the configurable dose transporter comprises:
a) a separable radioactive dose transportation and containment module;
b) a shielding for use with one or more of different radionuclide types, quantities, and volumes;
c) a radioactive dose in a vessel selected from a syringe or a vial; and
d) a disposable sealed fluid cartridge connected to the syringe or the vial via a sealable fitting.

2. The delivery system according to claim 1, wherein the vessel can be selected from different standard sizes.

3. The delivery system according to claim 1, wherein the shielding system is configured to be used with a variety of sizes of the syringe being inserted or connected.

4. The delivery system according to claim 1, wherein the computer screen is a touch screen.

5. The delivery system according to claim 1, wherein the disposable sealed fluid cartridge is configured to permit withdrawal of custom volumes from the vial or syringe based on one or more of patient weight, sex, age, other physical parameters, or health history data.

6. The delivery system according to claim 1, wherein the drug delivery system is configured to support a drug specific infusion parameter.

7. The delivery system according to claim 1, wherein the separable radioactive dose transportation and containment module is shielded.

8. The delivery system according to claim 1, wherein the disposable sealed fluid cartridge comprises a port for eliminating errors in connecting a patient line or a saline line to the cartridge.

9. The delivery system according to claim 1, wherein the system is configured to monitor and track one or more of the total volume in syringes, total volume administered in real-time, total volume from an IV bag, and total volume infused into a patient.

10. The delivery system according to claim 1, wherein the system further comprises a control system to control the process of infusion of a medical fluid into a patient.

11. The delivery system according to claim 10, wherein the control system is configured to halt the infusion process due to system error.

12. The delivery system according to claim 1, wherein the system has a feature to alert the user to occurrence of one or more of fluid occlusion, a pump failure, a deviation from a programmed volume, and an expected sequence of events in real-time.

13. The delivery system according to claim 1, wherein the shielded infusion pump is configured to protect the patient from an air infusion and comprises an alarm signal for air detection in real-time.

14. A radiopharmaceutical drug delivery system comprising:
(i) a movable cart;
(ii) an informatics system with a computer screen as a graphical user interface (GUI);
(iii) an infusion pump;
(iv) a status light system that illuminates the working area for monitoring the status of an infusion component; and
(v) a configurable dose transporter
wherein the configurable dose transporter comprises:
a) a separable radioactive dose transportation and containment module;
b) a shielding for use with one or more of different radionuclide types, quantities, and volumes;
c) a radioactive dose within a vessel selected from a syringe or a vial; and
d) a disposable sealed fluid cartridge connected to the syringe or the vial via a sealable fitting,
wherein the disposable sealed fluid cartridge is configured for withdrawing customizable volumes from the vessel.

15. The delivery system according to claim 14, wherein the status light system indicates the different state of the device with various colors.

16. The delivery system according to claim 14, wherein the status light system is controlled by a control system.

17. A radiopharmaceutical drug delivery system comprising:
a configurable dose transporter, a separable radioactive dose transportation and containment module; a shielding; a disposable sealed fluid cartridge; and a syringe or a vial;
wherein the disposable sealed fluid cartridge comprises:
(i) one or more selectable integrated flow channels;
(ii) an adapter to access the syringe or the vial;
(iii) a patient port and a port; and
(iv) one or more pressure sensors
wherein the disposable sealed fluid cartridge is configured for withdrawing a customizable volume from the syringe or the vial.

18. The delivery system according to claim 17, wherein the adapter is tagged with one or more of an RFID, barcode, or QR code.

19. The delivery system according to claim 17, wherein the disposable sealed fluid cartridge is configured for withdrawing a customizable volume based on one or more of patient weight, sex, age, health history data, and/or other physical parameter.

20. The delivery system according to claim 17, wherein the disposable sealed fluid cartridge comprises preconnected ports to eliminate errors in selection of the patient and the saline lines.
